(19) United States
(12) Patent Application Publication
(10) Pub. No.: US 2007/0122652 A1
(43) Pub. Date: May 31, 2007

(54) Compound and organic electroluminescent device using same

(75) Inventors: Masashi Hashimoto, Tokyo (JP); Shinjiro Okada, Kanagawa-ken (JP); Takao Takiguchi, Tokyo (JP); Jun Kamatani, Tokyo (JP); Satoshi Iqawa, Kanagawa-ken (JP); Minako Nakasu, Tokyo (JP); Hironobu Iwawaki, Kanagawa-ken (JP); Ryota Ooishi, Kanagawa-ken (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Ohta-ku, Tokyo (JP)

(21) Appl. No.: 10/583,770

(22) PCT Filed: Sep. 28, 2005
(86) PCT No.: PCT/JP05/18393

§ 371 Date: Jun. 21, 2006

(30) Foreign Application Priority Data
Sep. 29, 2004 (JP) 2004-283238
Aug. 12, 2005 (JP) 2005-234360

(51) Int. Cl. H01L 51/54; C07C 13/567; C09K 11/06

(52) U.S. Cl. 428/690; 428/917; 313/504; 313/506; 257/102; 257/103; 257/E51.049; 585/27

(57) ABSTRACT

Provided is a novel compound that can be suitably used as a compound for an organic EL device. The compound is represented by general formula (1): 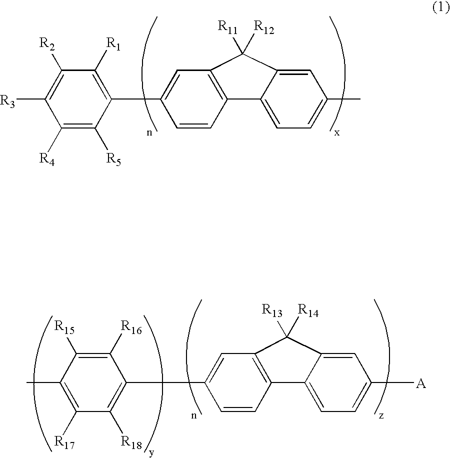 wherein x, y and z are an integer of 0 to 3 with x+z>1;

R₃, R₁₅, R₁₆, R₁₇, and R₁₈ are hydrogen or a linear or branched alkyl;

R₁, R₂, R₄, and R₅ are hydrogen, a linear or branched alkyl, or a substituted or unsubstituted aryl with at least one being a substituted or unsubstituted aryl;
A is hydrogen, a linear or branched alkyl, or group B: 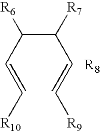
(wherein R₆, R₇, R₈, R₉, and R₁₀ are hydrogen, a linear or branched alkyl, or a substituted or unsubstituted aryl);

R₁₁, R₁₂, R₁₃, and R₁₄ are hydrogen, a linear or branched alkyl, or a substituted or unsubstituted aryl; and each CH on the benzene ring may be replaced by nitrogen.

### TECHNICAL FIELD

The present invention relates to a light-emitting device using an organic compound, and more particularly to a novel compound having a specific molecular structure and an organic electroluminescent (EL) device using the same.

### BACKGROUND ART

In an old example of an organic light-emitting device, a voltage is applied to an anthracene evaporated film to emit light (Thin Solid Films, 94 (1982), 171). In addition, applied research on an organic light-emitting device has been vigorously conducted.

As detailed in Macromol. Symp. 125, 1 to 48 (1997), an organic EL device is generally, structured to have two (upper and lower) electrodes formed on a transparent substrate and an organic substance layer including a light-emitting layer formed between the electrodes.

In addition, investigation has been recently made into a device using not only conventional light emission utilizing fluorescence upon transition from singlet exciton to ground state but also phosphorescence via triplet exciton as typified by D. F. O'Brien et al, “Improved energy transfer in electrophosphorescent device”, Applied Physics Letters, Vol. 74, No. 3, p. 442 (1999) and M. A. Baldo et-al, “Very high-efficiency green organic light-emitting devices based on ectrophosphorescence”, Applied Physics Letters, Vol. 75, No. 1, p. 4 (1999). In each of these documents, an organic layer having a four-layer structure is mainly used. The structure is composed of a hole-transporting layer, a light-emitting layer, an exciton diffusion-prevention layer, and an electron-transporting layer stacked in the mentioned order from an anode side. The materials used are carrier transporting materials and a phosphorescence emitting material Ir(ppy)₃ shown below.

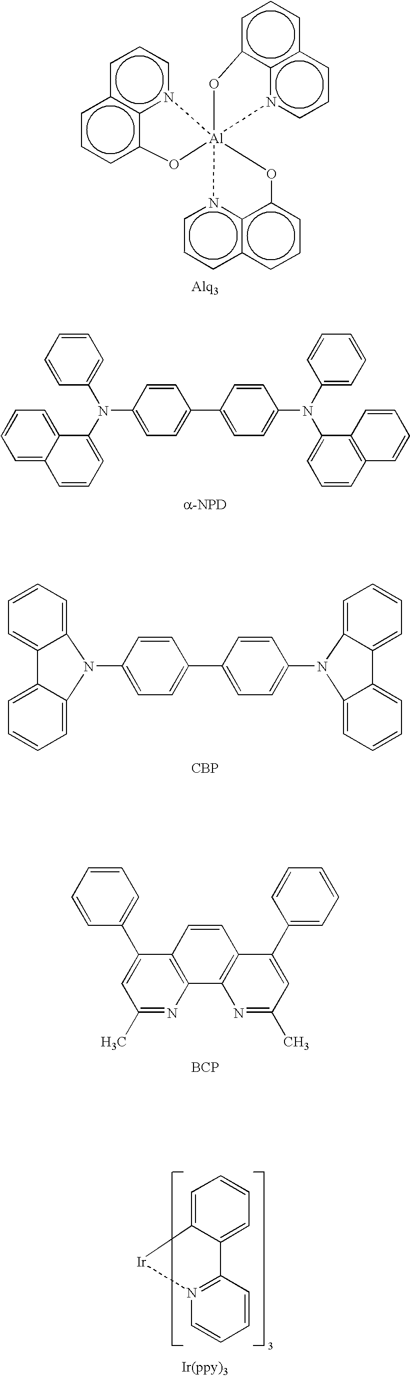

Further, emission of a light from ultraviolet to infrared region can be performed by changing the kind of a fluorescent organic compound. In these days, research has been actively made on various compounds.

In addition to organic light-emitting devices using such low-molecular materials as those described above, a group of the University of Cambridge has reported organic light-emitting devices using conjugate polymers (Nature, 347, 539 (1990)). This report has confirmed that light emission can be obtained by a single layer by forming polyphenylene vinylene (PPV) in a film shape by use of an application system.

As described above, recent progress of an organic light-emitting device is remarkable, and is characterized in that a highly responsive, thin, and lightweight light-emitting device that can be driven at a low applied voltage and provides a high luminance and a variety of emission wavelengths can be made, which suggests the applicability to a wide variety of uses.

However, at present, an optical output of a higher luminance or a higher conversion efficiency has been required. In addition, there still remain a large number of problems in terms of durability such as a change over time due to long-term use and deterioration due to an atmospheric gas containing oxygen or to moisture. Furthermore, light emission of blue, green and red colors having a high color purity is necessary when application to a full-color display or the like is attempted. However, those problems have not been sufficiently solved yet.

In addition, a large number of aromatic compounds and condensed polycyclic aromatic compounds have been studied as fluorescent organic compounds used for an electron-transporting layer, a light-emitting layer, and the like. However, it is difficult to say that a compound sufficiently satisfying emission luminance and durability has been already obtained.

Examples of patent documents describing application of a fluorene compound to an organic EL, which is related to the present invention, include JP 2004-43349A, WO 99/54385, and JP 2003-229273A. However, none of the patent documents discloses an organic compound of the present invention characterized by including a partial structure containing a fluorene ring and a phenylene ring on a straight line in a molecular structure. In addition, a fluorene compound has been reported as application to a laser dye (Journal of Fluorescence, Vol. 5, No. 3, 295 (1995)).

In order to apply an organic EL device to a display unit of a display apparatus or the like, the device is required to have an optical output of a high efficiency and a high luminance and sufficiently secure high durability. However, such requirement has not been sufficiently met.

### DISCLOSURE OF THE INVENTION

It is, therefore, an object of the present invention to provide a novel compound that can be suitably used as a compound for an organic EL device.

Another object of the present invention is to provide an organic EL device using the compound and having an optical output of a high efficiency and a high luminance.

Still another object of the present invention is to provide an organic EL device with high durability.

Yet another object of the present invention is to provide an organic EL device that can be produced easily at a relatively low cost.

That is, according to one aspect of the present invention, there is provided a compound represented by the general formula (1): 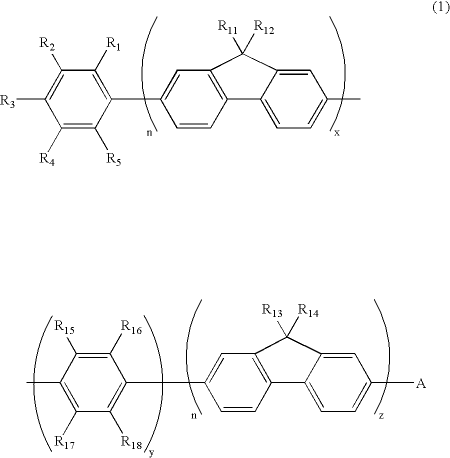
wherein

x, y and z are each independently an integer of 0 to 3 with the proviso that the relation of x+z≧1 is satisfied;

R₃, R₁₅, R₁₆, R₁₇, and R₁₈ are each independently a hydrogen atom or a linear or branched alkyl group, and each CH on the benzene ring having R₁₅, R₁₆, R₁₇, and R₁₈ may independently be replaced by a nitrogen atom;

R₁, R₂, R₄, and R₅ are each independently a hydrogen atom, a linear or branched alkyl group, or a substituted or unsubstituted aryl group with the proviso that at least one of R₁, R₂, R₄, and R₅ is a substituted or unsubstituted aryl group, and each CH on the benzene skeleton constituting the aryl group and each CH on the benzene ring having R₁, R₂, R₃, R₄, and R₅ may independently be replaced by a nitrogen atom;

A is a hydrogen atom, a linear or branched alkyl group, or group B represented by the general formula: 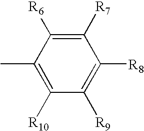
(wherein R₆, R₇, R₈, R₉, and R₁₀ are each independently a hydrogen atom, a linear or branched alkyl group, or a substituted or unsubstituted aryl group, and each CH on the benzene ring having R₆, R₇, R₈, R₉, and R₁₀ and each CH on the benzene skeleton constituting the aryl group may independently be replaced by a nitrogen atom); and

R₁₁R₁₂, R₁₃, and R₁₄ are each independently a hydrogen atom, a linear or branched alkyl group, or a substituted or unsubstituted aryl group.

According to another aspect of the present invention, there is provided an organic electroluminescent device comprising a pair of electrodes, and at least one layer comprising an organic compound provided between the pair of electrodes, wherein at least one of the at least one layer comprising the organic compound comprises at least one of the compounds represented by the general formula (1).

The compound of the present invention has a high glass transition temperature. In addition, when the skeleton composed of the phenyl rings and the fluorene rings is defined as a major axis of the molecule (hereinafter, referred to as “molecular major axis”), by lowering the crystallinity by means of aryl substituents extending in a sideward direction from the molecular major axis, the stabilization as in an amorphous film structure can be expected.

The compound of the present invention is expected to be advantageous in terms of conductivity over one having crystallinity reduced by adding linear or branched long-chain alkyl groups. Furthermore, the compound is expected to have a higher solubility in an organic solvent than that of a compound of a straight molecular structure having no aryl substituent extending in a sideward direction from the molecular major axis, so that various purification methods are expected to be applicable thereto.

The light-emitting device of the present invention using the compound of the present invention for a host of a light-emitting layer is an excellent device capable of emitting light with a high efficiency and maintaining a high luminance for a longer time period than that of a compound conventionally used. In addition, the light-emitting device shows an increased current value at the same voltage value as compared to a conventional device, so it is expected to be driven at a lower voltage.

### BRIEF DESCRIPTION OF THE DRAWINGS

FIGS. 1A, 1B and 1C are schematic views showing an example of the light-emitting device in accordance with the present invention.

### BEST MODE FOR CARRYING OUT THE INVENTION

First, the compound of the present invention will be described.

When a light-emitting layer comprises a carrier transporting host material and a guest, the process for light emission is composed of the following several steps.

1. Transportation of electrons/holes in the light-emitting layer

2. Generation of excitons in the host

3. Transmission of excitation energy between host molecules

4. Transfer of the excitation energy from the host to the guest

The desired energy transfer and light emission in the respective steps are caused in competition with various deactivation steps.

It is needless to say that in order to increase the emission efficiency of an EL device, the emission quantum yield of a luminescent center material itself must be large. However, how high efficiency of energy transfer between hosts or between a host and a guest can be achieved is also a large problem. In addition, the cause for deterioration of light emission due to energization has not been clarified yet. However, it is assumed that the deterioration is related at least to a luminescent center material itself or an environmental change of a light-emitting material due to surrounding molecules.

In view of the above, the inventors of the present invention have made various studies to find that a device using the compound represented by the general formula (1) as a host of a light-emitting layer emits light with a high efficiency, maintains a high luminance for a long period of time, and shows less deterioration due to energization.

One possible cause for the deterioration of light emission due to energization is deterioration of light emission due to deterioration of a thin-film shape of a light-emitting layer. It is believed that the deterioration of the thin-film shape results from crystallization of an organic thin film due to a temperature of drive environment or heat generation at the time of driving a device. This is considered to originate from a low glass transition temperature of a material and a high crystallinity of a host compound, so that an organic EL material is required to have a high glass transition temperature and high stability of an amorphous film state.

The compound of the present invention has a high glass transition temperature and its crystallinity is reduced by an aryl substituent extending in a sideward direction from the molecular major axis. As a result, the amorphous film state is stabilized, so that the durability of an organic EL device is expected to increase.

The term “major axis” herein employed refers to an axis parallel to the direction in which a benzene ring and a fluorene skeleton constituting a main skeleton in the general formula (1) are bonded to each other in the main skeleton structure.

More specifically, the major axis is defined as the direction that connects the position having none of R₁ to R₅ bonded of positions 1 to 6 of the benzene ring having R₁ to R₅ and position 2 or 7 of the fluorene skeleton which is adjacent and bonded to the benzene ring.

The fluorene skeleton is bonded at position 2 or 7 thereof to another skeleton. An axis parallel to the binding direction (direction connecting positions 2 and 7) is defined as the major axis.

Further, in the benzene ring having R₁₅ to R₁₈, the direction connecting two positions each having none of R₁₅ to R₁₈ bonded (two positions that can be represented as positions 1 and 4 of the benzene ring when the position at which the benzene ring is bonded to the foregoing fluorene skeleton assumed to be position 1) is defined as the major axis.

Moreover, an axis parallel to the direction connecting positions 2 and 7 of the fluorene skeleton bonded to that benzene ring and to A in the general formula 1 is defined as the major axis.

In addition, when A in the general formula (1) is the group B, the major axis is defined as the direction that connects the position having none of R₆ to R₁₀ bonded of positions 1 to 6 of the benzene ring having R₆ to R₁₀ and position 2 or 7 of the fluorene skeleton which is adjacent and bonded to the benzene ring.

The term “sideward” herein employed refers to, in the case of the benzene ring having R₁ to R₅, the direction in which at least one of R₁, R₂, R₄, and R₅ is bonded to the benzene ring.

Alternatively, the term “sideward” refers to, in the case of the benzene ring having R₁₅ to R₁₈, the direction in which at least one of R₁₅, R₁₆, R₁₇, and R₁₈ is bonded to the benzene ring.

Alternatively, the term “sideward” refers to, in the case of the benzene ring having R₆ to R₁₀ of group B, the direction in which at least one of R₆, R₇, R₉, and R₁₀ is bonded to the benzene ring.

The compound in accordance with the present invention is represented by the general formula (1). In particular, a compound in which A is a hydrogen atom or group B, specifically a compound represented by the following general formula (2) or (3) is preferable. In addition, a compound in which both y and z are 0, specifically a compound represented by the following general formula (4) or (5) is more preferable.

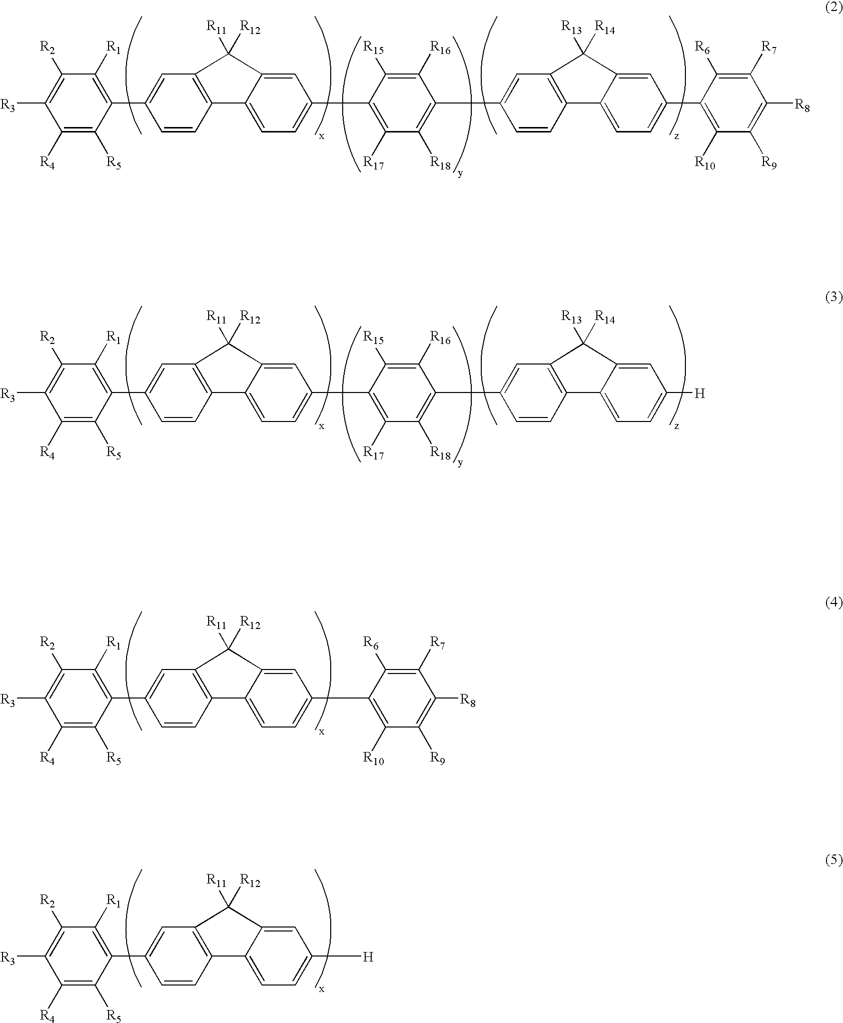

In the general formula (1), it is preferred that the substituents (R₁₁, R₁₂, R₁₃, and R₁₄) bonded to the position 9 of any fluorene group (fluorene skeleton) are each independently a hydrogen atom, a linear or branched alkyl group, or a substituted or unsubstituted aryl group.

The substituents are more preferably a linear or branched alkyl group, still more preferably methyl group or ethyl group, and still further more preferably methyl group. In particular, when the substituents each bonded to position 9 of the fluorene group, that is, R₁₁ to R₁₄ each represent methyl group, a higher glass transition temperature and high heat resistance are can be attained, so that the durability of an organic EL device is expected to increase. Further, in order to obtain a device capable of emitting light with a high efficiency, the drive voltage needs to be lowered. To this end, it is important that a host has charge conductivity. When an alkyl chain is bonded to position 9 of the fluorene group, it is considered that lengthening the alkyl chain reducing the charge conductivity. Therefore, when the substituent bonded to position 9 of the fluorene group is methyl, higher charge conductivity can be provided and the drive voltage of a device can be lowered, so that the efficiency of an organic EL device is expected to be increased.

R₁₅, R₁₆, R₁₇, and R₁₈ are each independently a hydrogen atom or a linear or branched alkyl group with a hydrogen atom or methyl group being preferred in the viewpoint of the glass transition temperature and charge conductivity as with the above.

R₁, R₂, R₄, R₅, R₆, R₇, R₈, R₉, and R₁₀ are each independently a hydrogen atom, a linear or branched alkyl group, or a substituted or unsubstituted aryl group, and at least one of R₁, R₂, R₄, and R₅ is a substituted or unsubstituted aryl group.

Each CH on the benzene skeleton constituting the aryl group may independently be replaced by a nitrogen atom.

Preferable examples of the aryl group or the substituent having CH on the benzene skeleton constituting the aryl group replaced by a nitrogen atom include phenyl group, naphthyl group, anthranil group, fluorenyl group, pyrenyl group, phenanthrenyl group, crysenyl group, fluoranthenyl group, triphenylenyl group, pyridyl group, pyrazinyl group, pyrimidyl group, pyridazinyl group, quinolinyl group, isoquinolinyl group, phenanthridinyl group, acridinyl group, naphthylidinyl group, quinoxalinyl group, quinazolinyl group, cinnolinyl group, phthaladinyl group, phenanthrolyl group, and phenadinyl group. More preferable examples thereof include phenyl group, naphthyl group, fluorenyl group, pyridyl group, pyrazinyl group, pyrimidyl group, quinolinyl group, isoquinolinyl group, quinoxalinyl group, and phenanthrolyl group. Still more preferable examples thereof include phenyl group, naphthyl group, and fluorenyl group. An aryl group may also be used which is formed by combining at least two of the aryl groups and the substituents each havlng CH on the benzene rings constituting the aryl group replaced by a nitrogen atom through formation of a bond at arbitrary positions, and a substituent having CH on the benzene skeleton constituting the aryl group replaced by a nitrogen atom is also available. Examples of the substituent for the aryl group or for the substituent having CH on the benzene skeleton constituting the aryl group replaced by a nitrogen atom preferably include a linear or branched alkyl group, more preferably include methyl group or ethyl group, and still more preferably include methyl group from the viewpoint of the charge conductivity. Incidentally, from the viewpoint of the charge conductivity, it is also preferred that the aryl group or the substituent not substituted.

Preferable examples of the alkyl group include methyl group and ethyl group, with methyl group being more preferred.

The provision of aryl substituent(s) extending in a sideward direction from the molecular major axis makes the molecular shape bulky, so that the crystallinity is expected to be lowered and the stability of an amorphous state is expected to improve. In addition, since an intermolecular action due to a π-π interaction can be expected from an aryl group, the improvement of the amorphous property can be expected while suppressing reduction in the glass transition temperature.

Another possible cause for the deterioration of light emission due to energization is contamination with an impurity. When a polymer compound is used for a device, since it is difficult to remove impurities in the polymer compound, the impurities are apt to contaminate the device, thereby shortening the lifetime of the device. Because the compound in accordance with the present invention is a single compound, appropriate use of a purification method such as recrystallization, column chromatography, or sublimation purification can facilitate the removal of impurities and is expected to improve the durability of an organic EL device.

Specific structural formulae of the compound in accordance with the present invention are shown below. However, they are merely representative examples and the present invention is not limited thereto.
<Exemplified Compound No. X-1 to X-394>

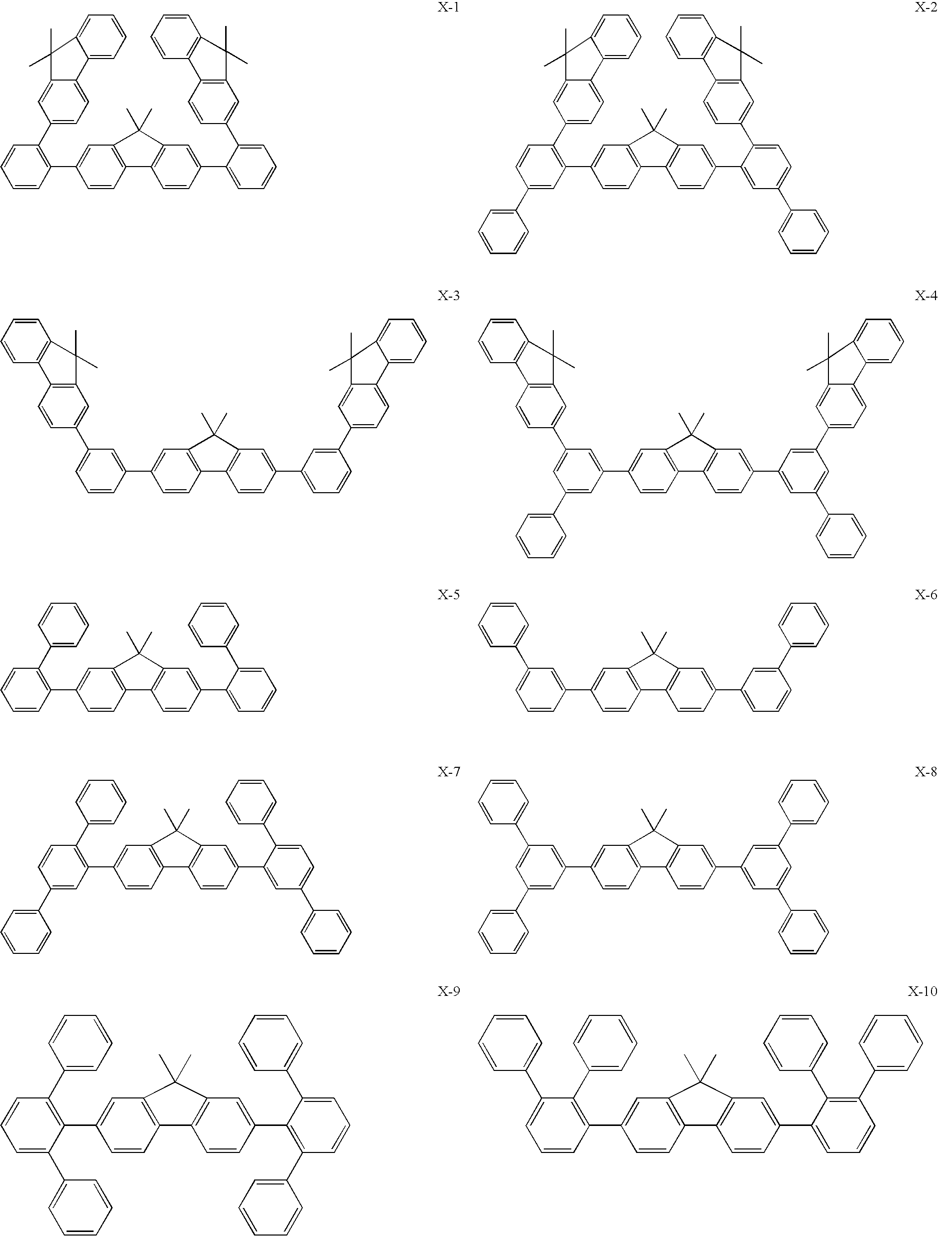

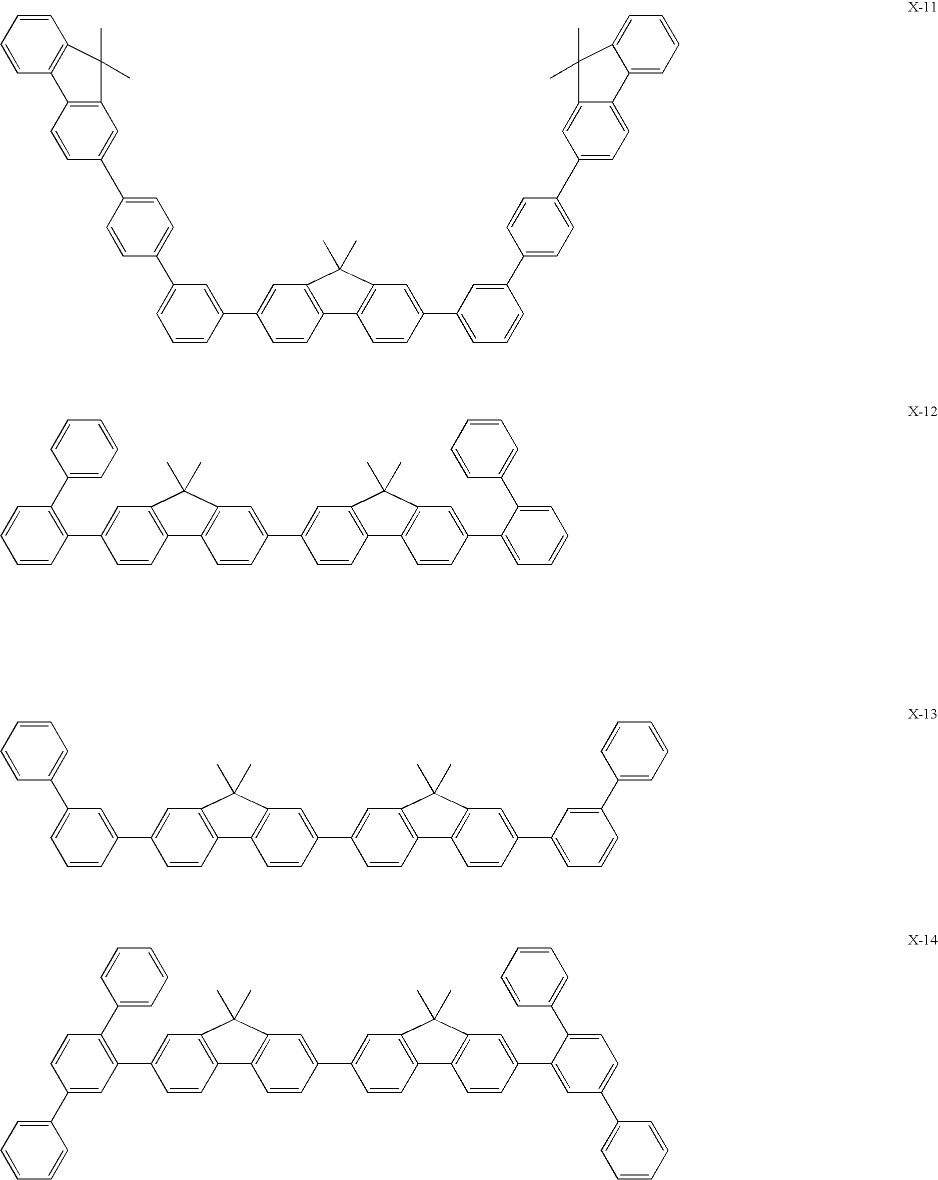

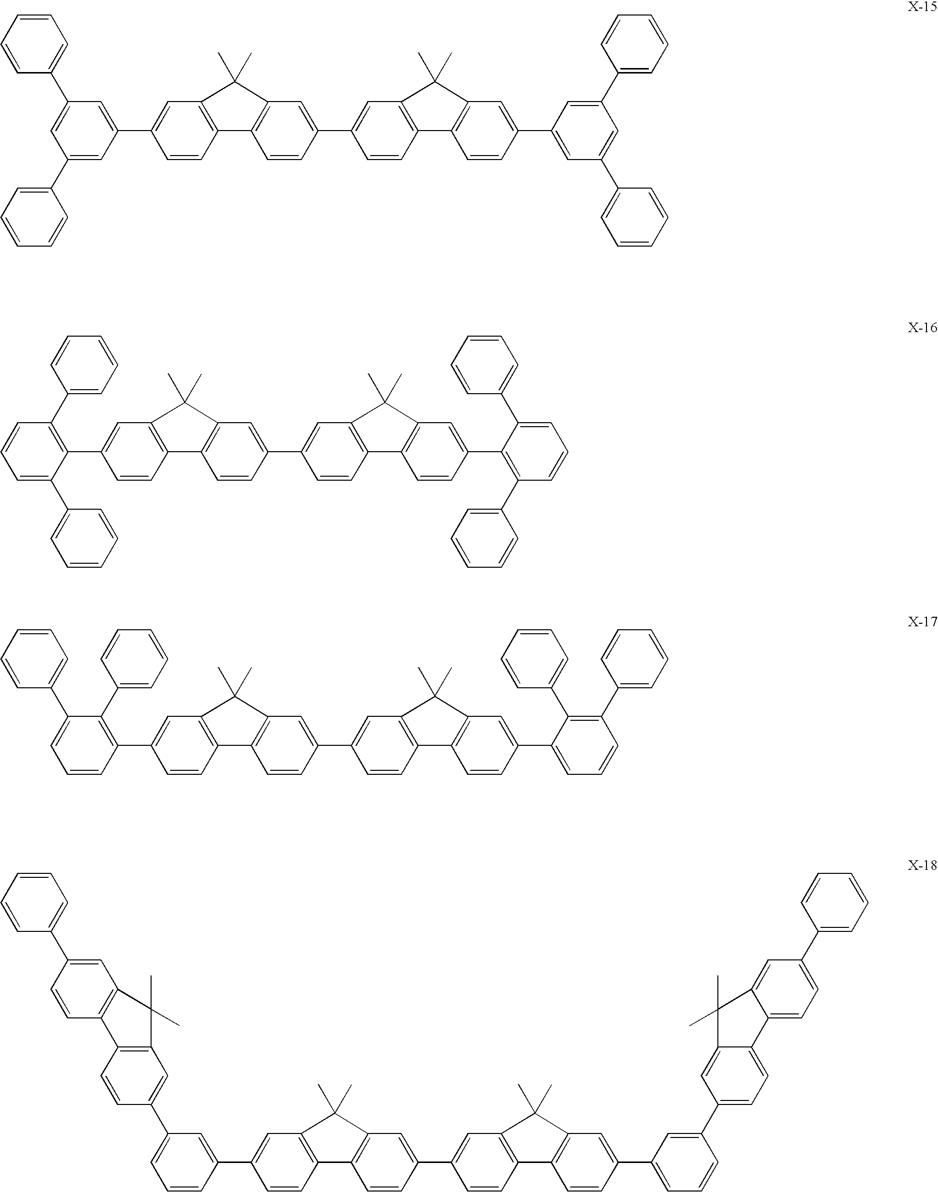

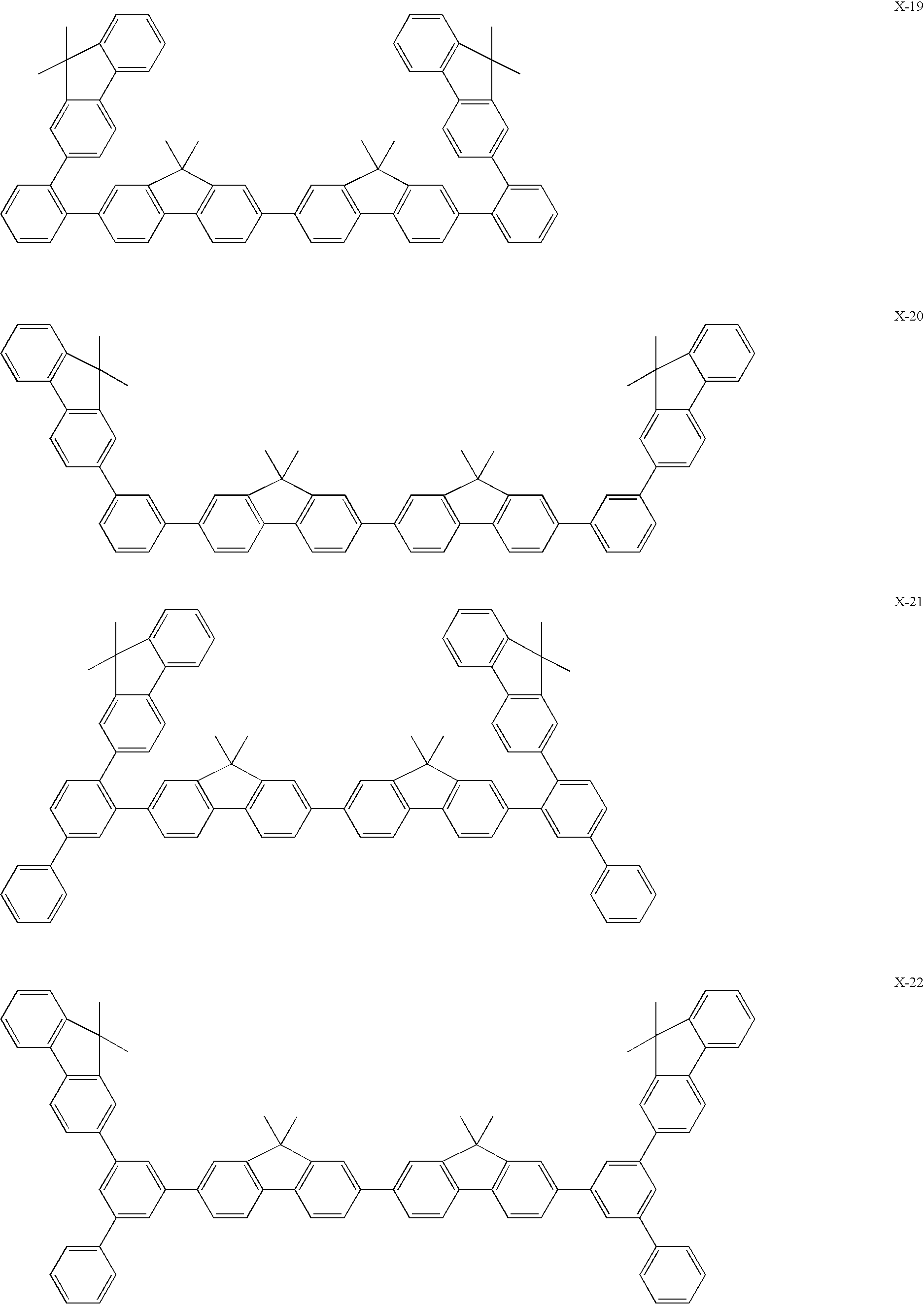

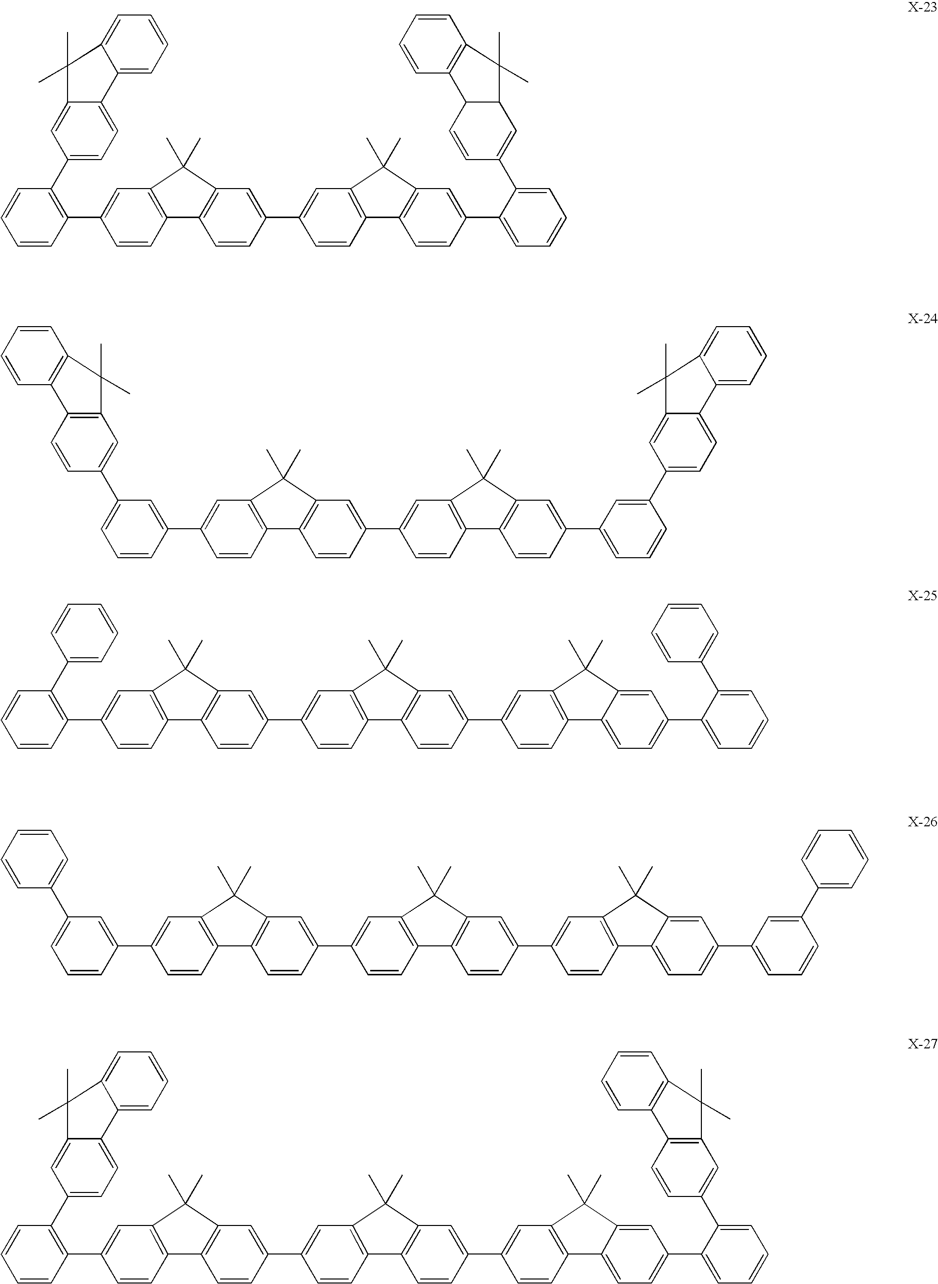

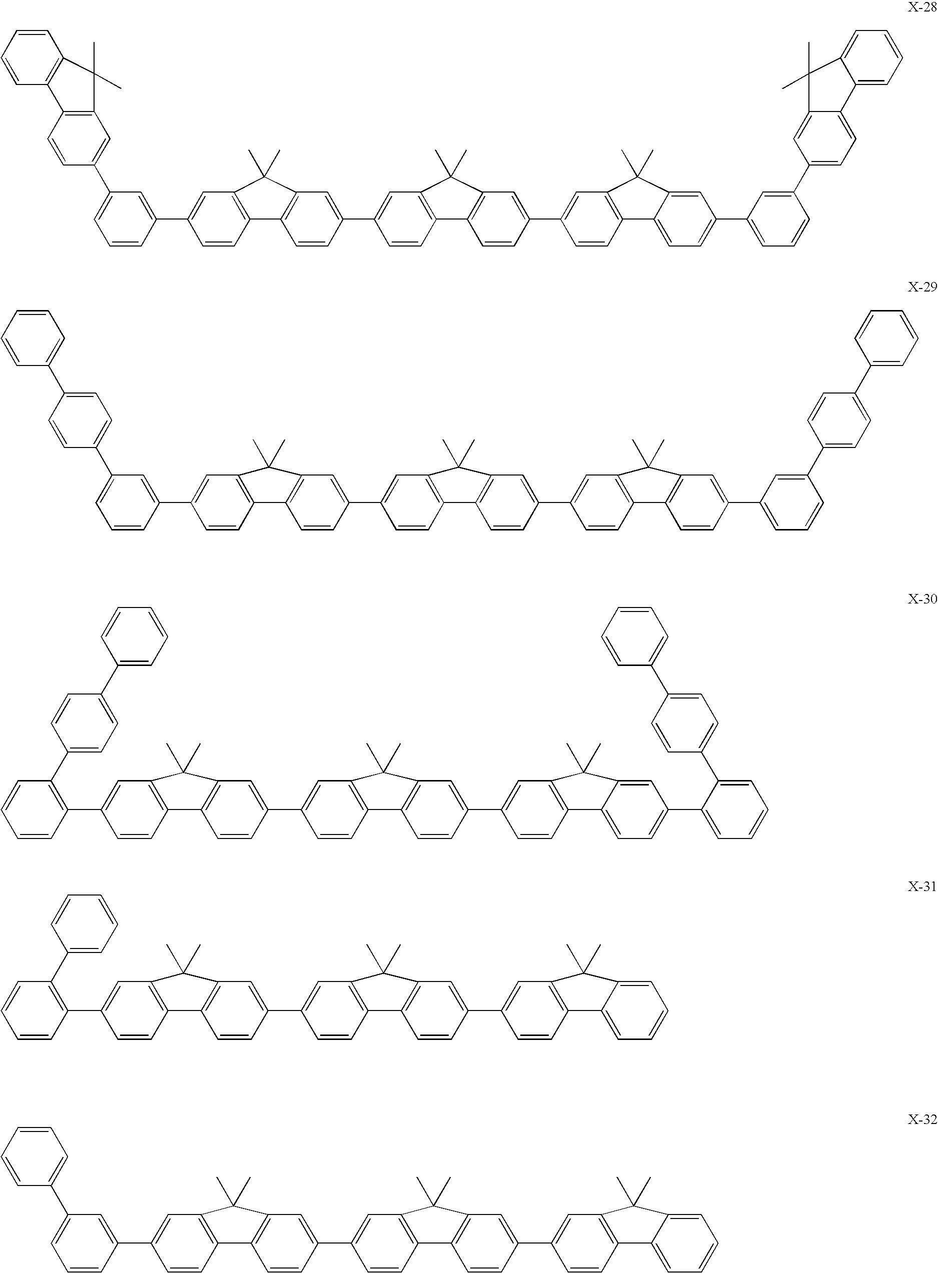

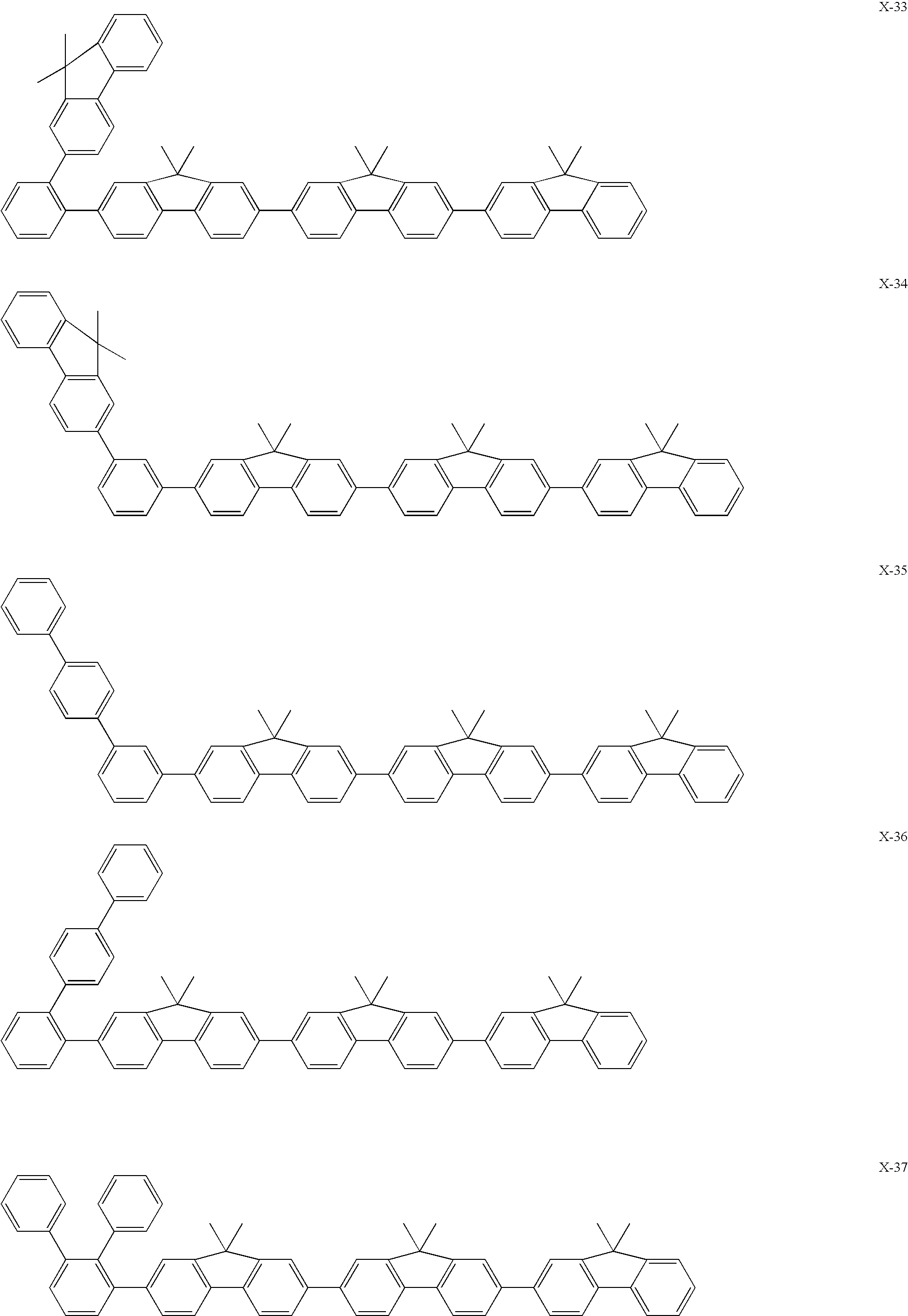

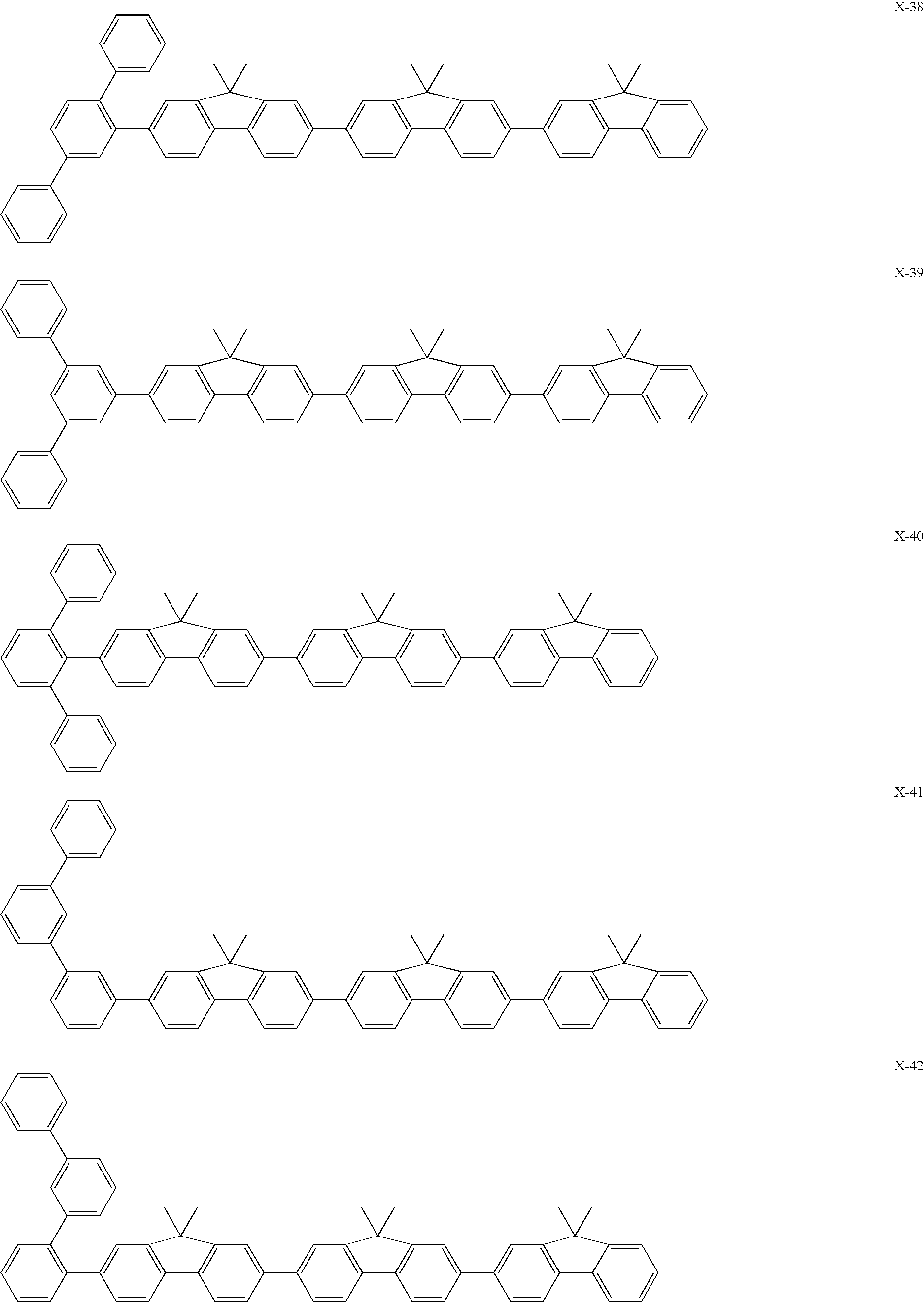

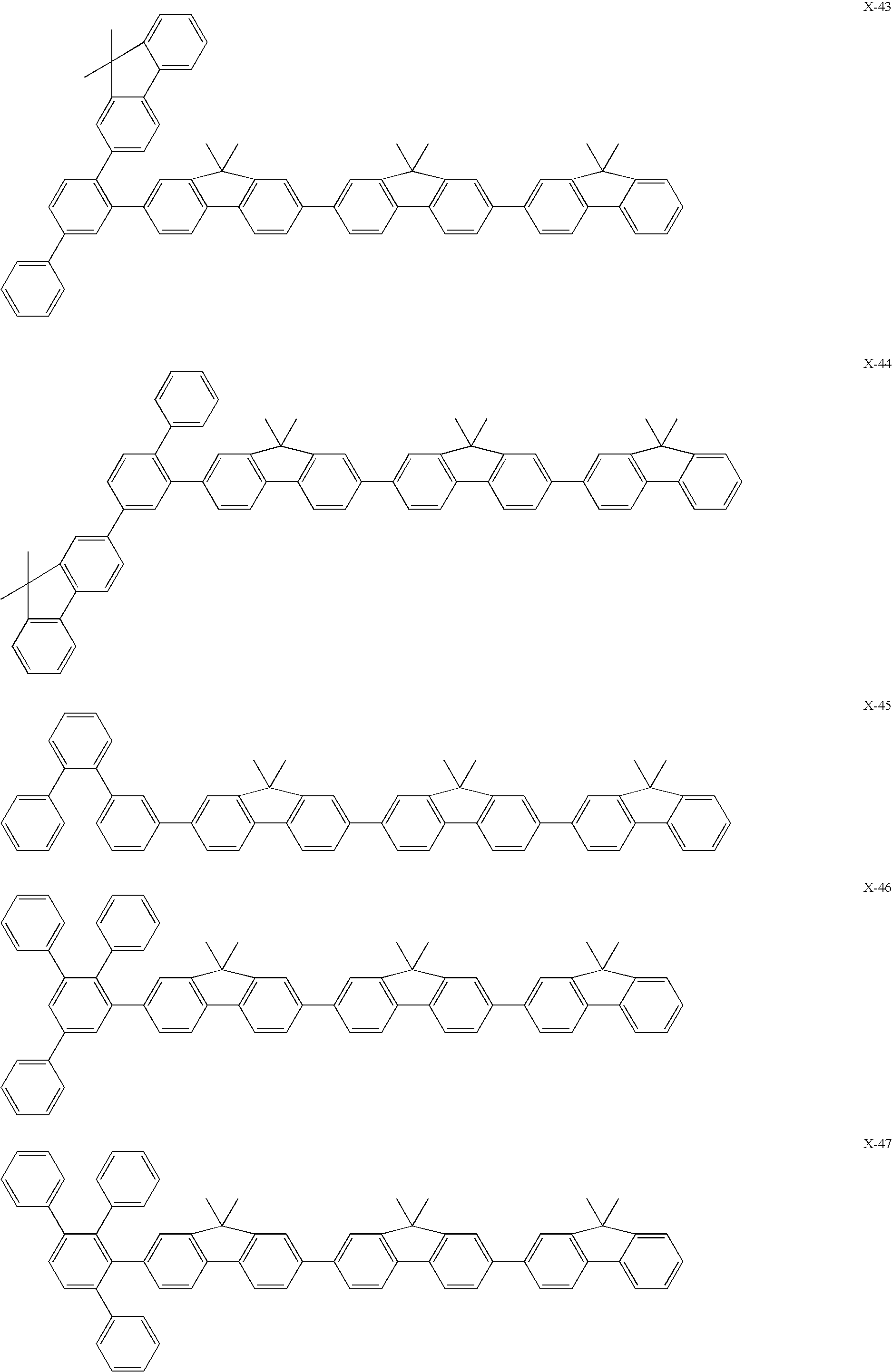

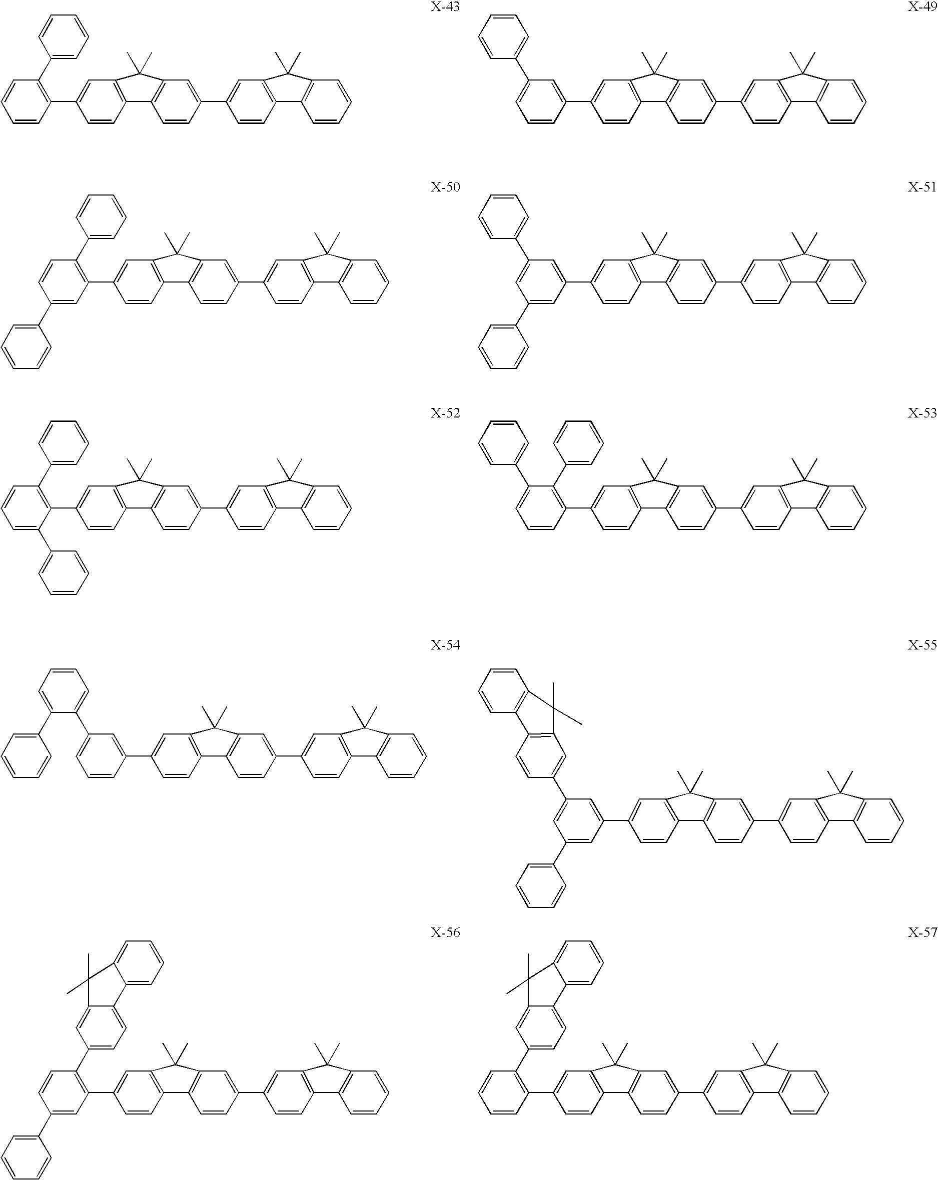

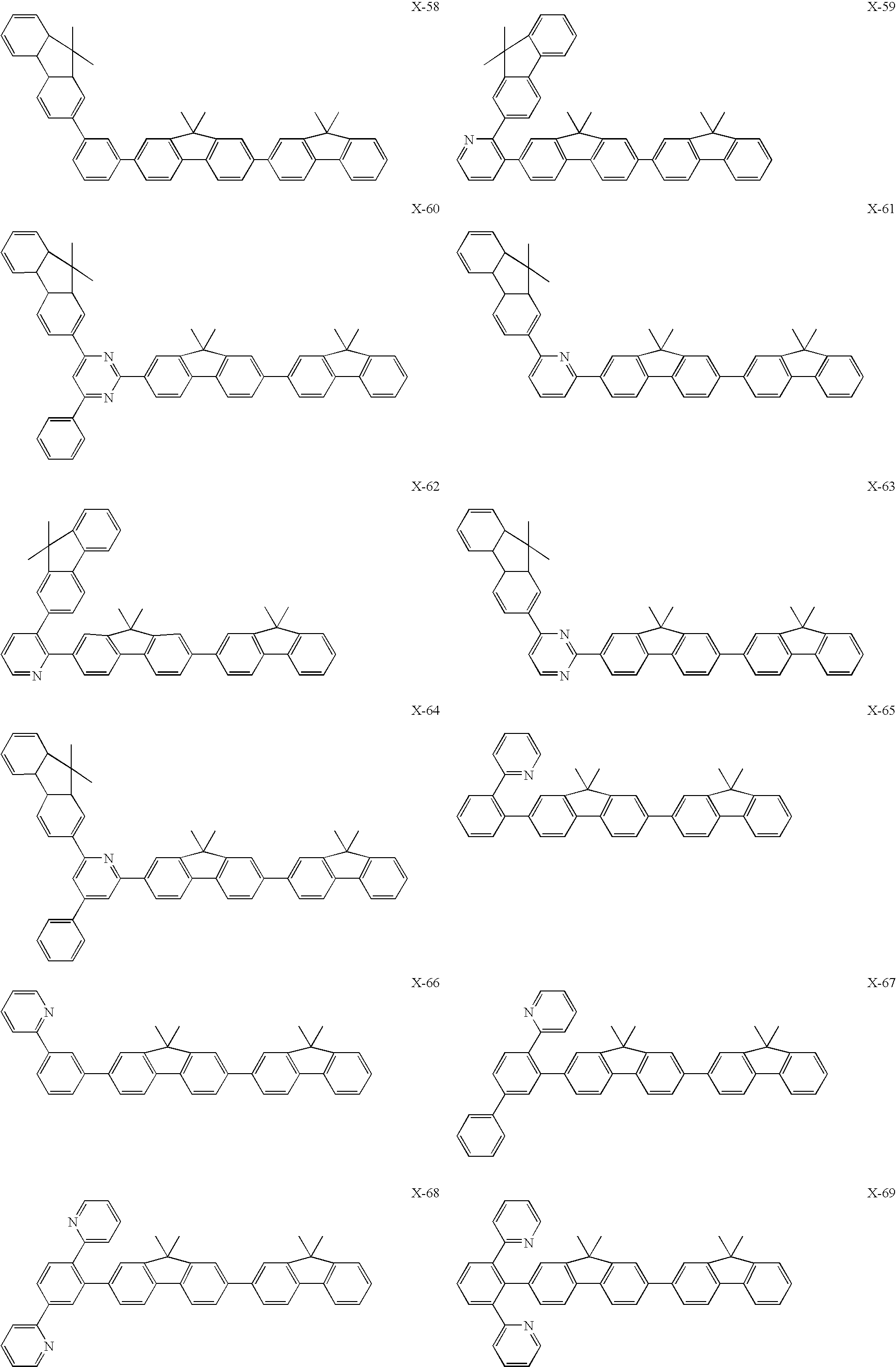

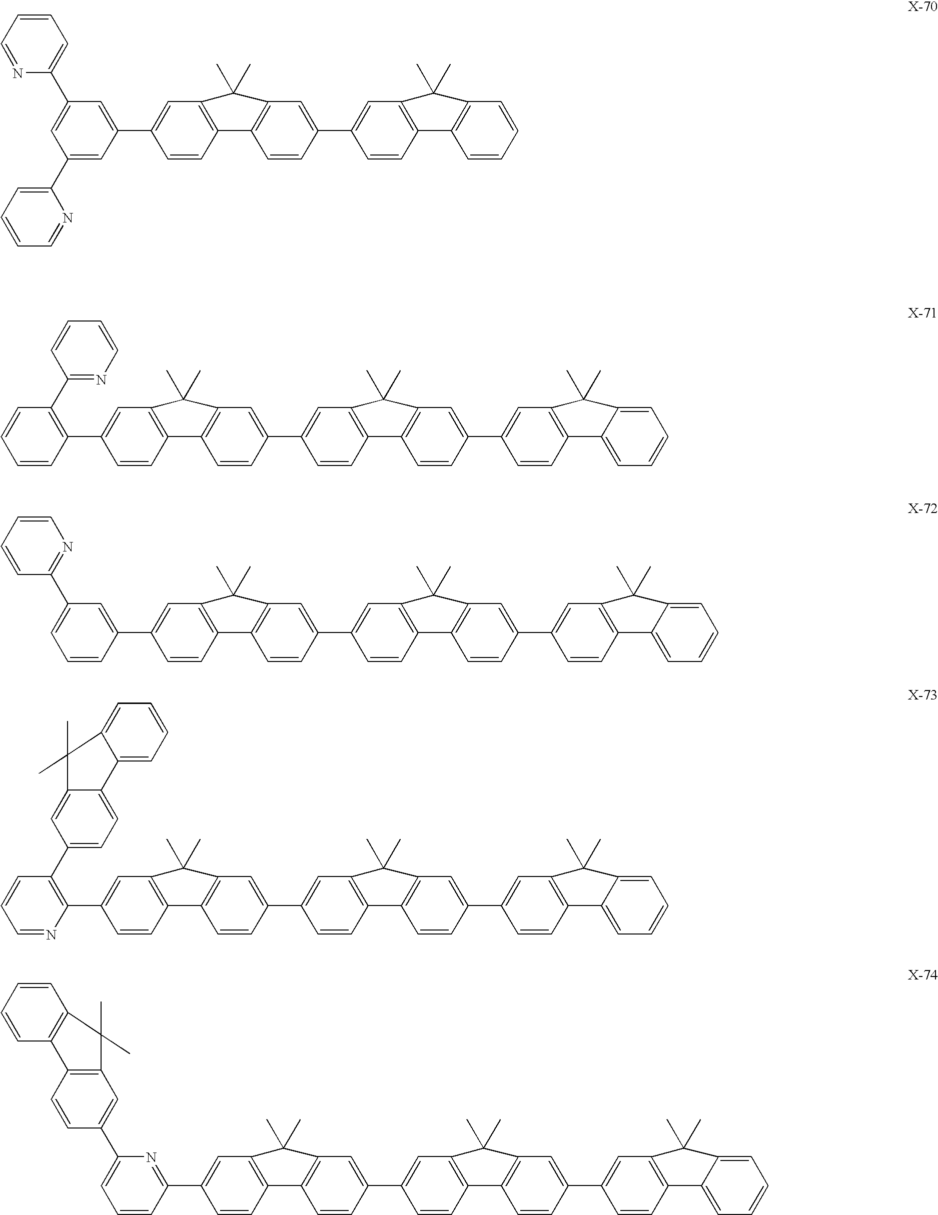

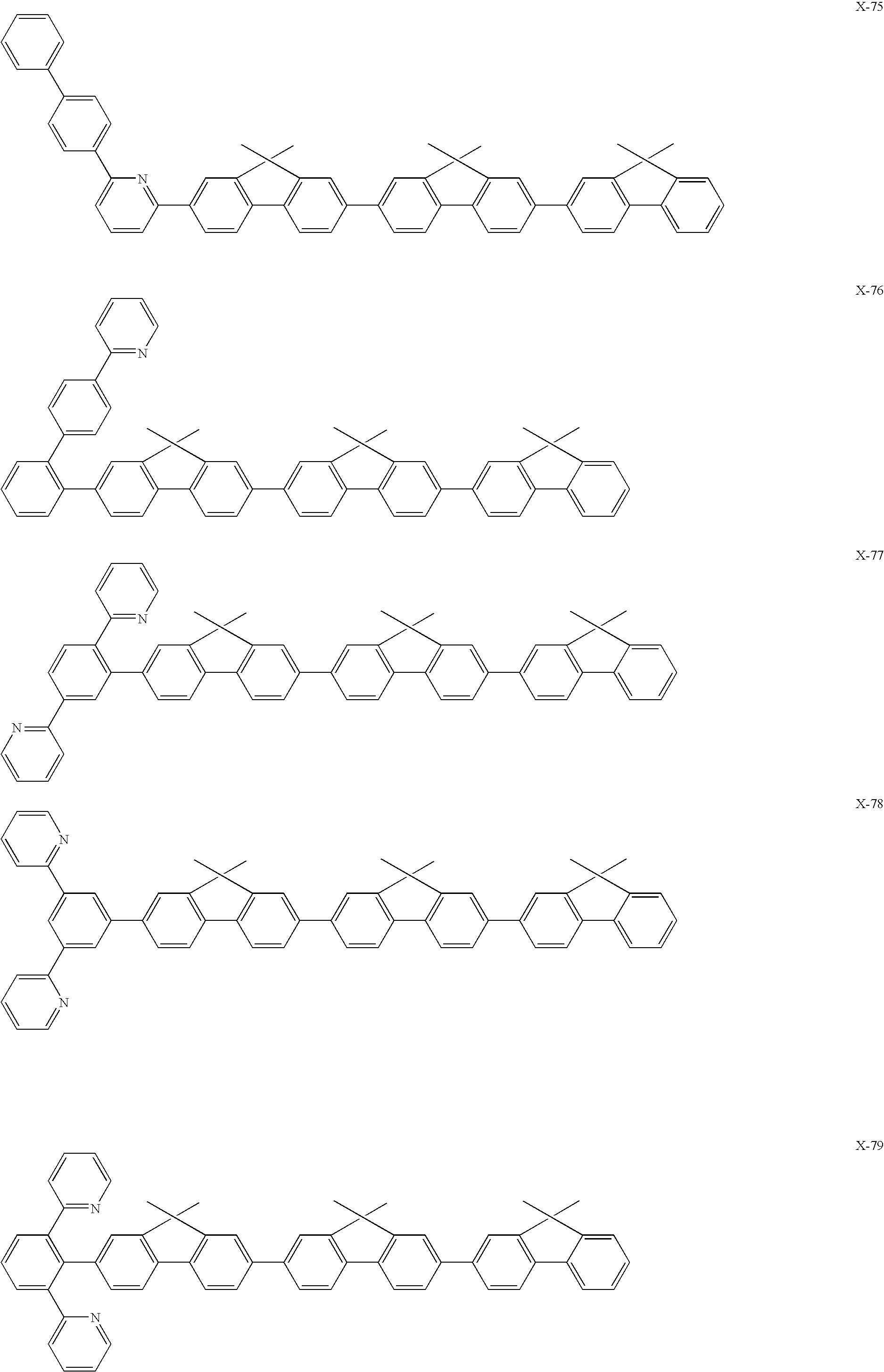

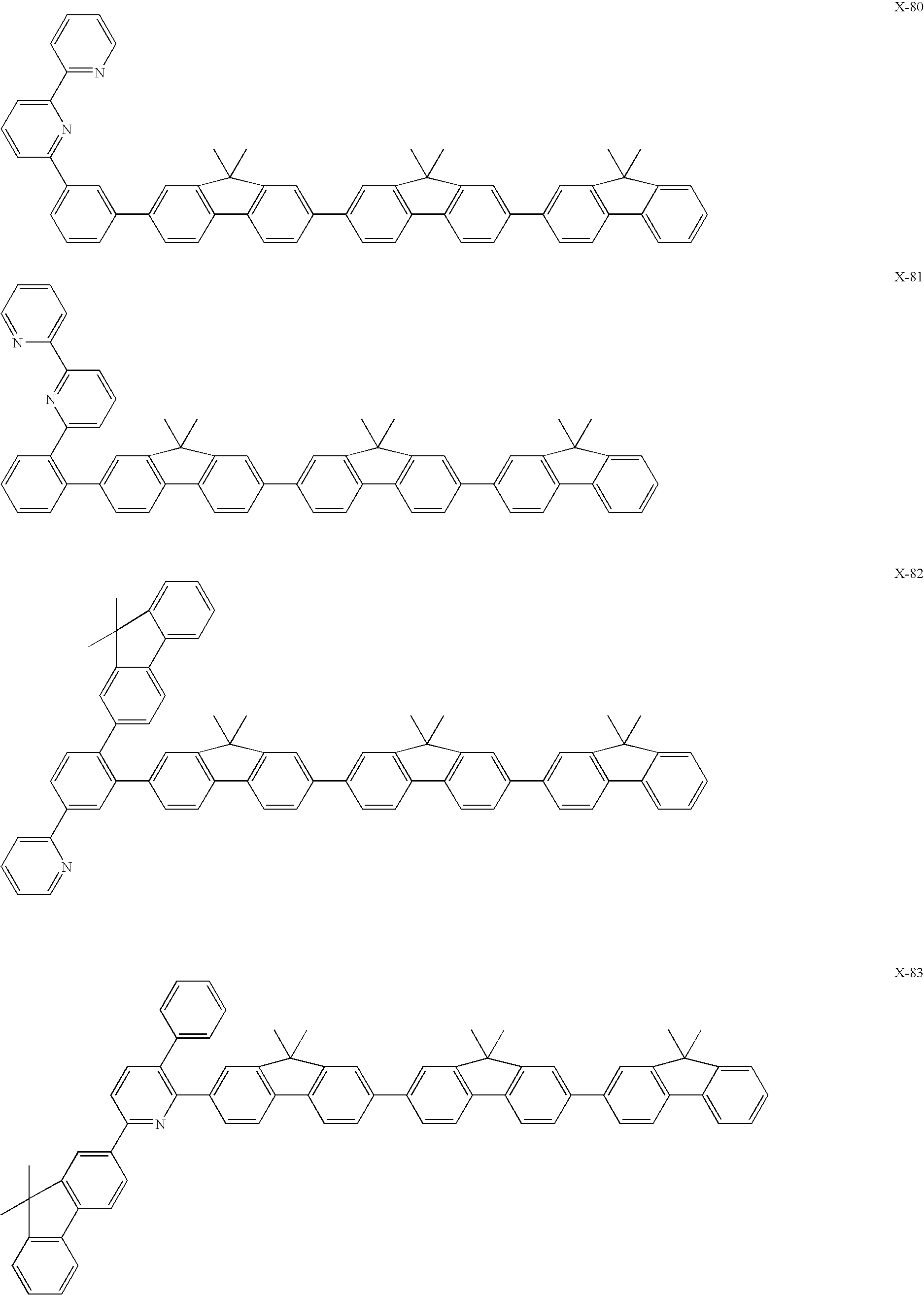

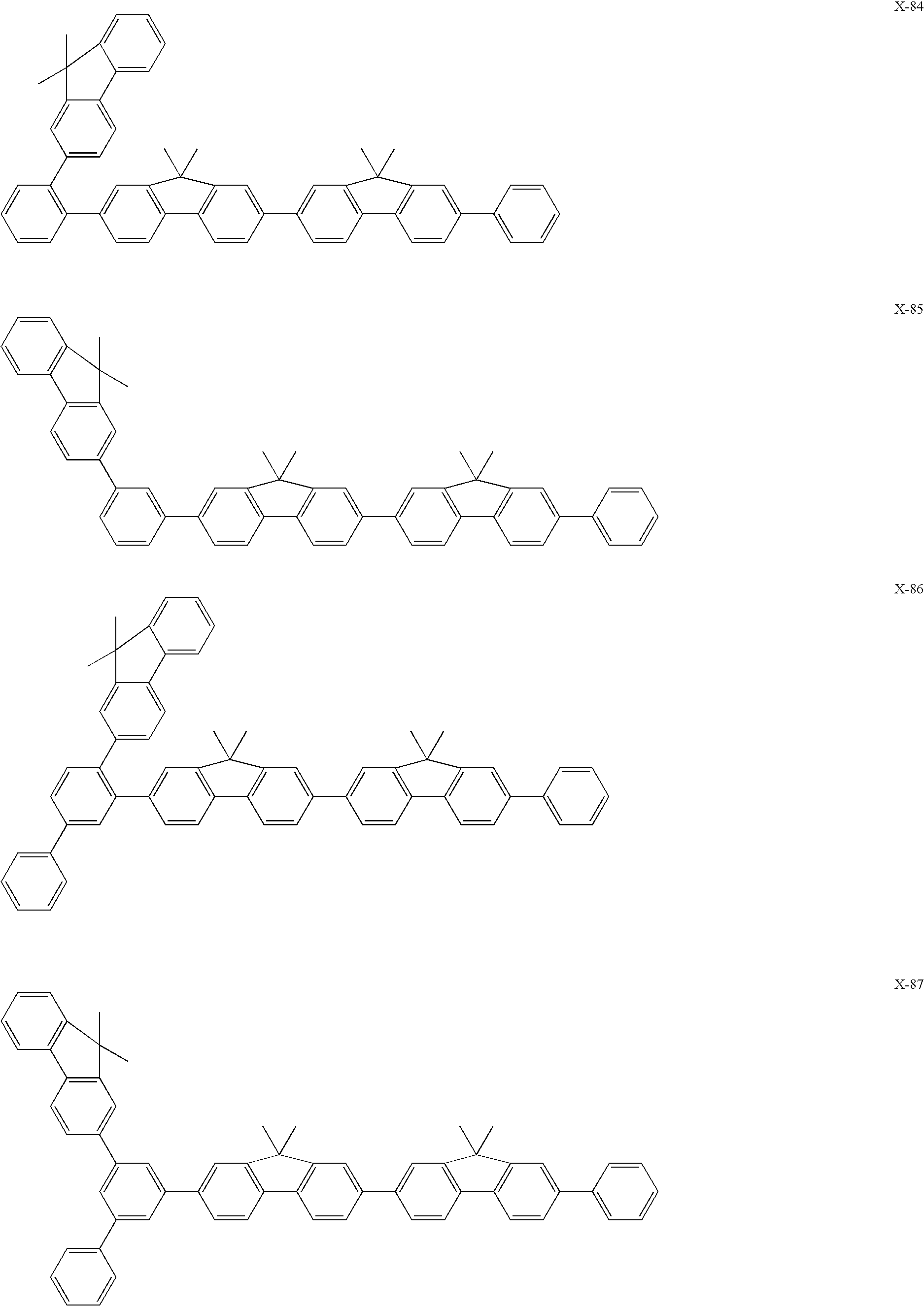

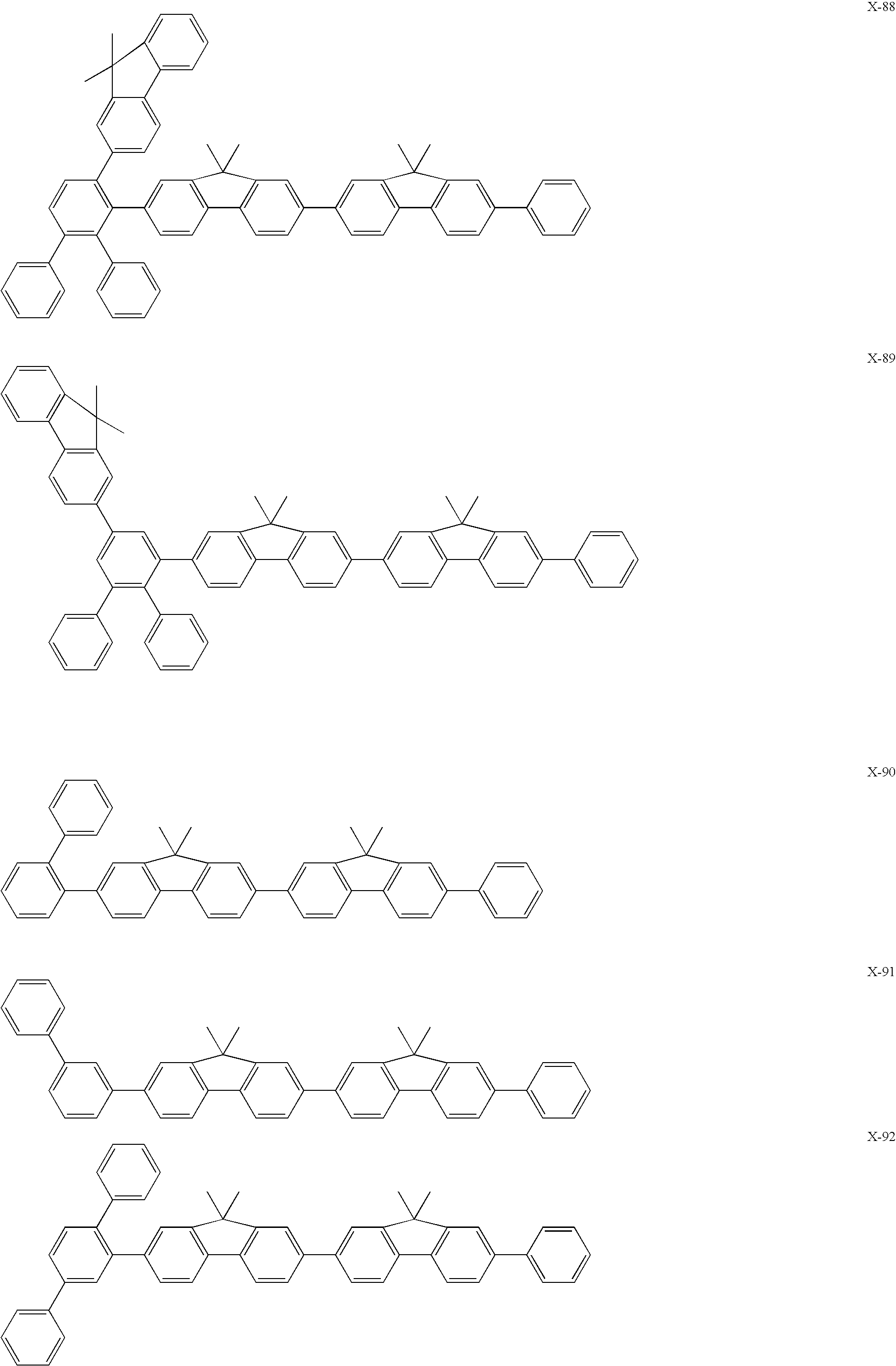

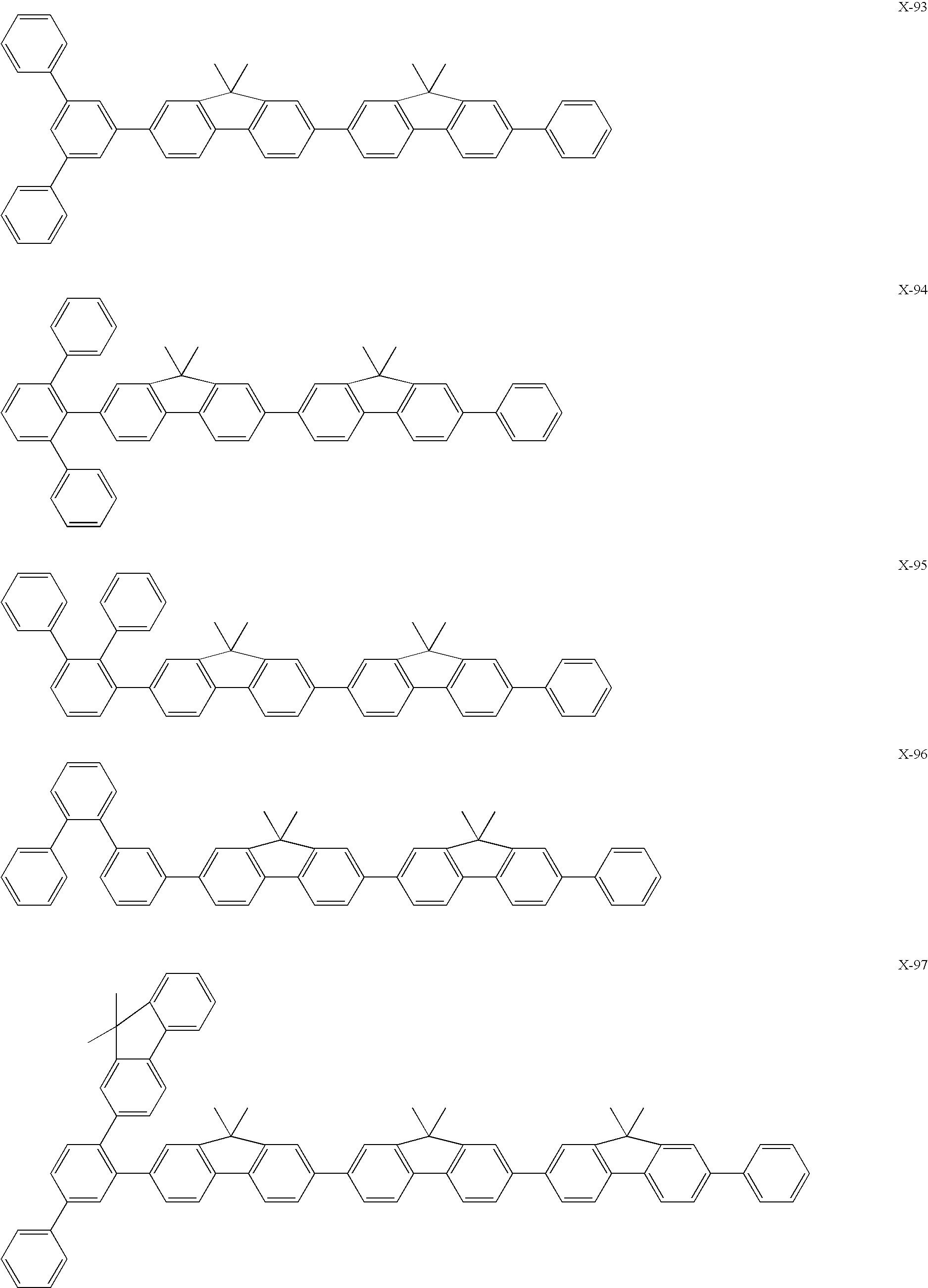

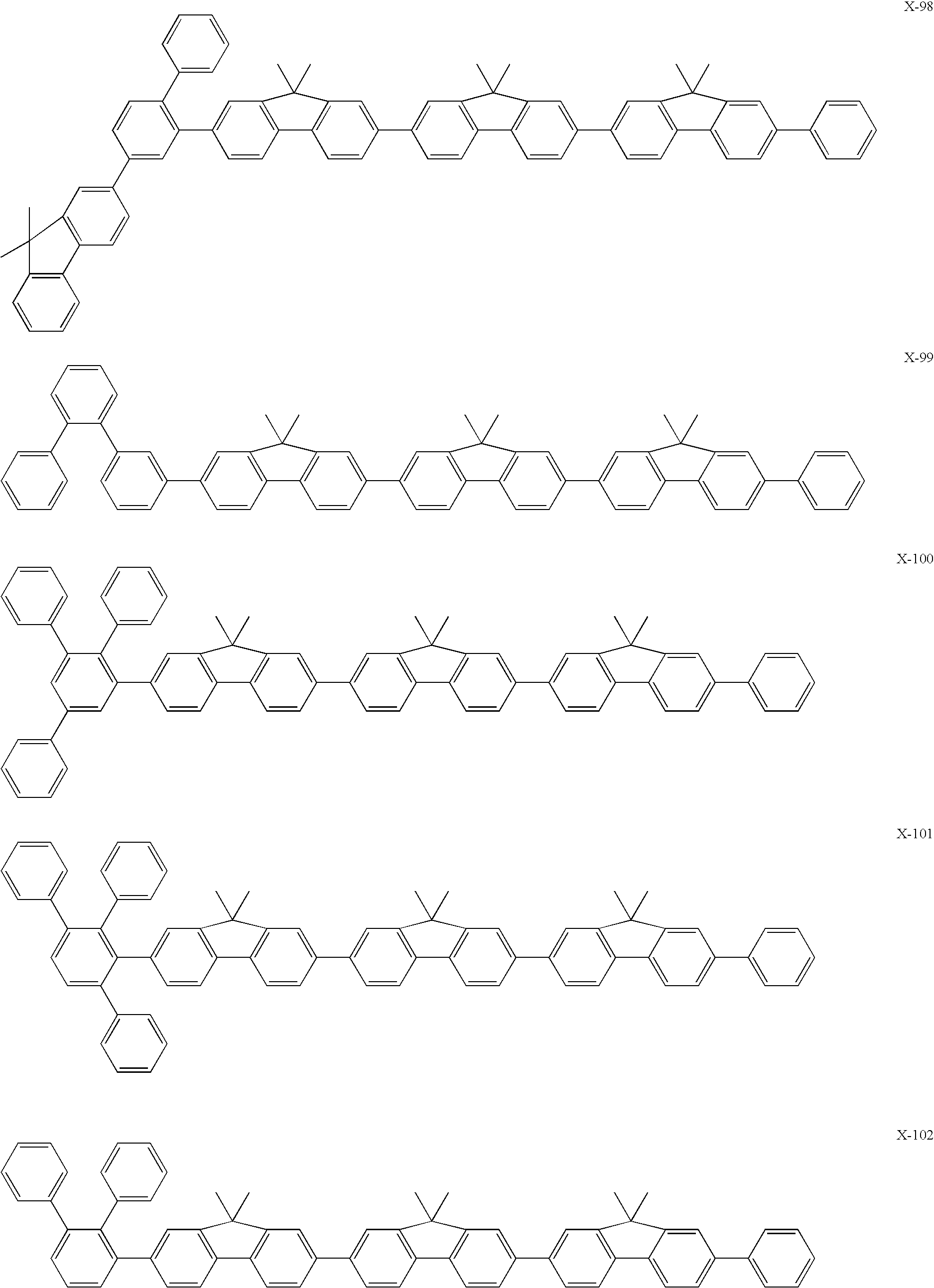

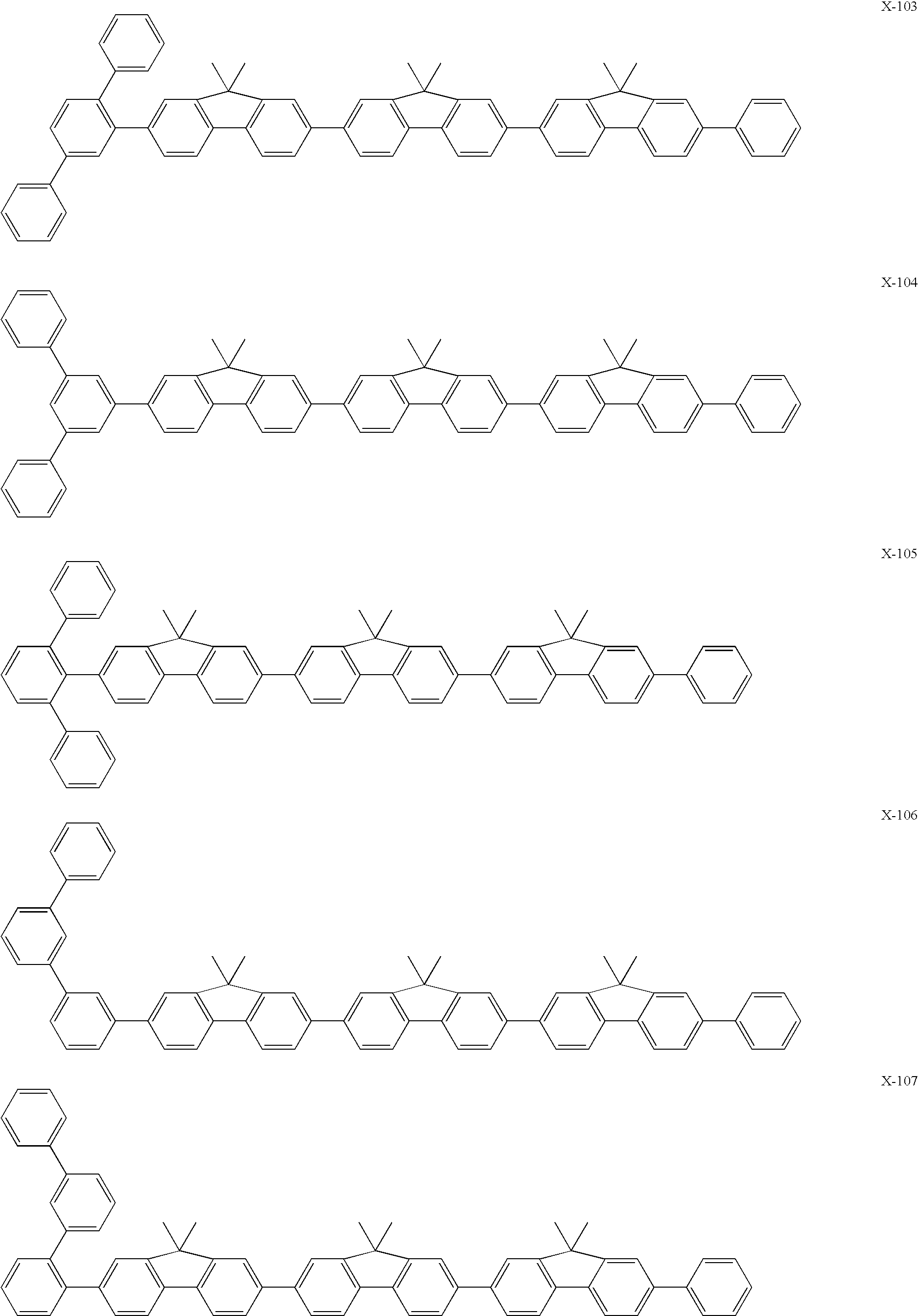

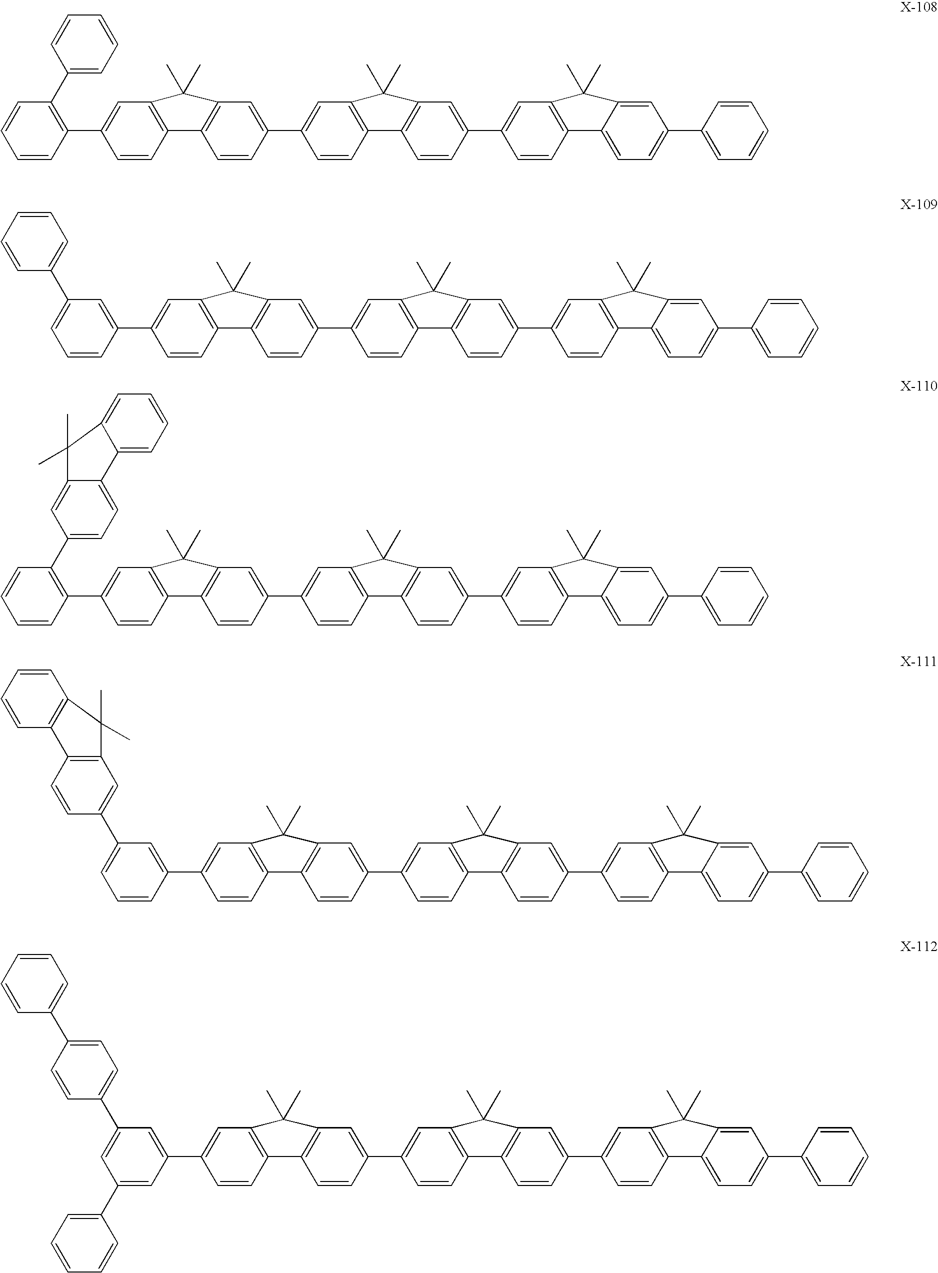

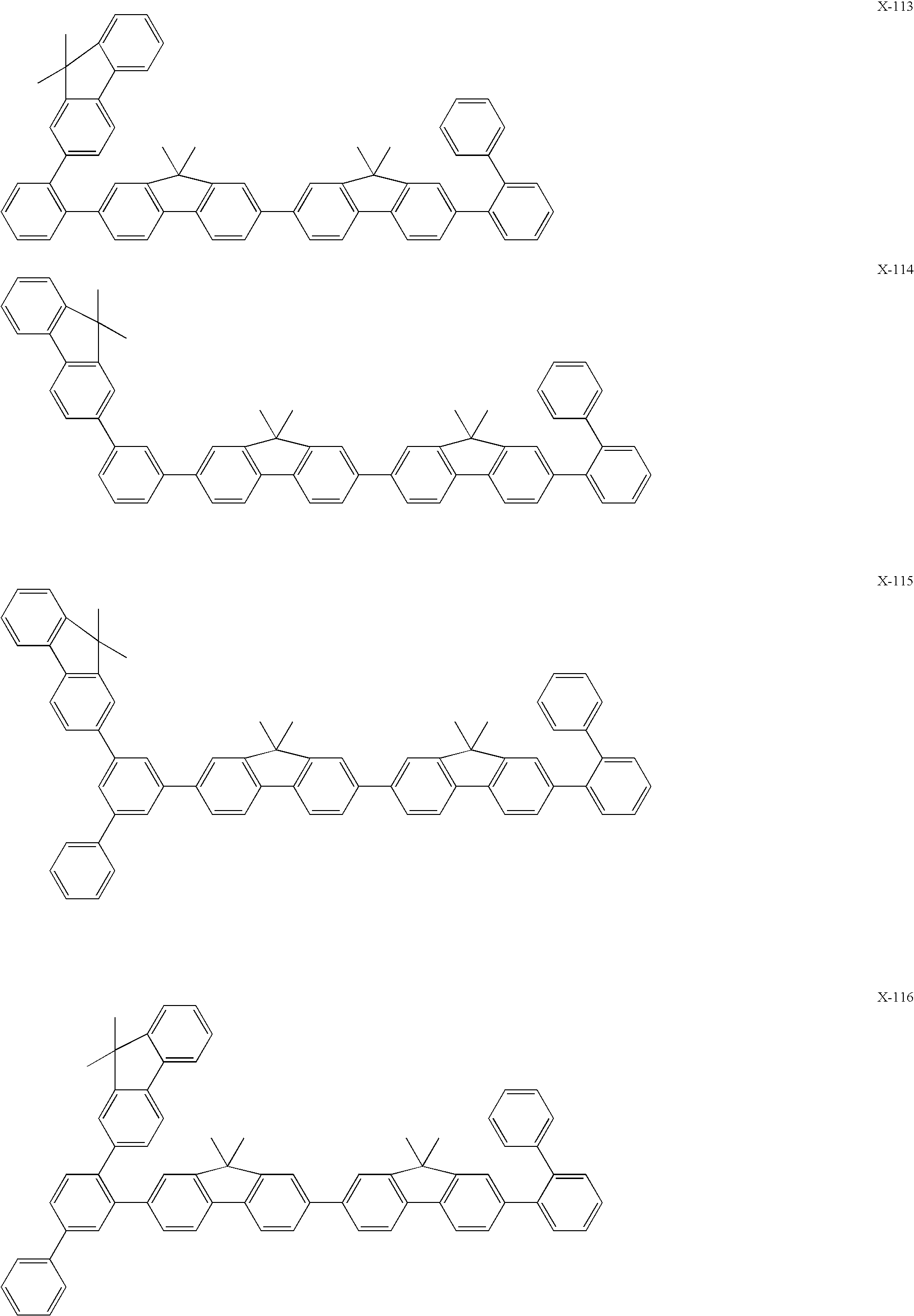

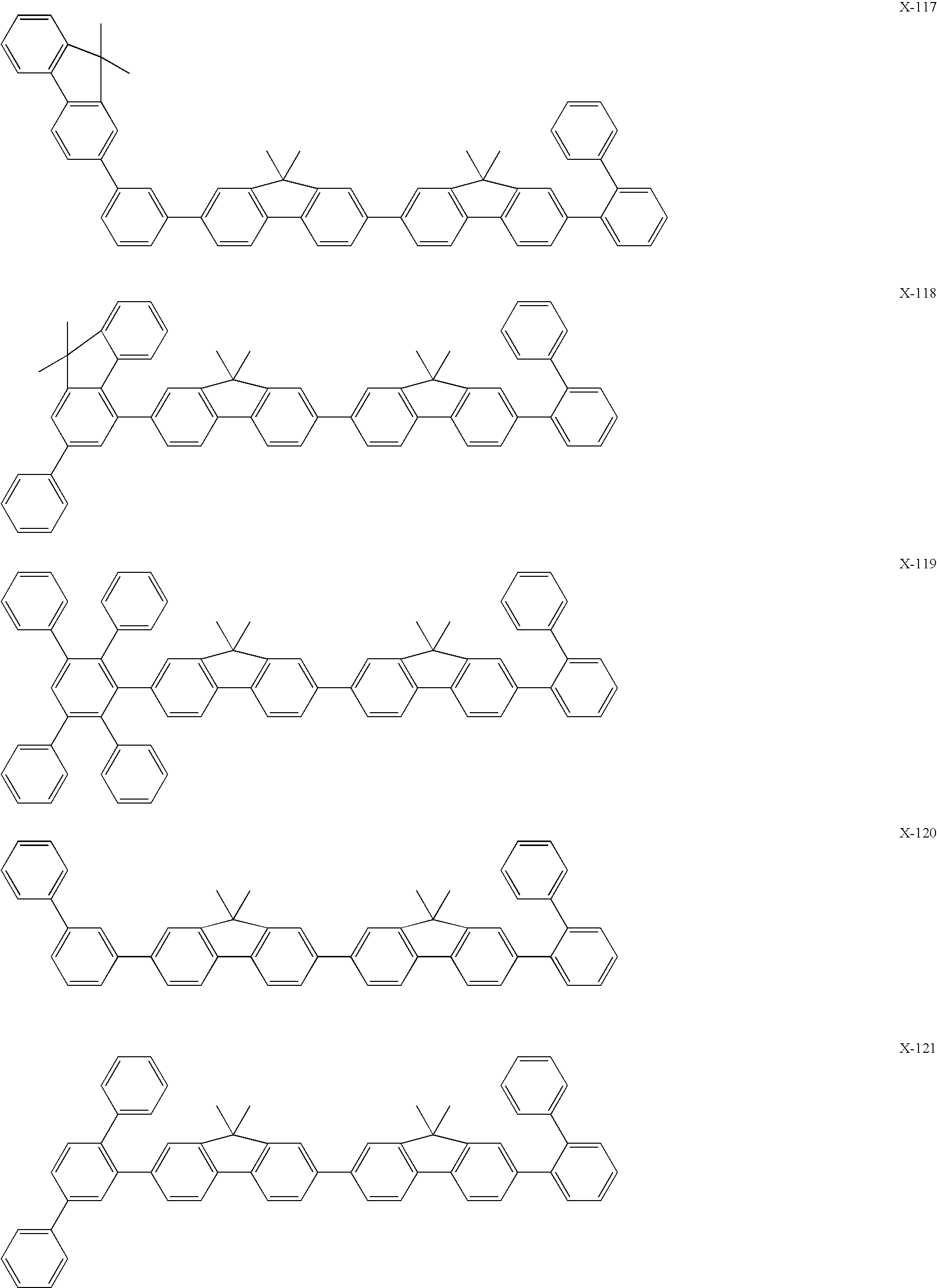

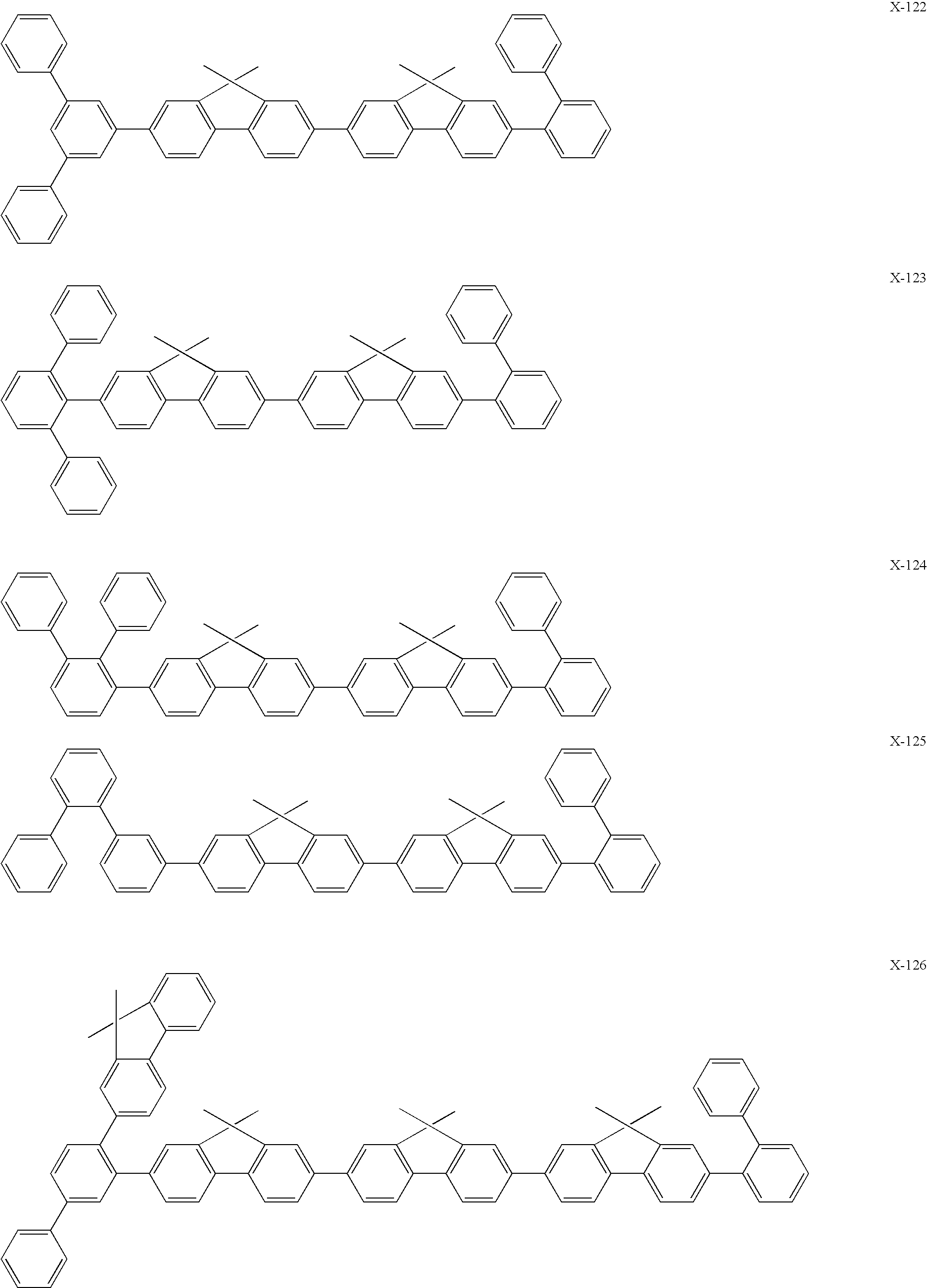

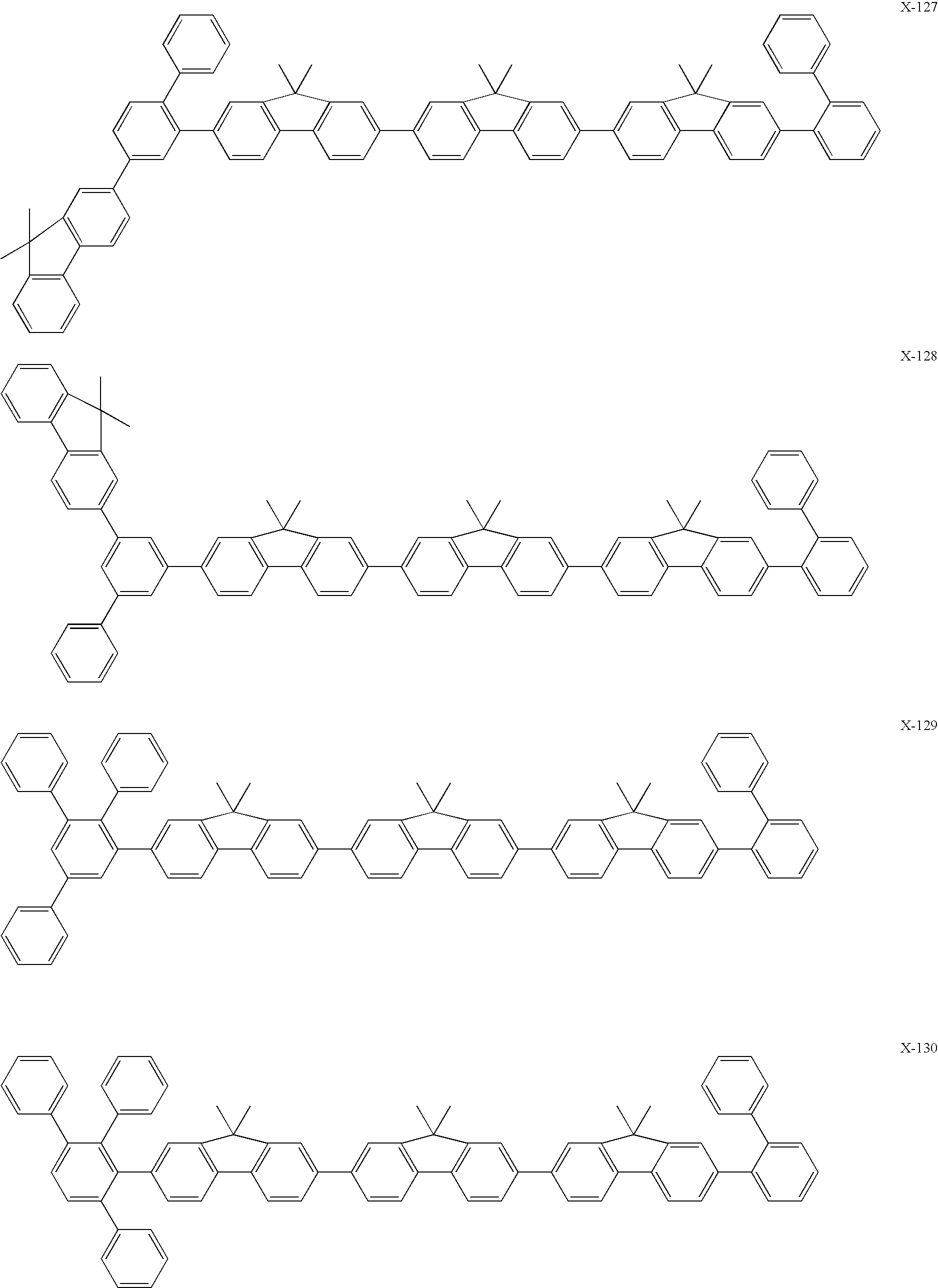

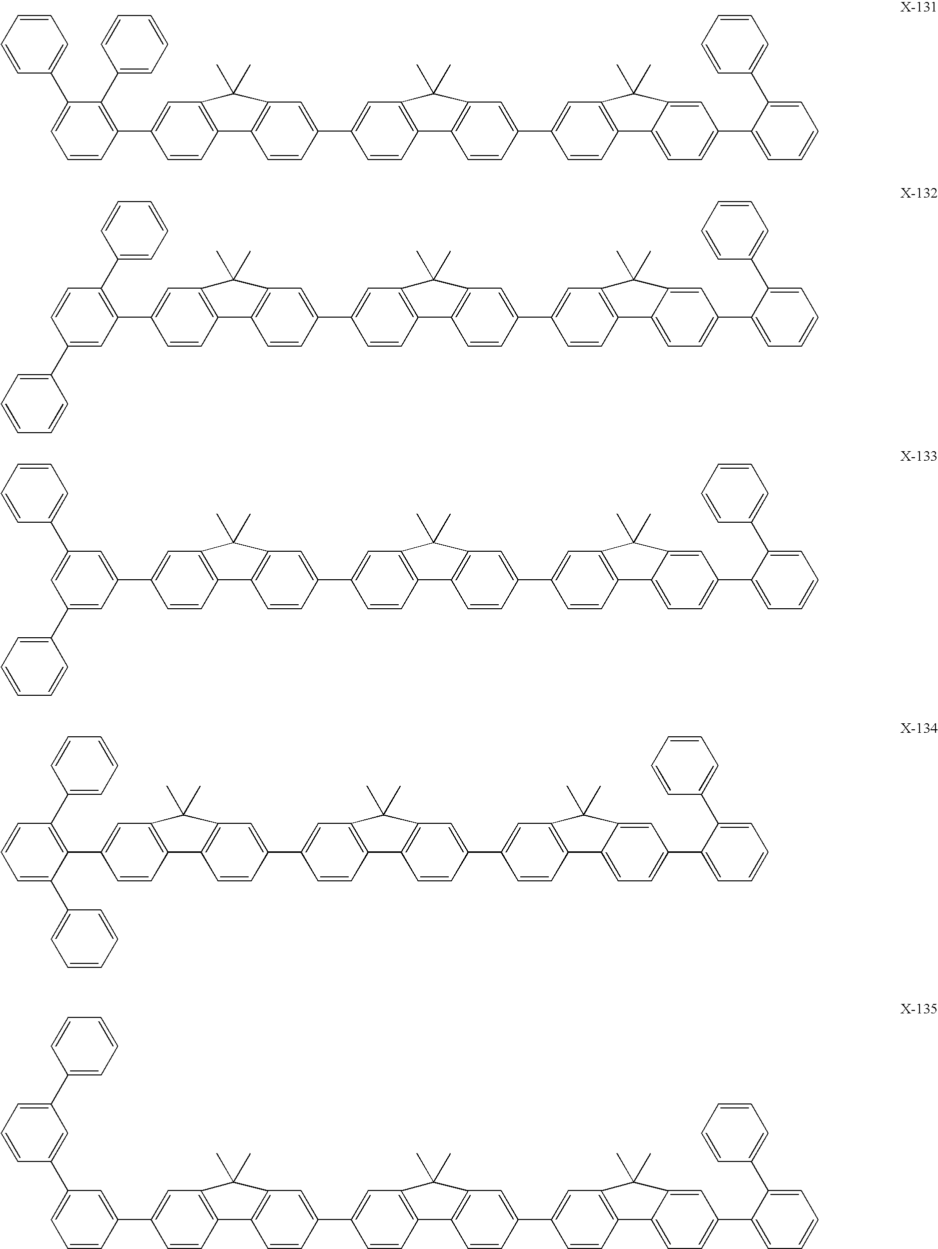

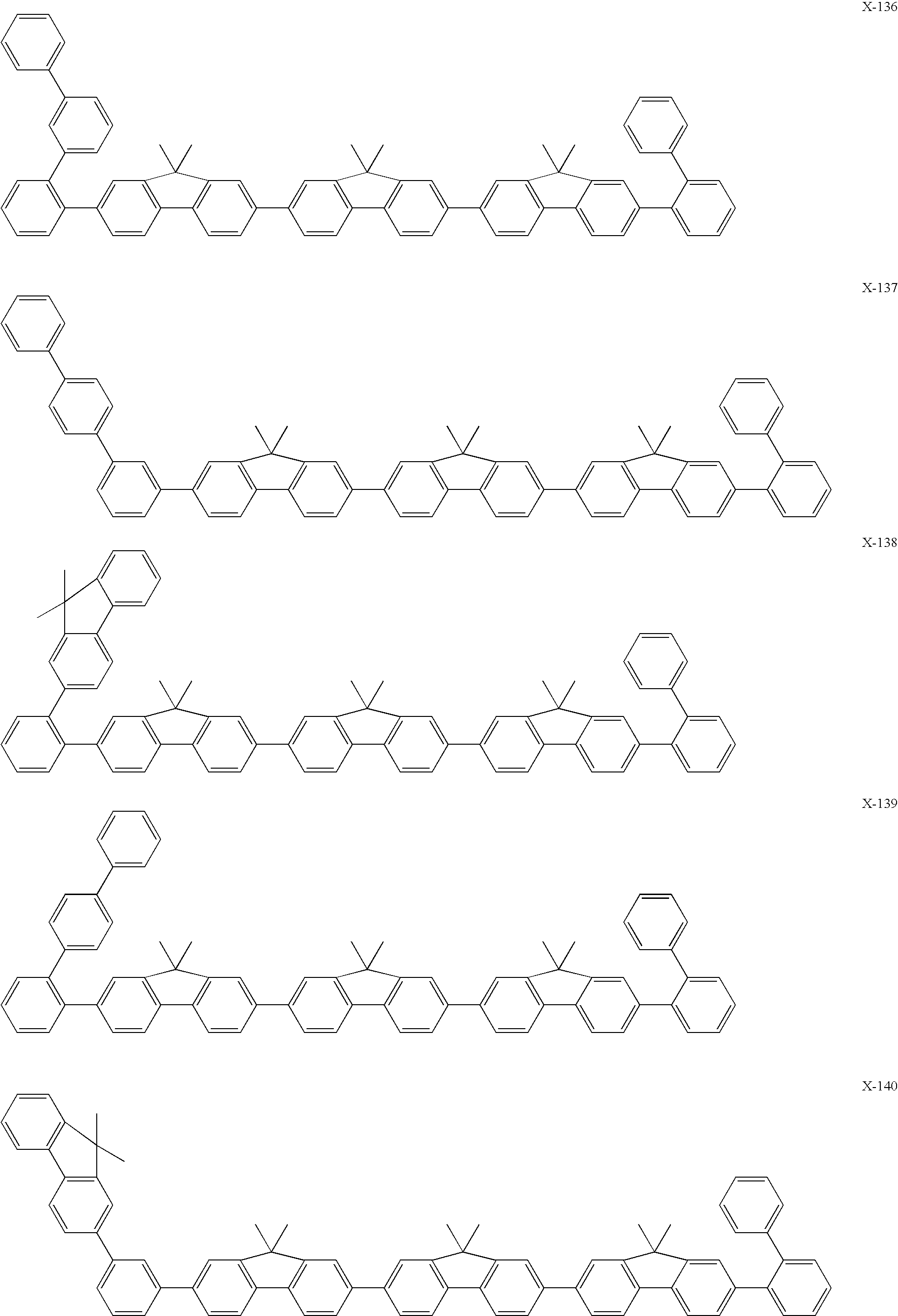

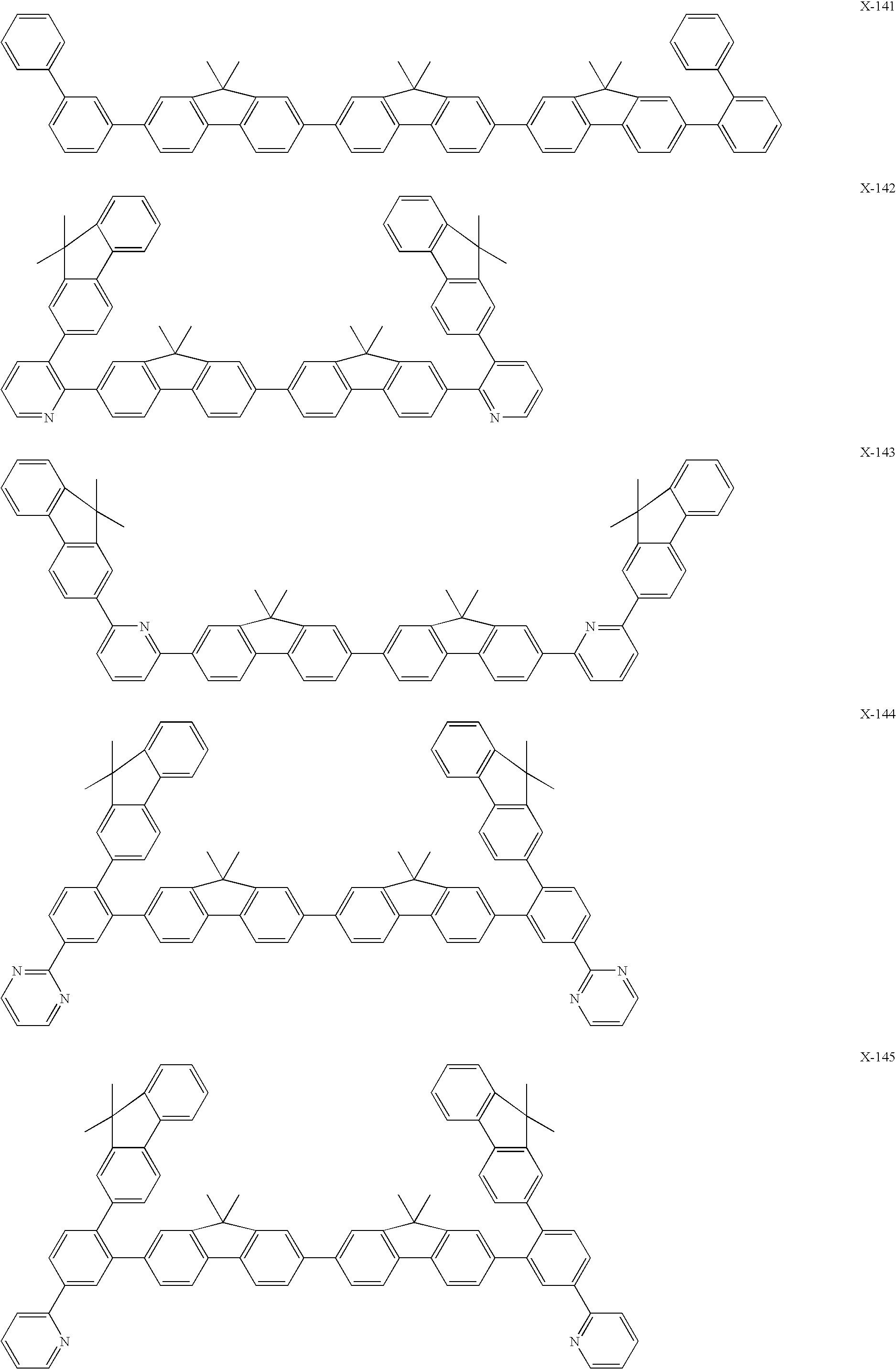

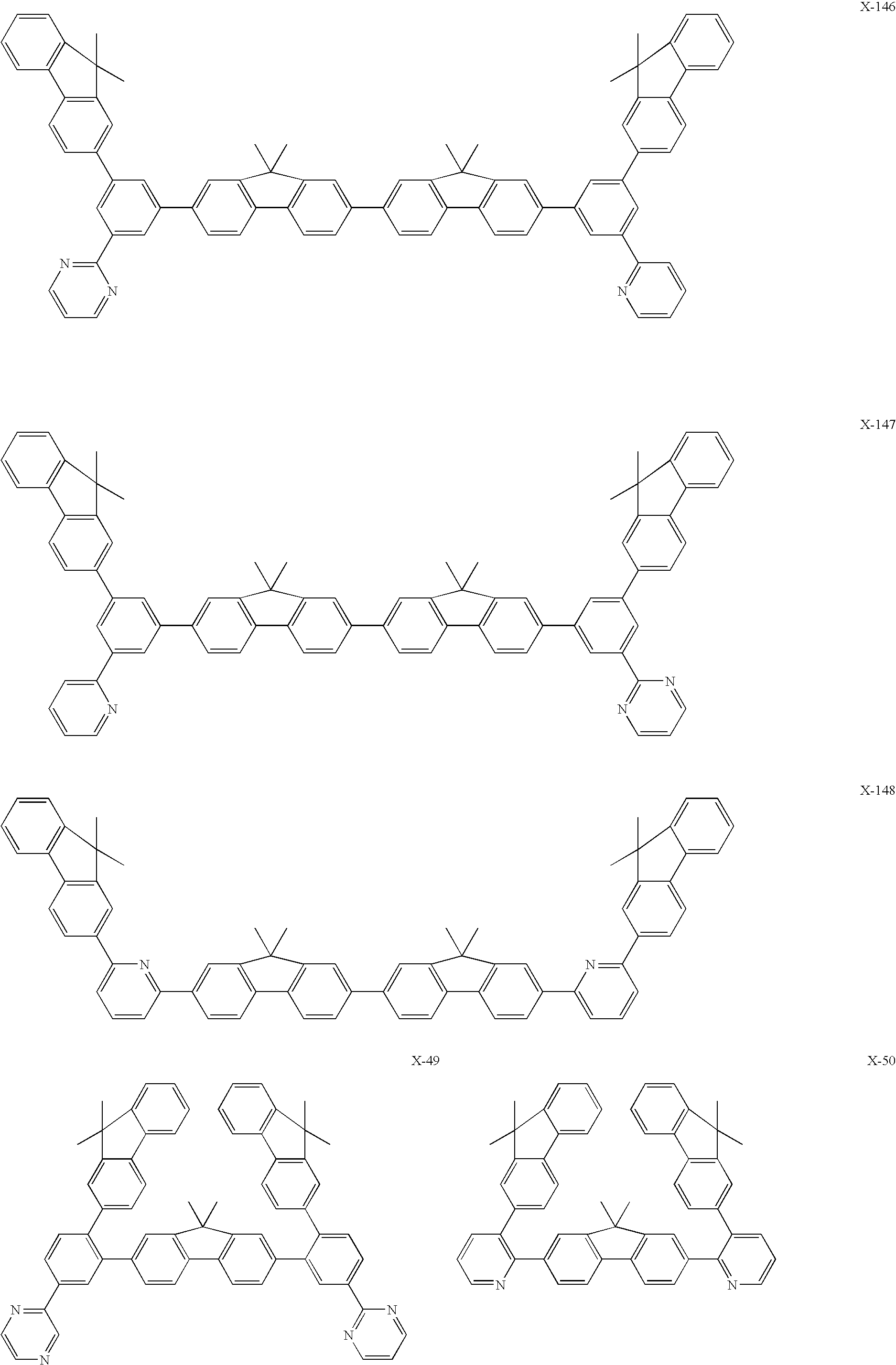

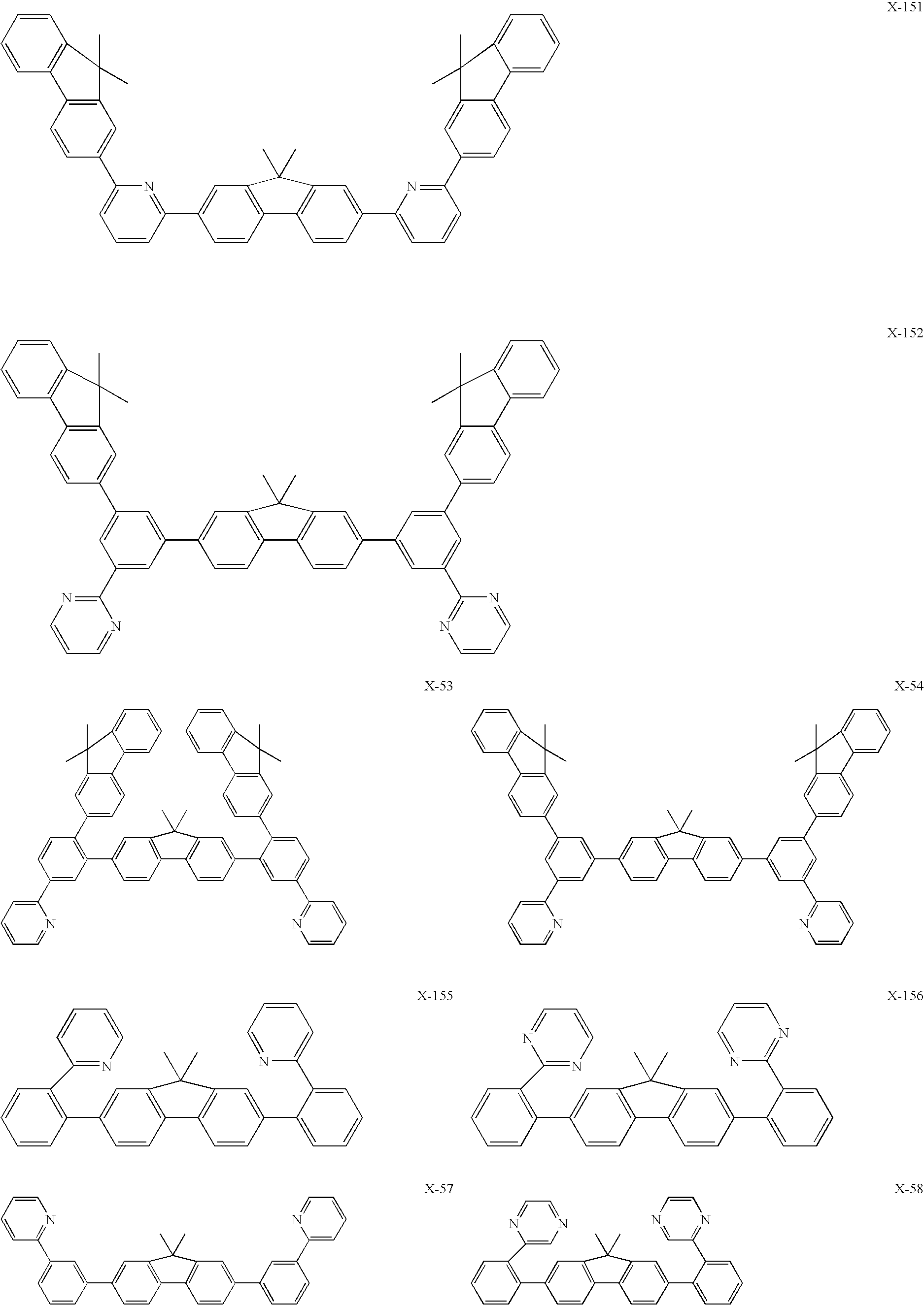

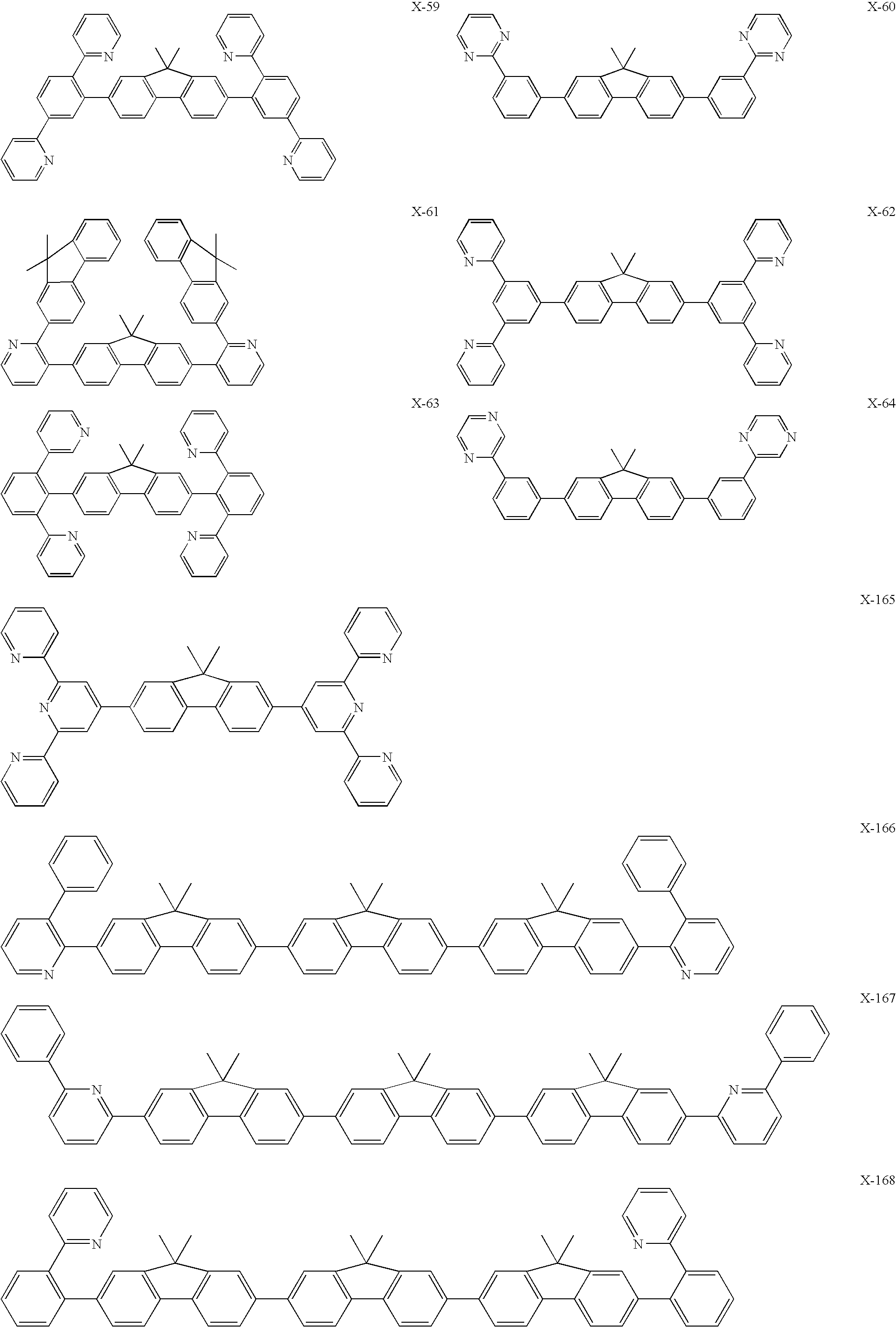

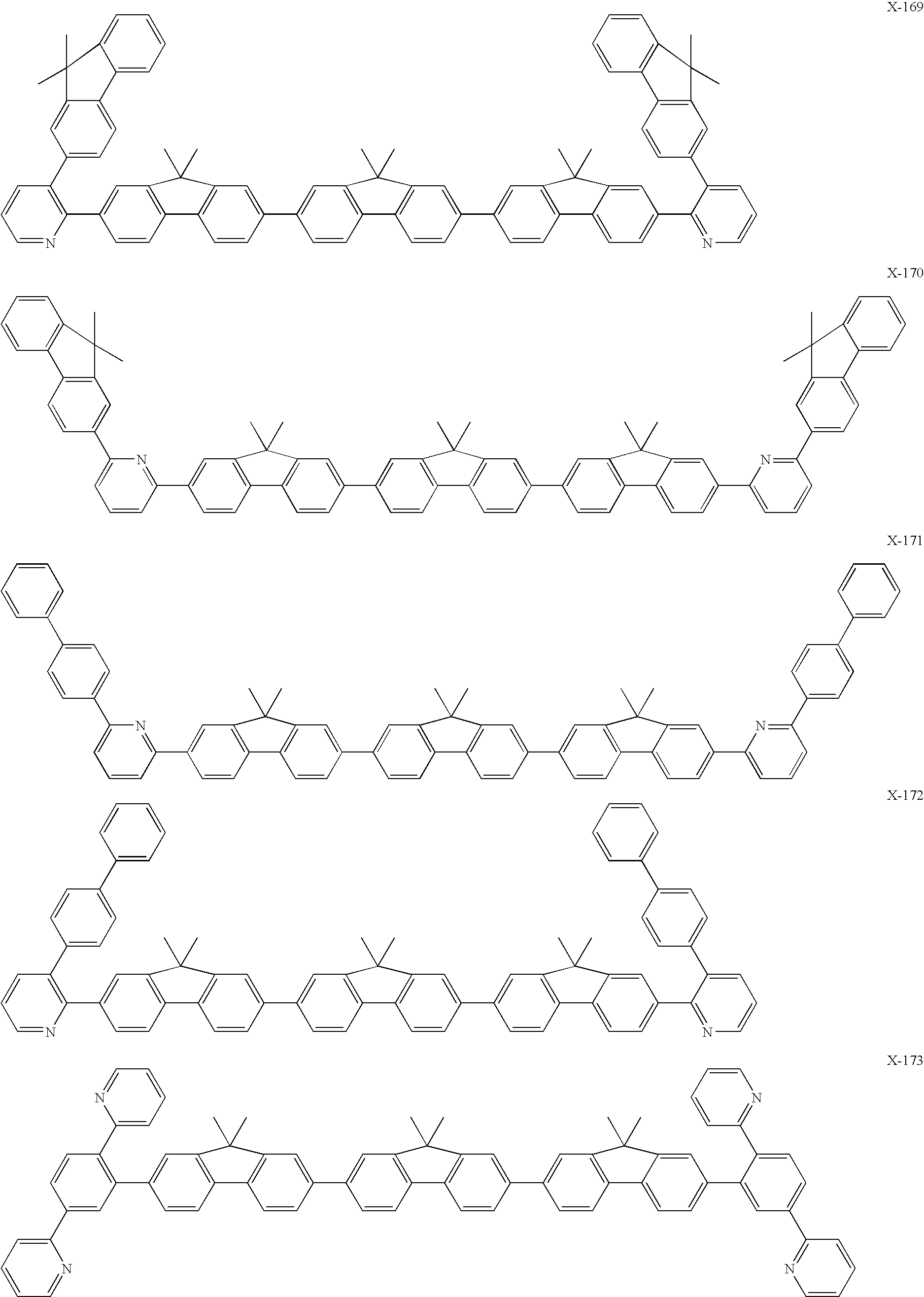

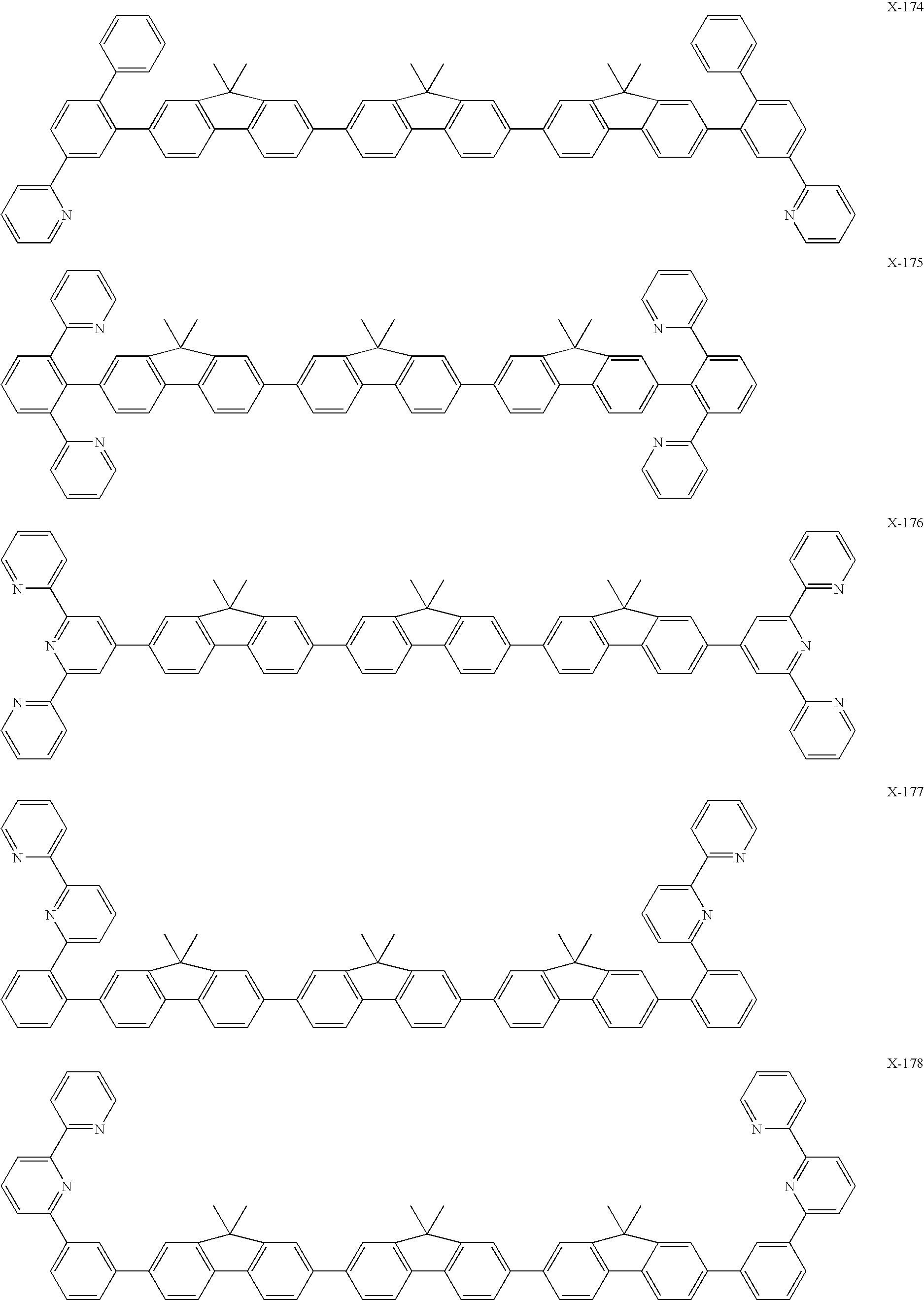

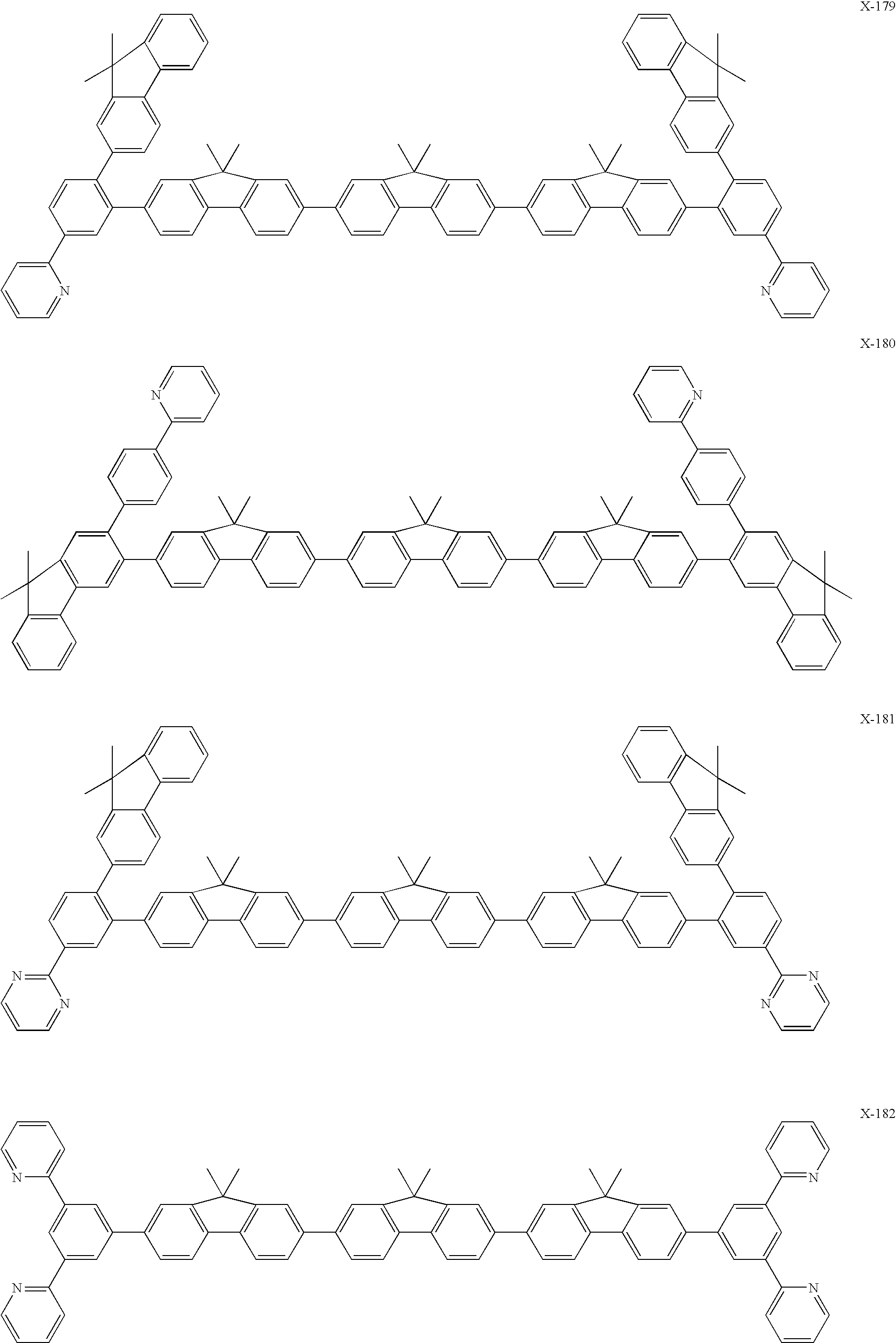

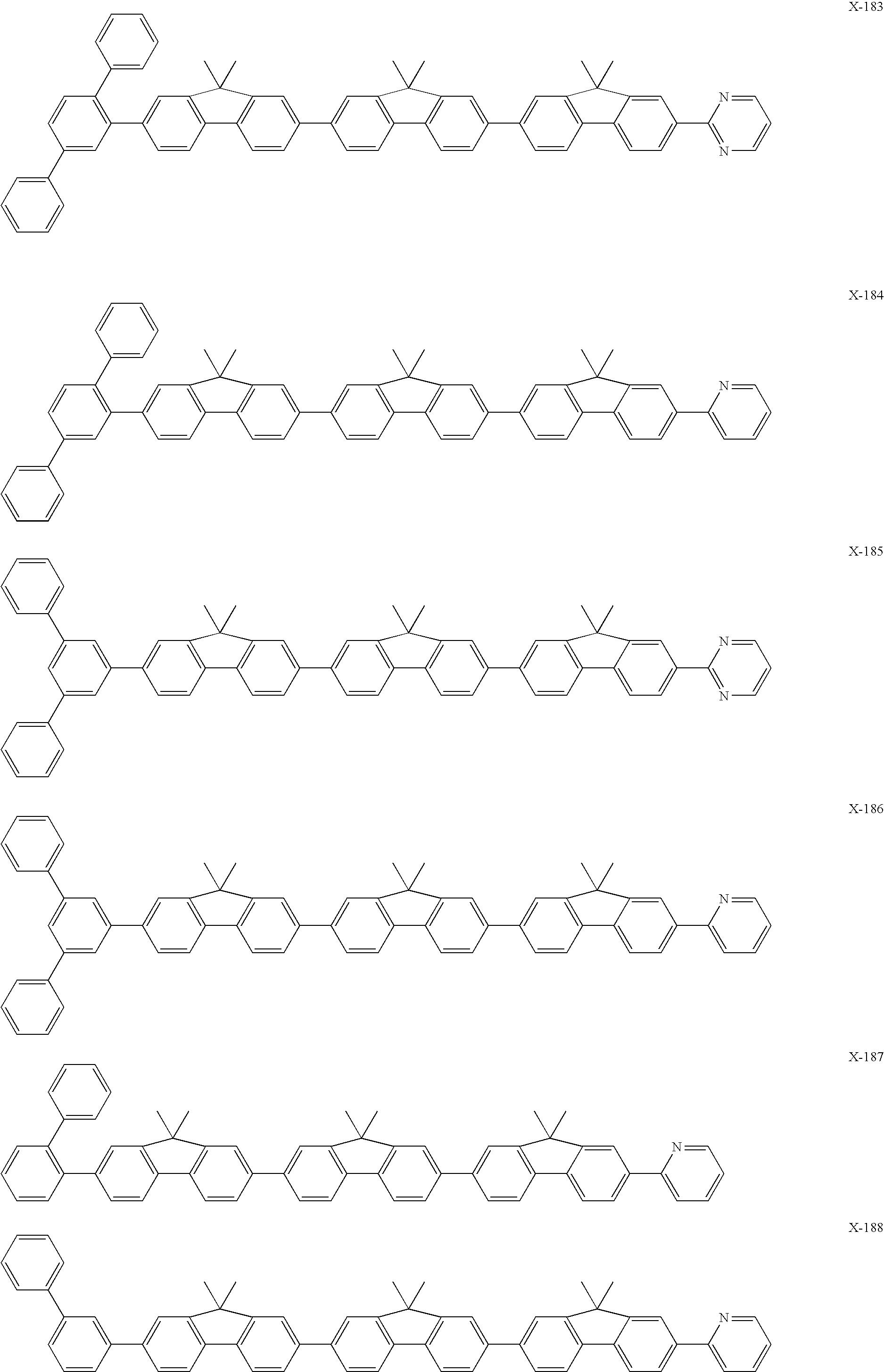

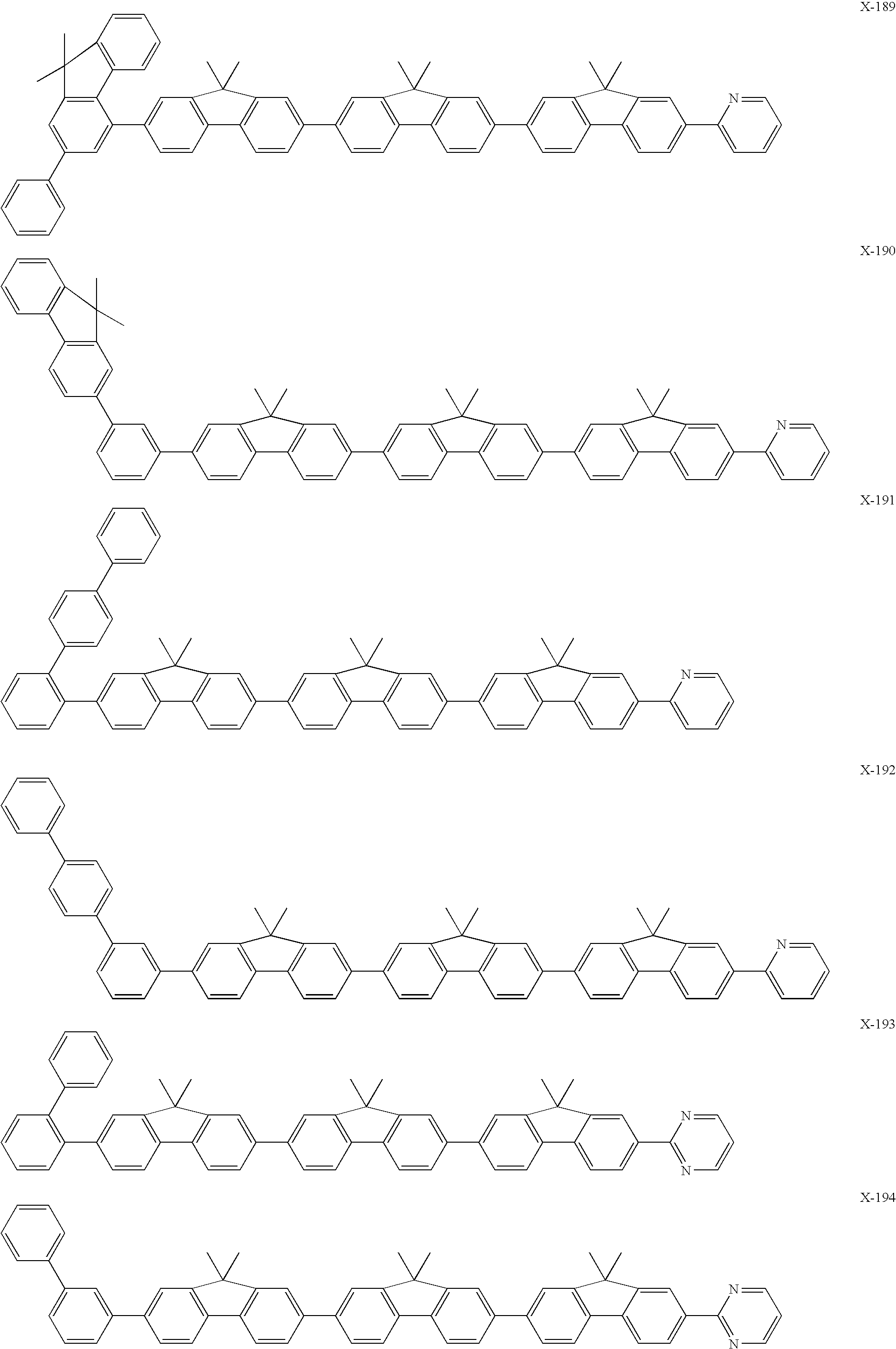

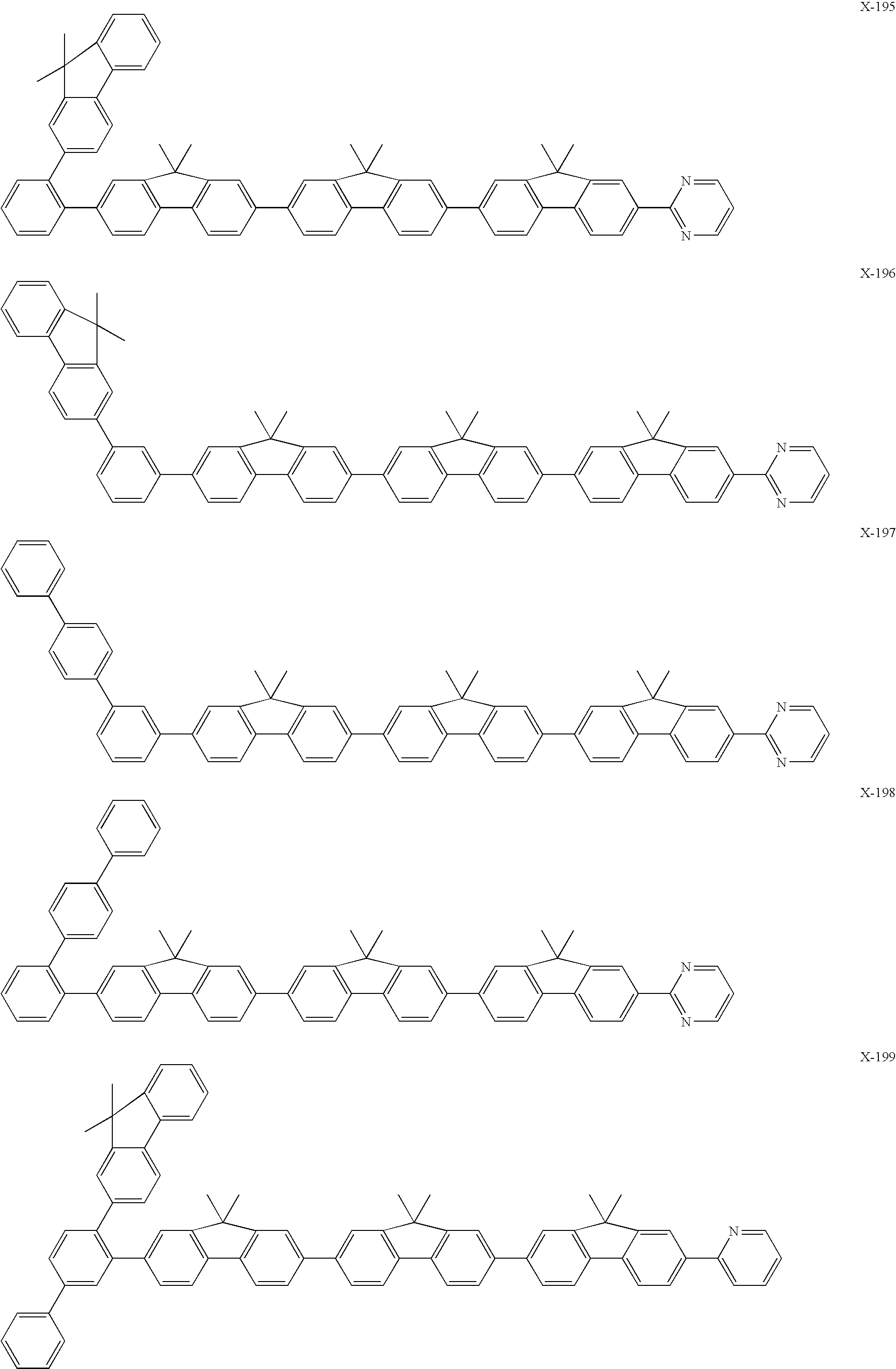

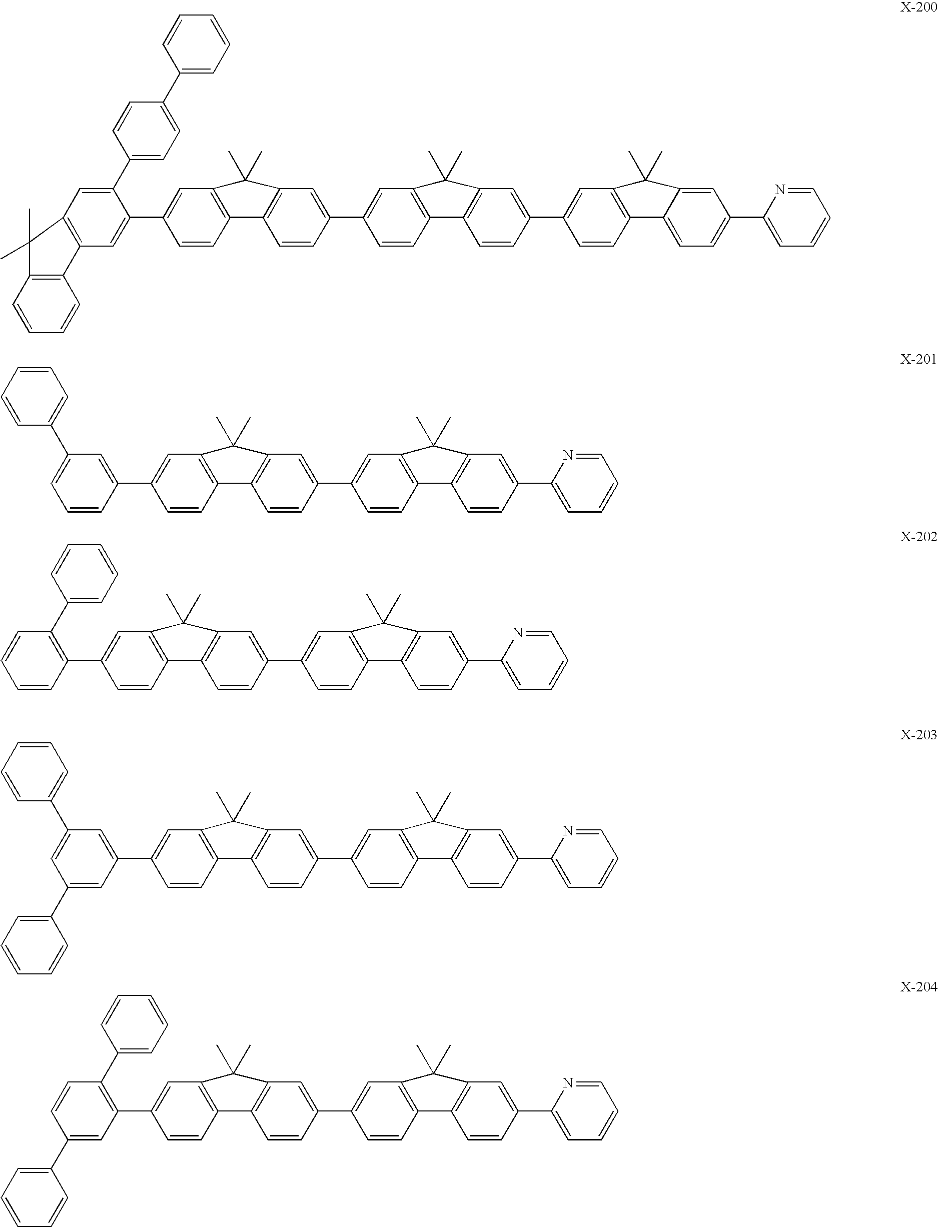

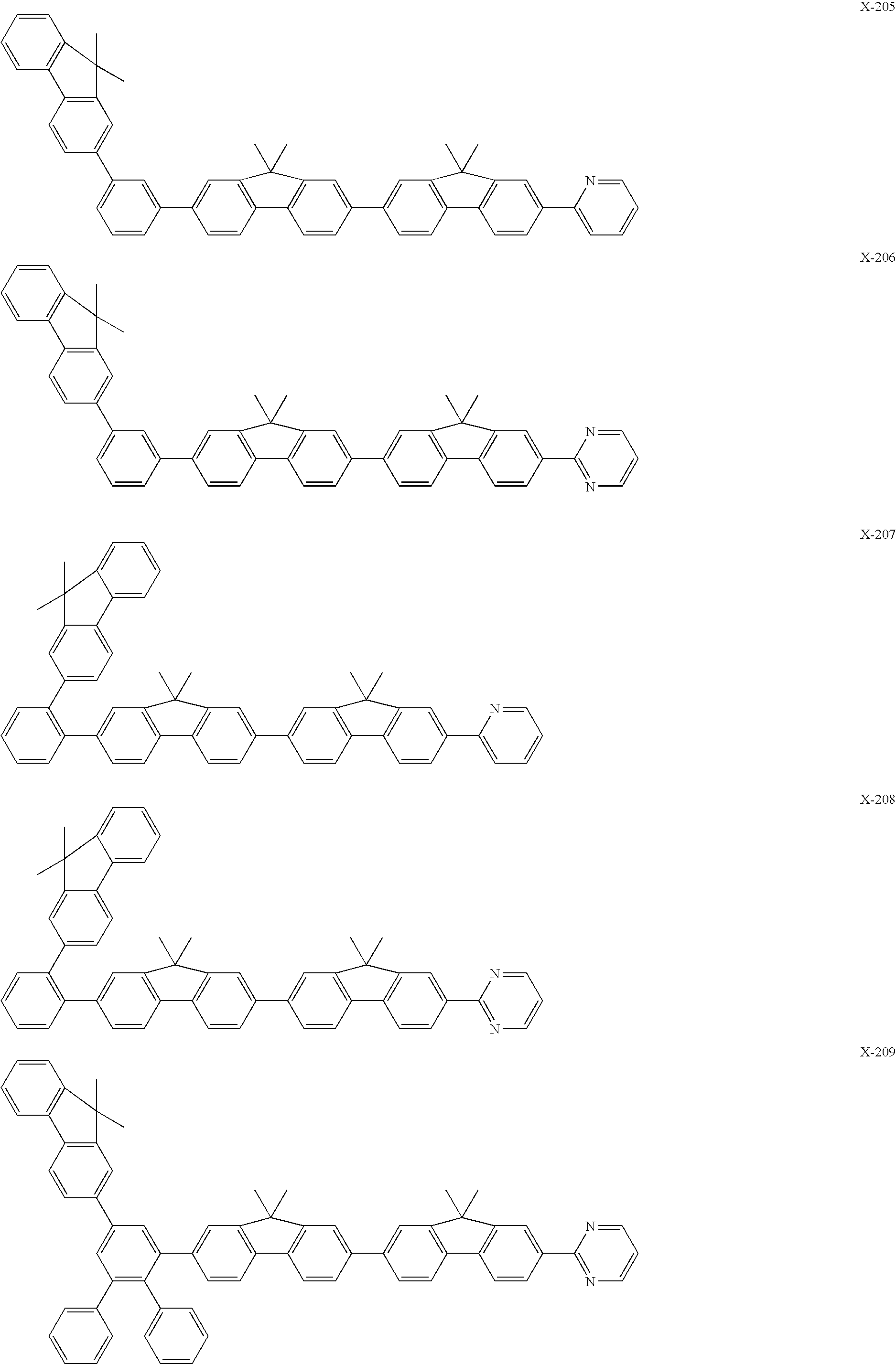

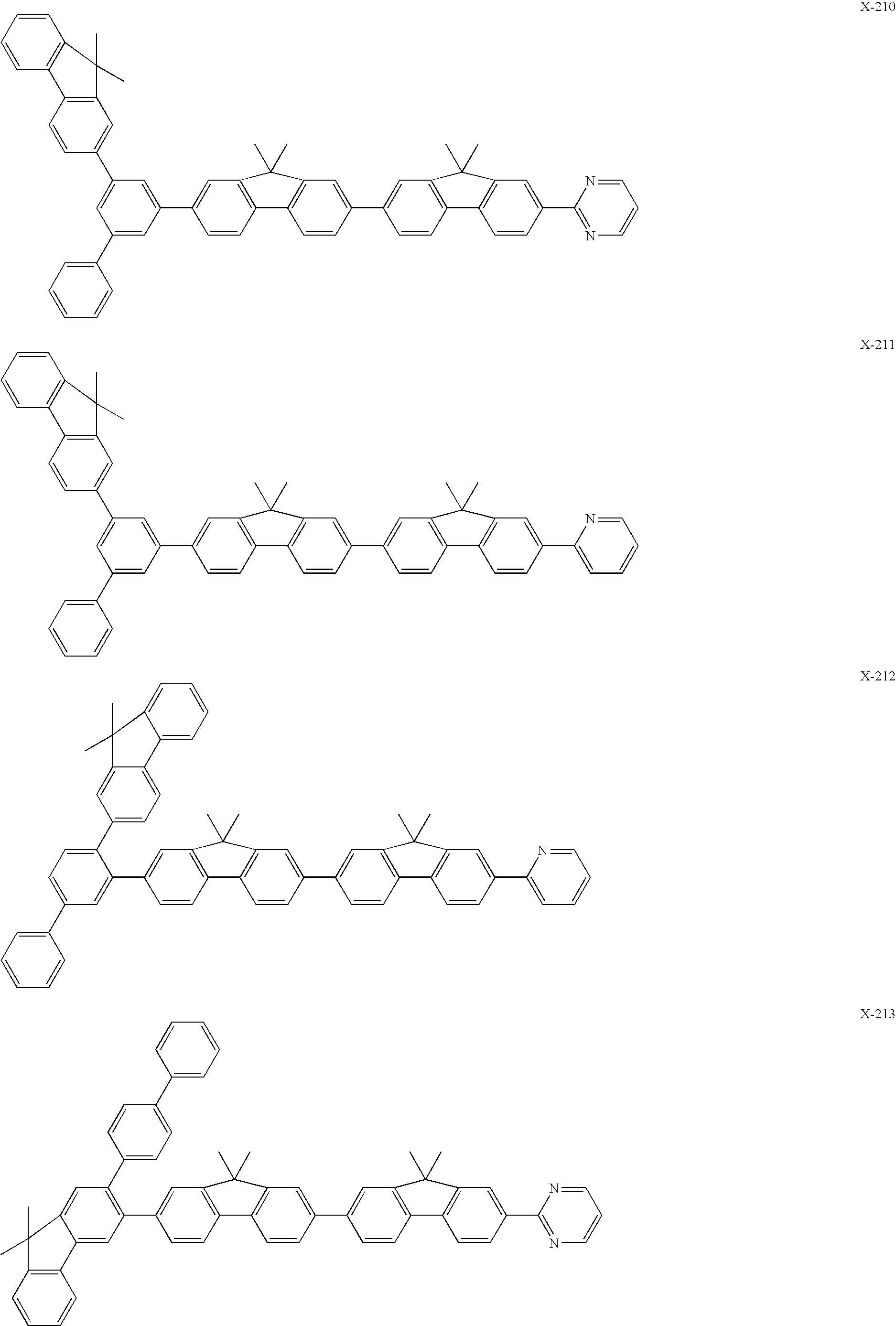

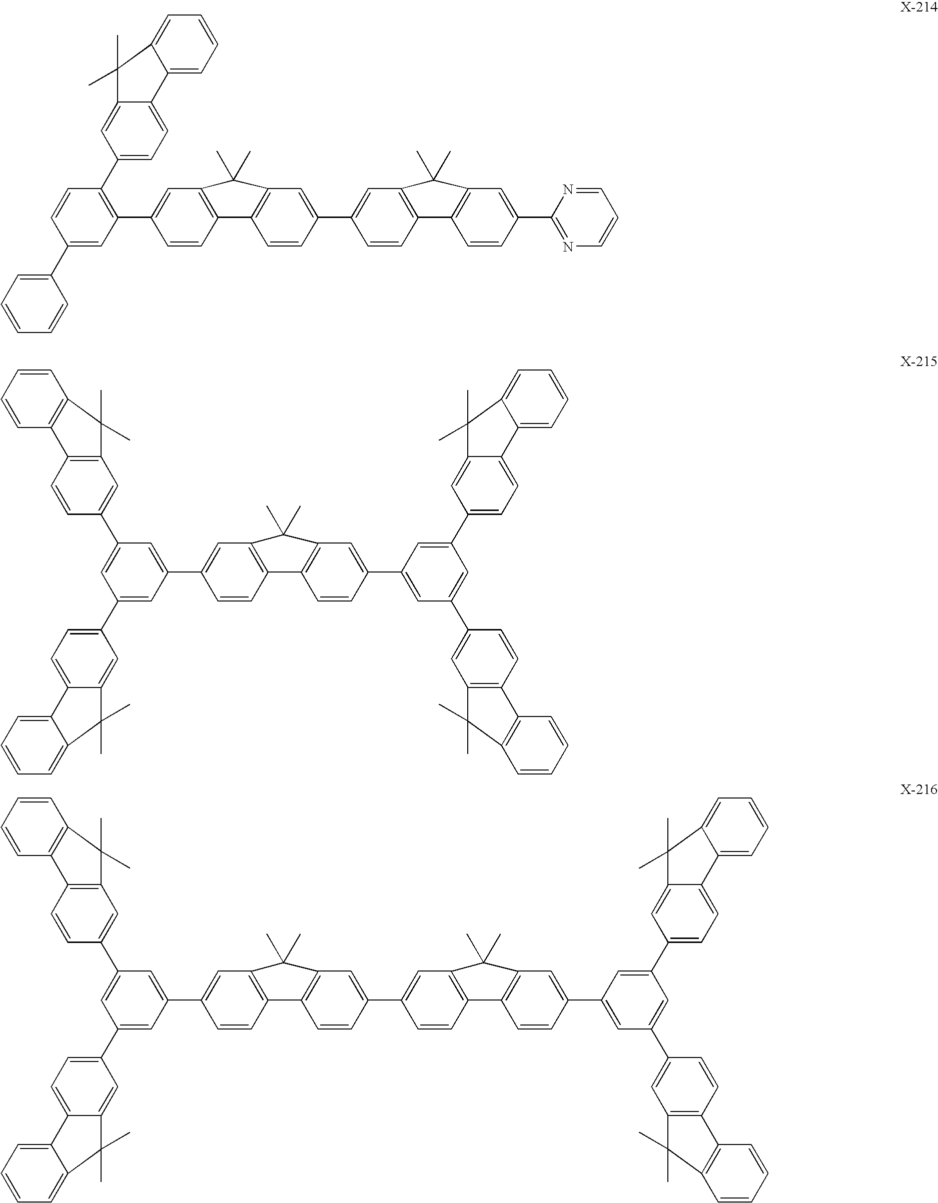

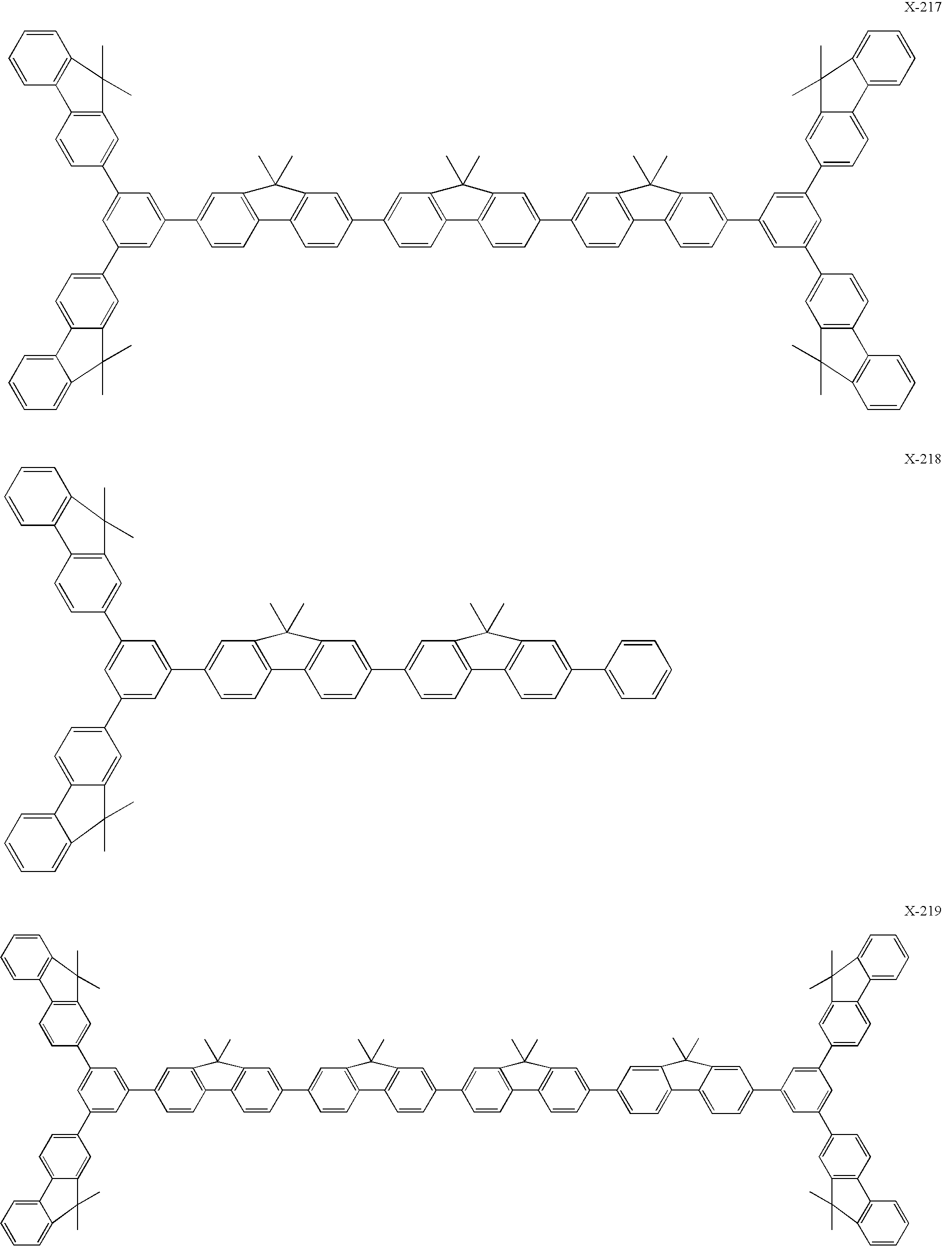

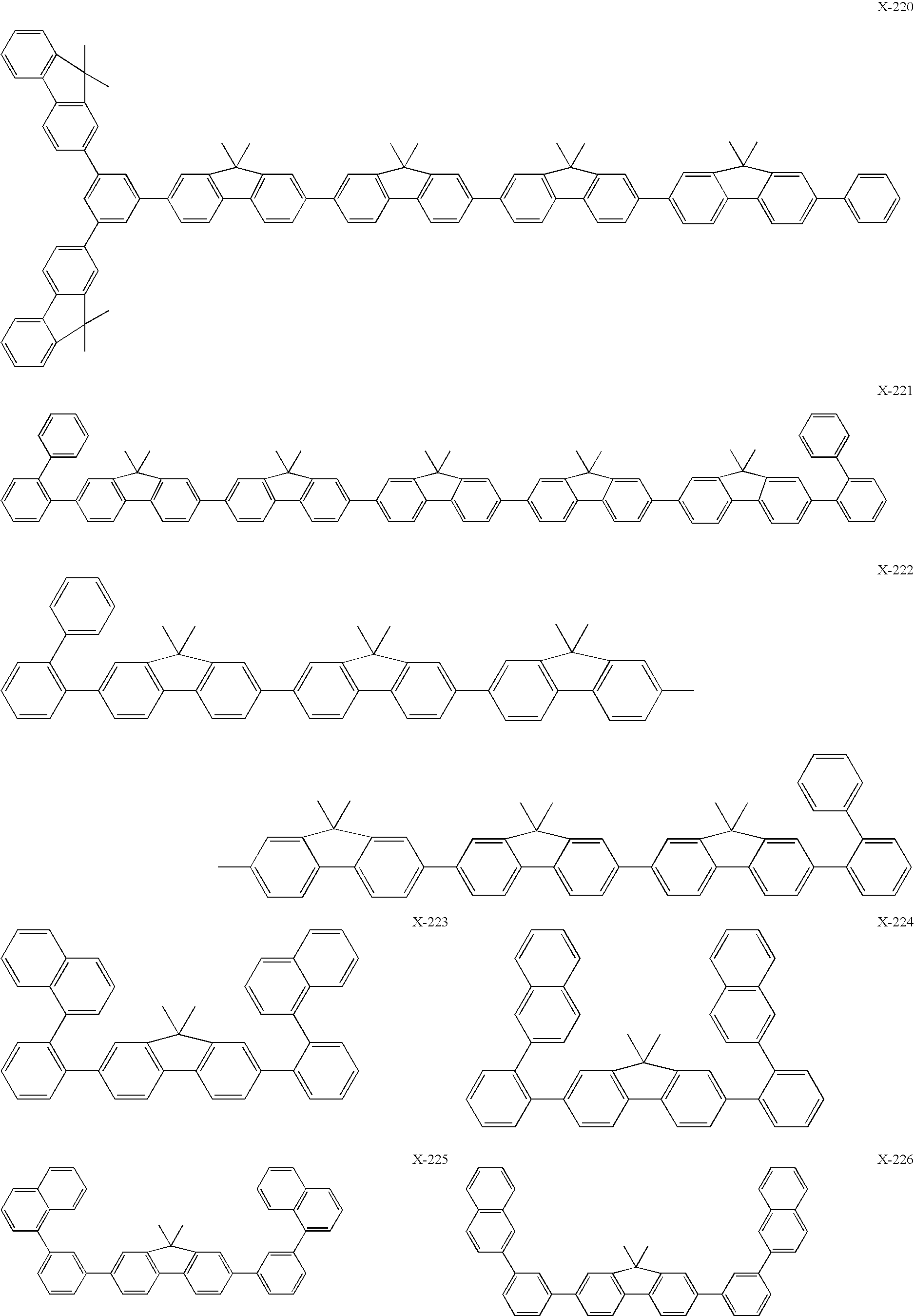

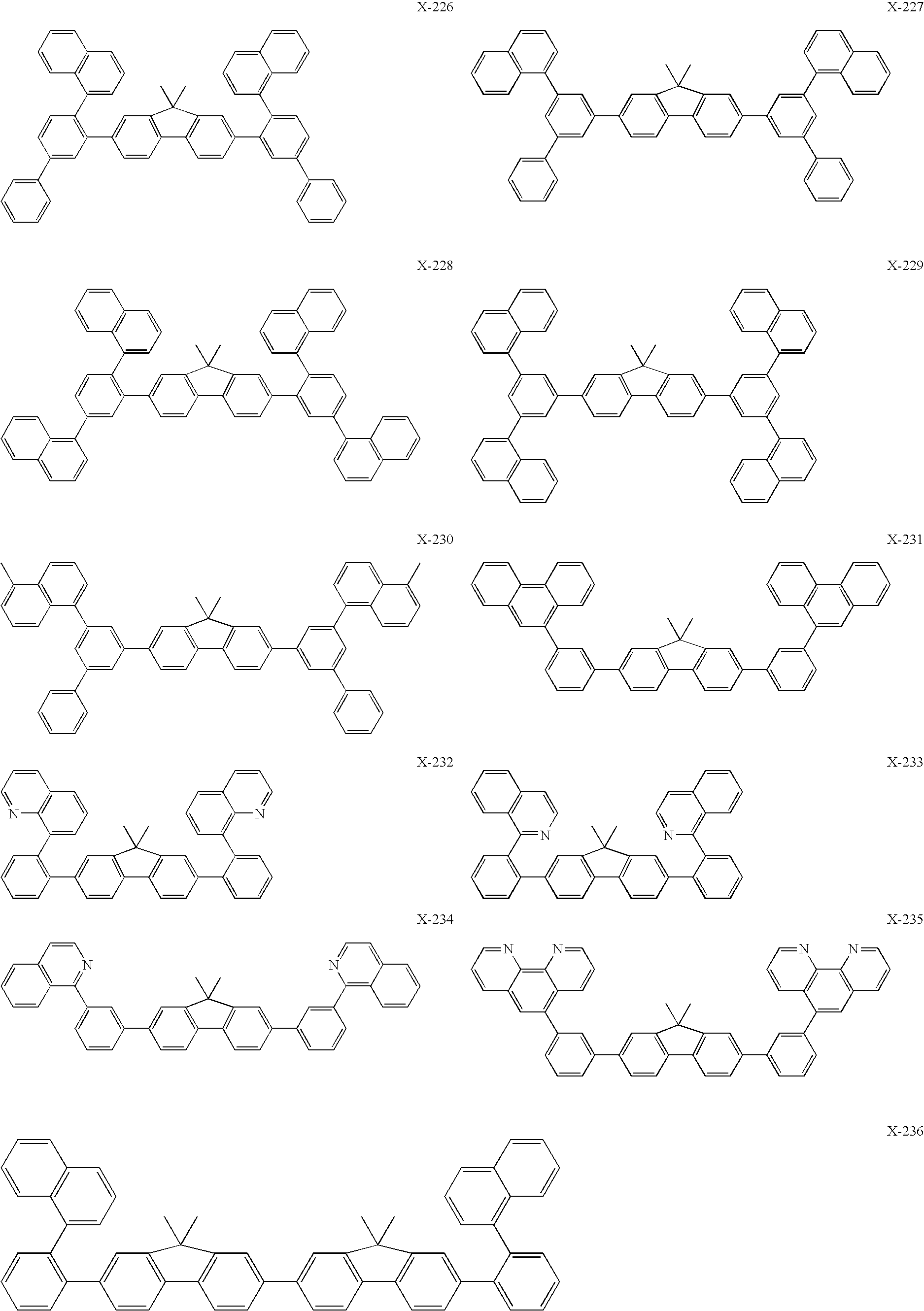

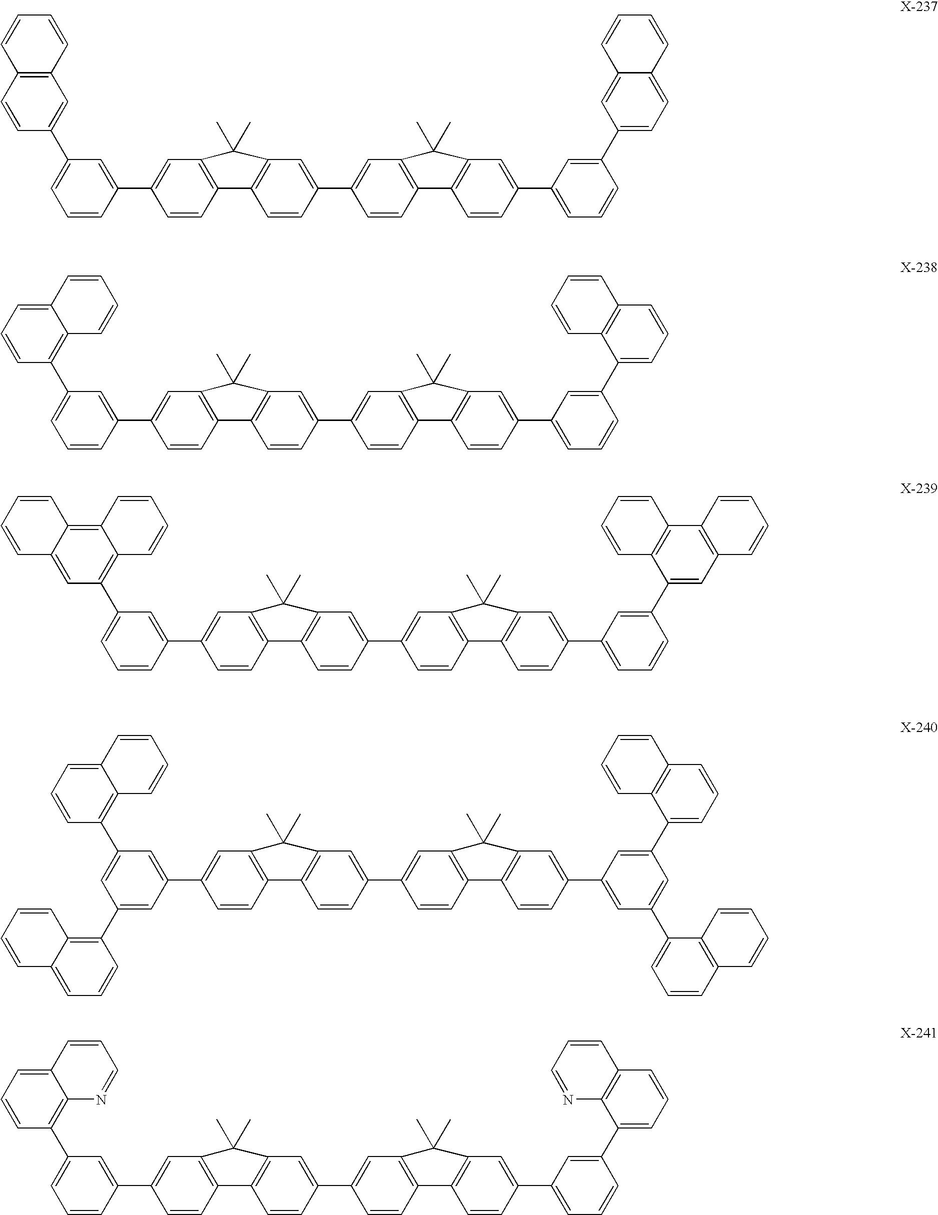

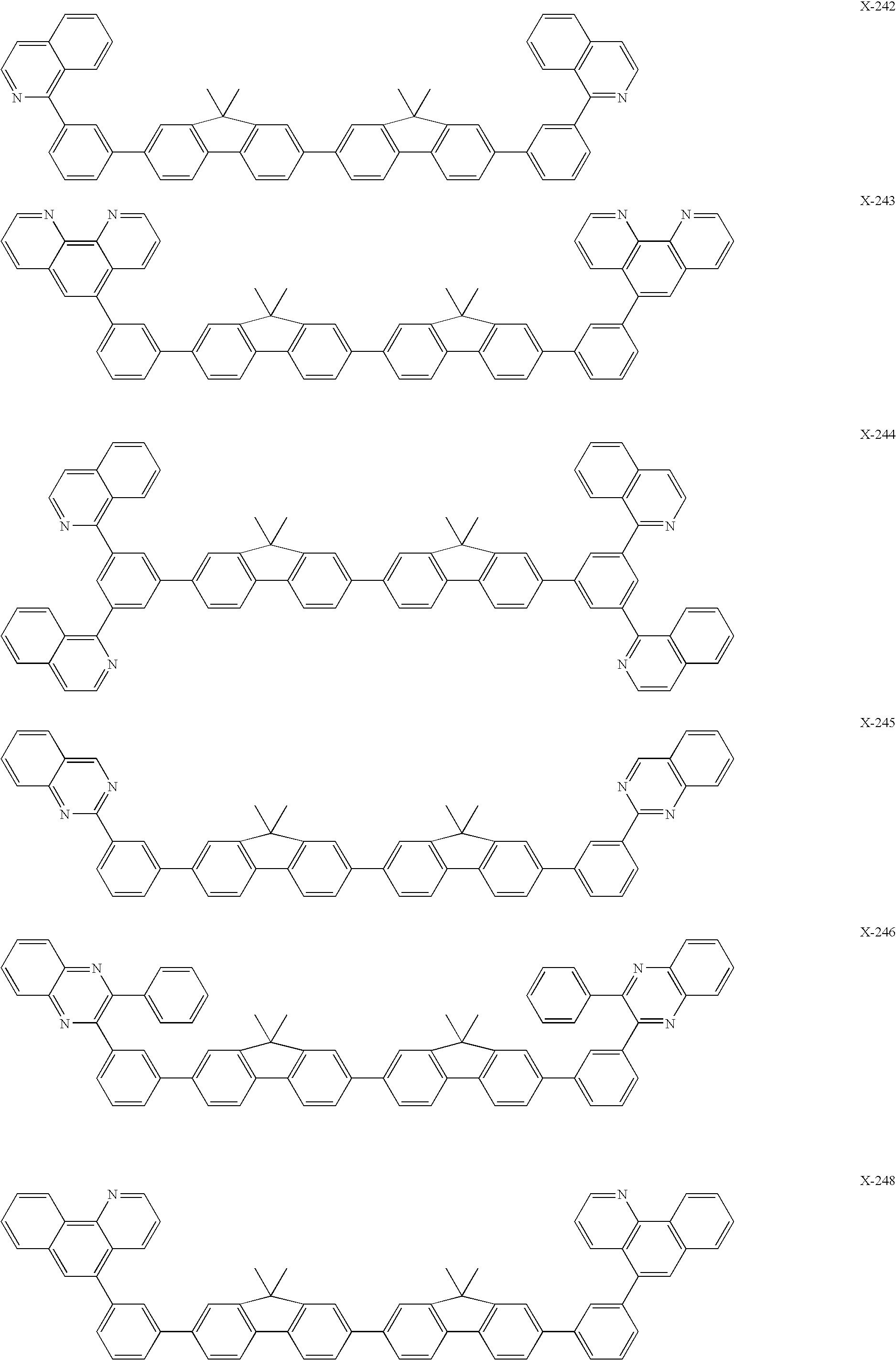

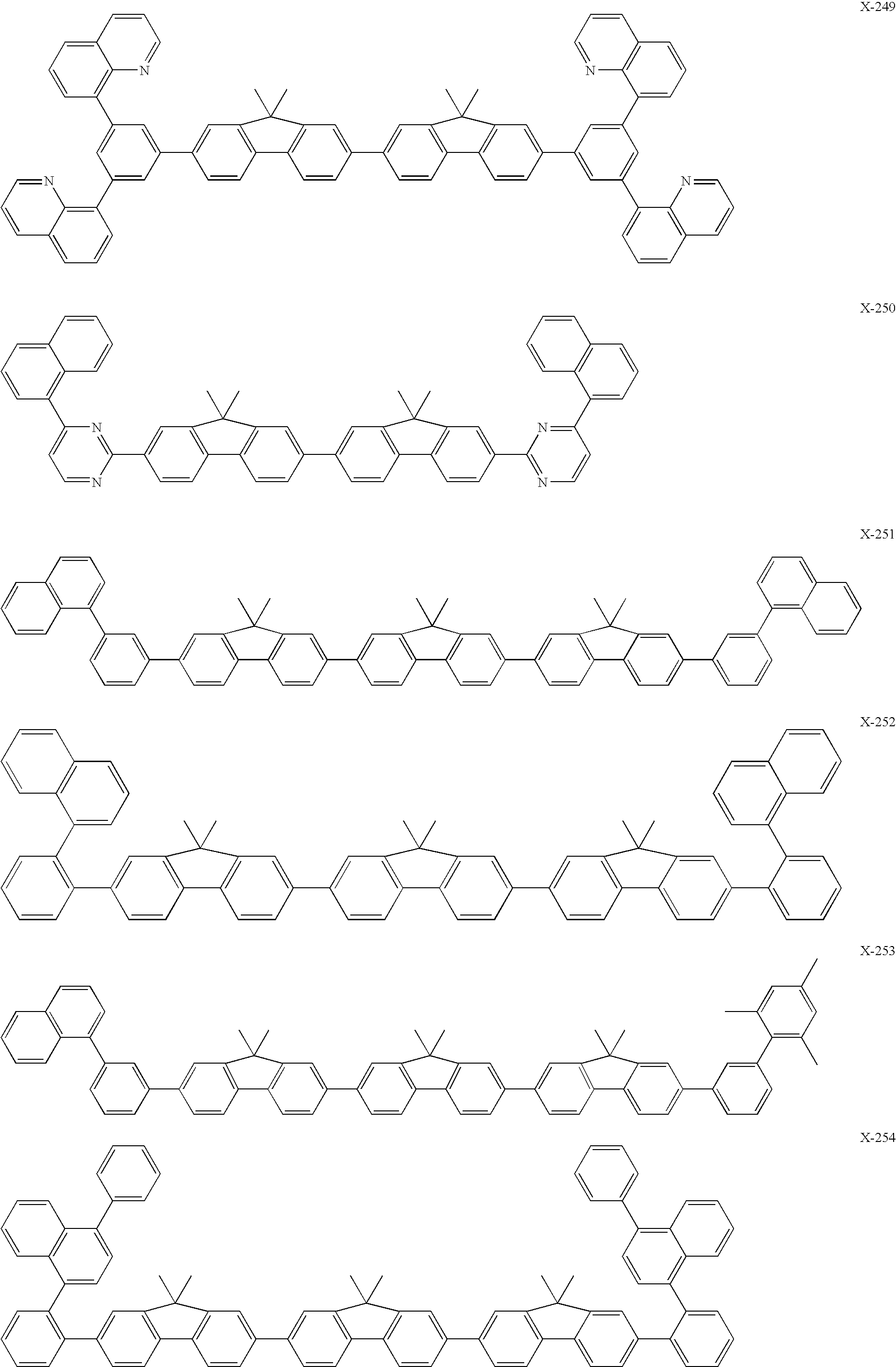

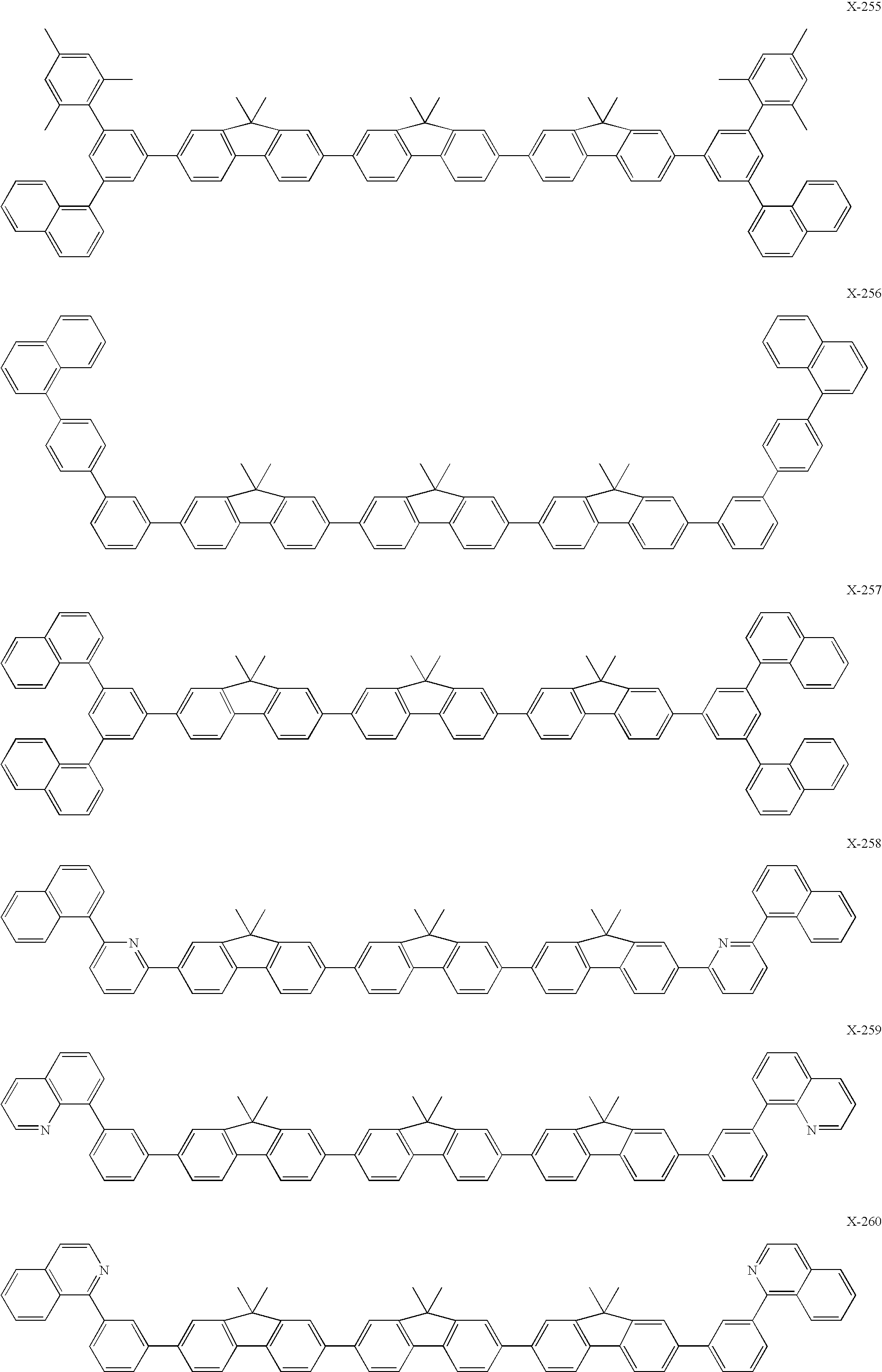

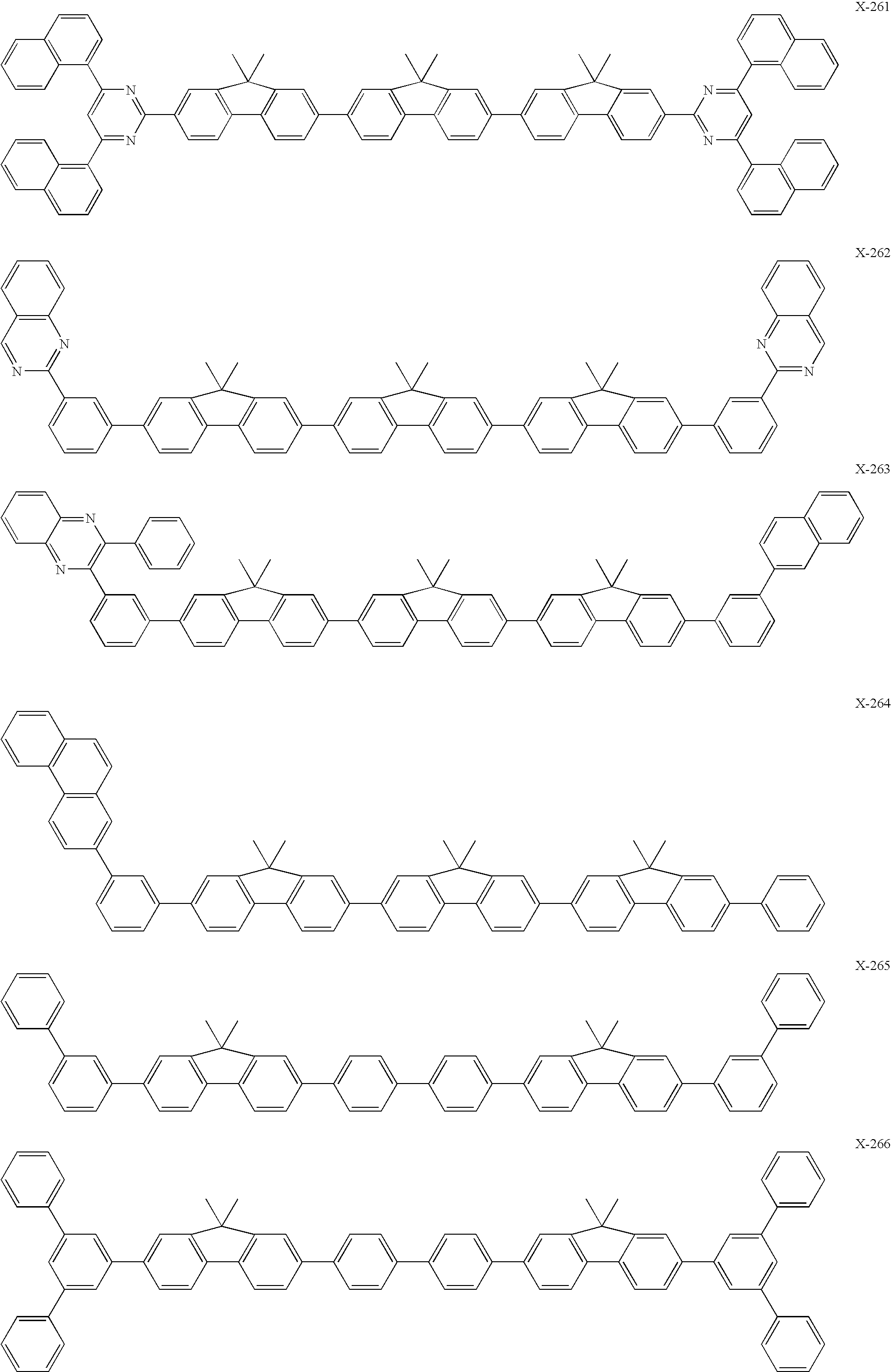

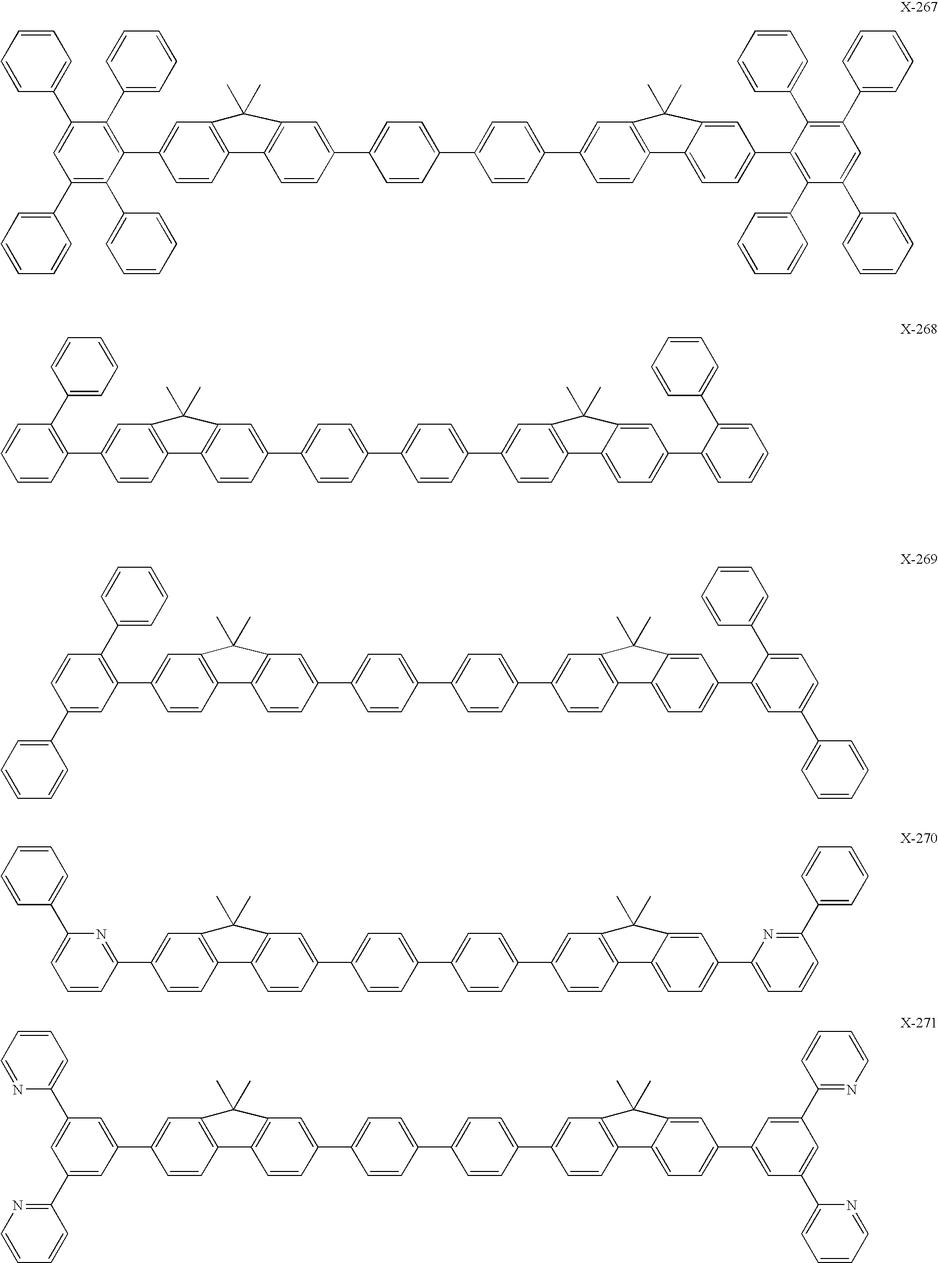

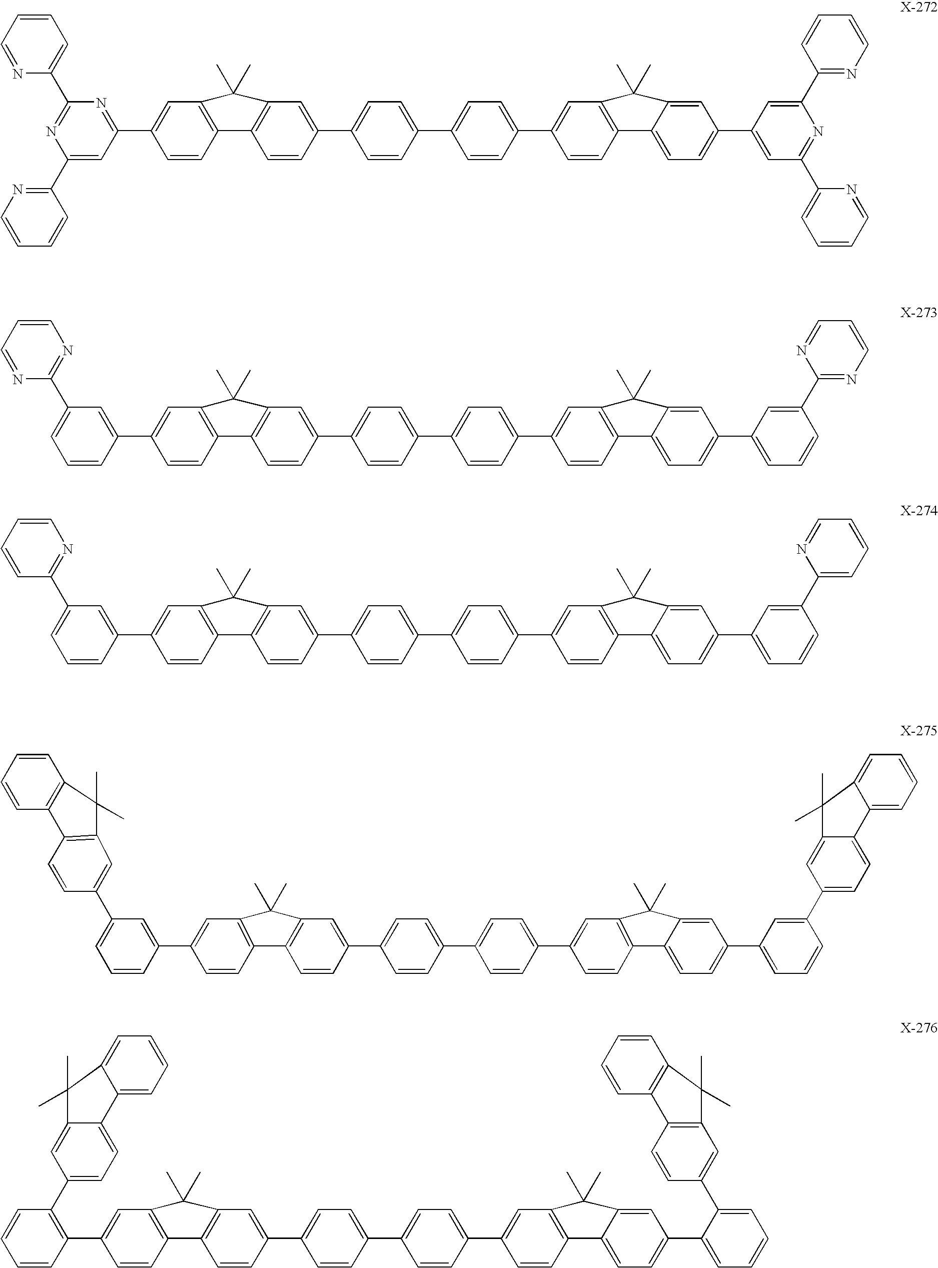

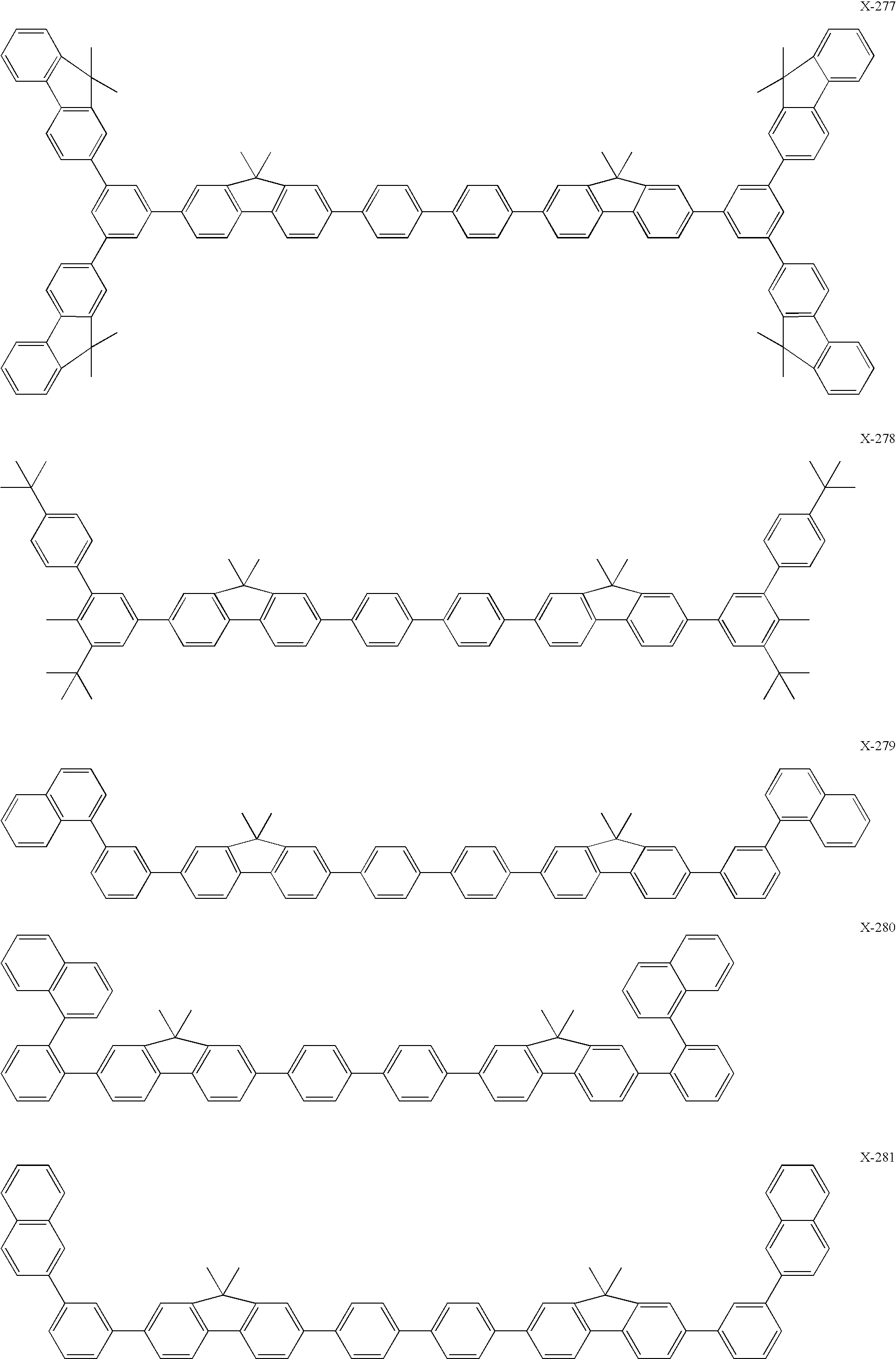

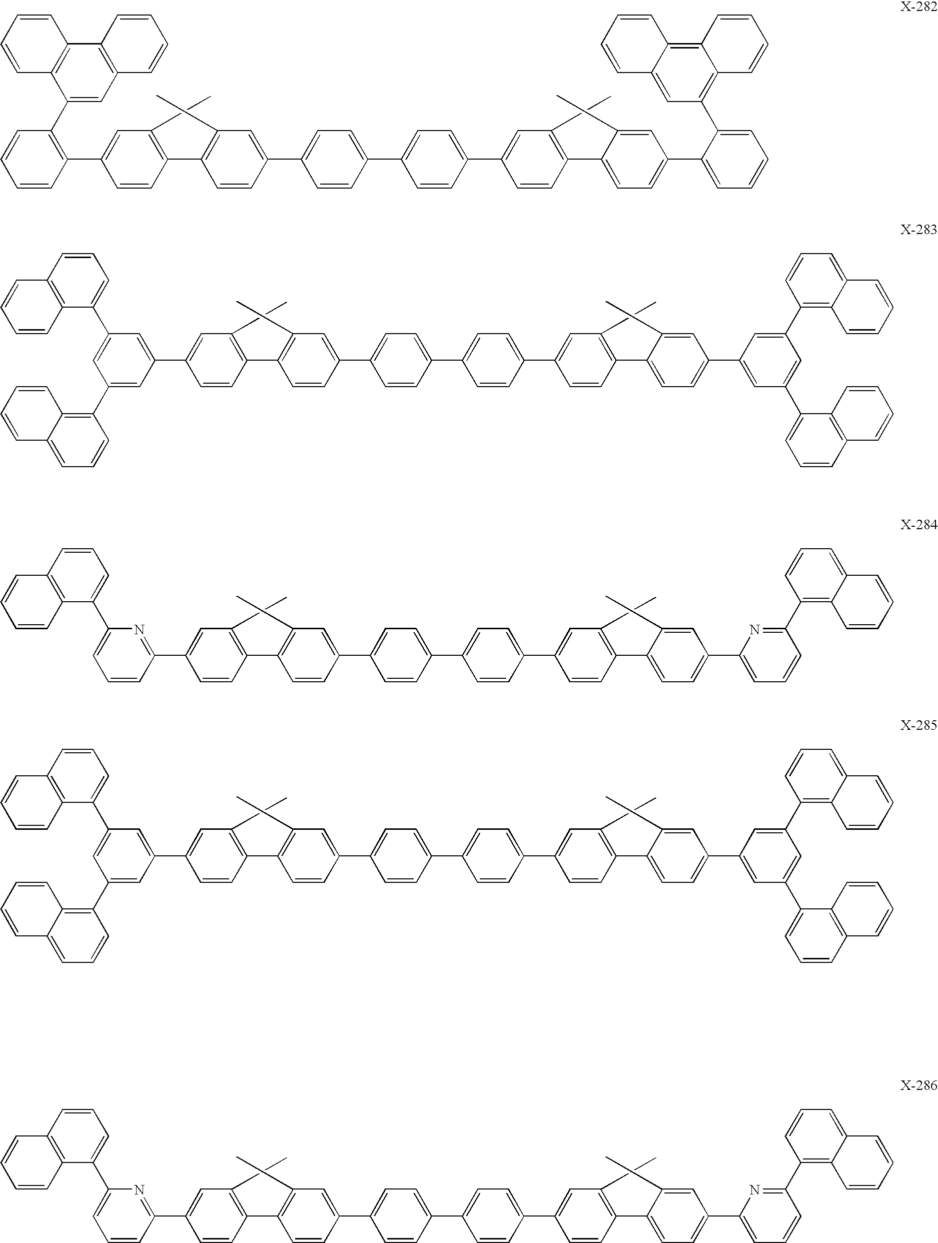

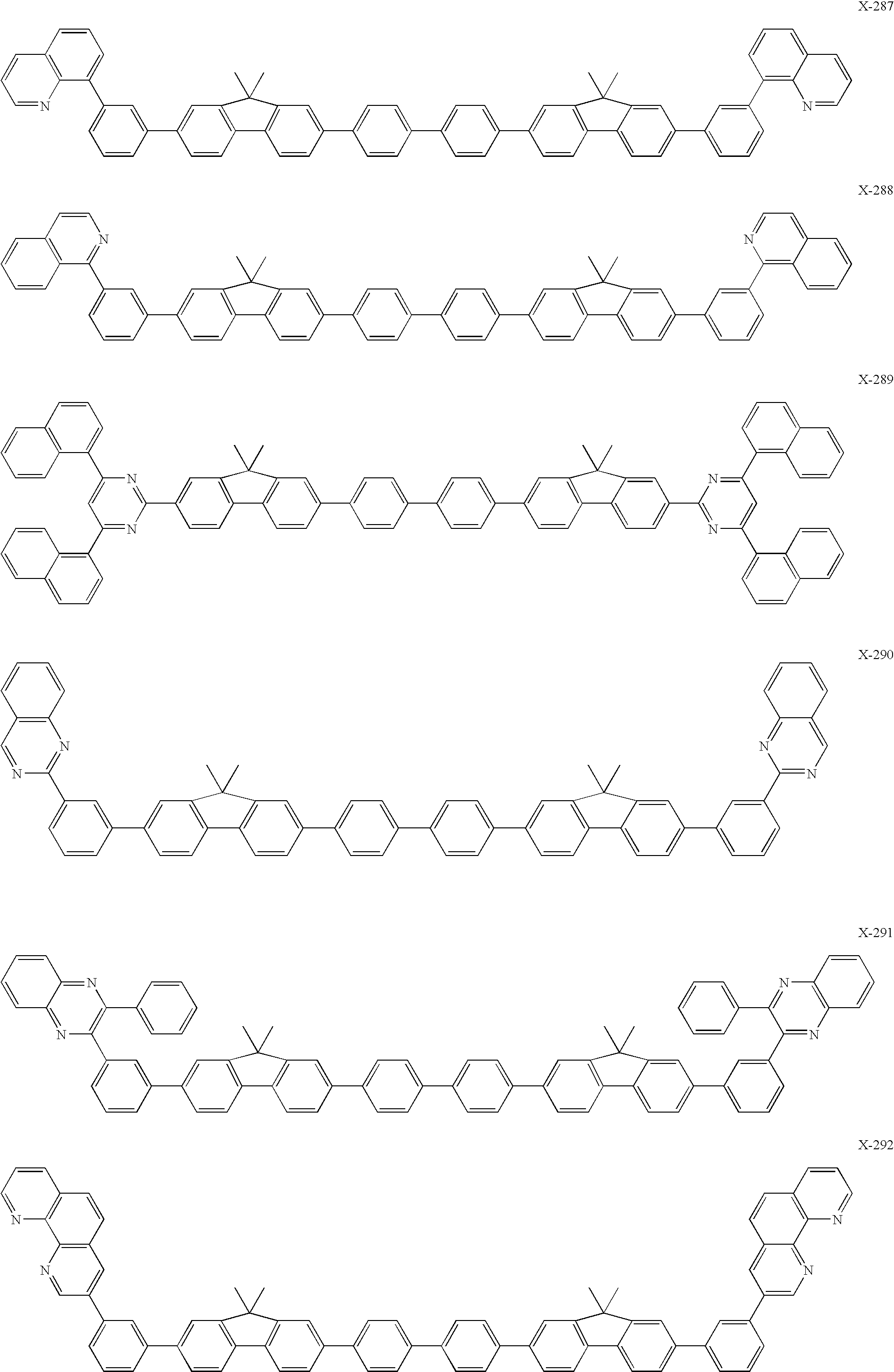

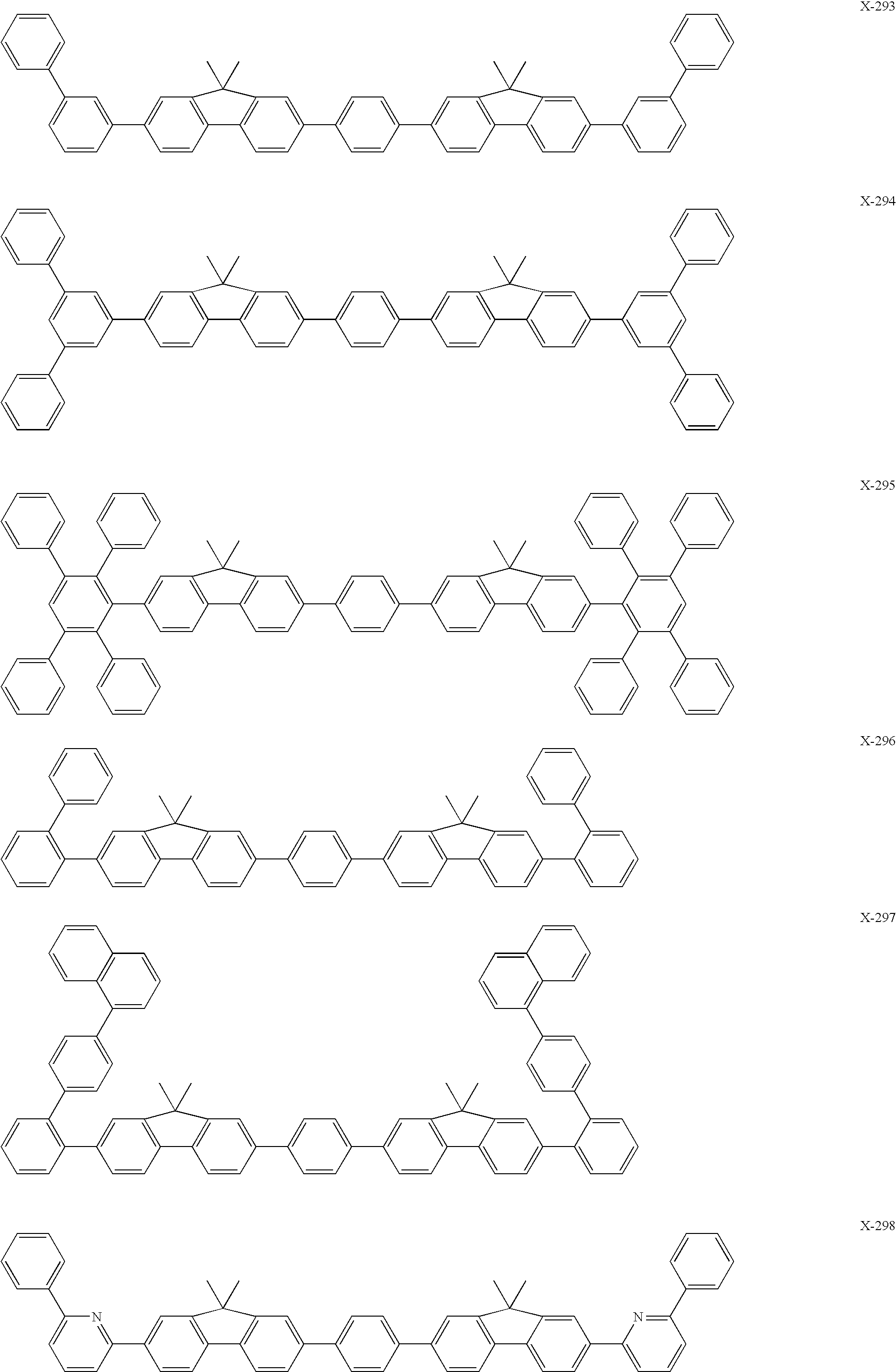

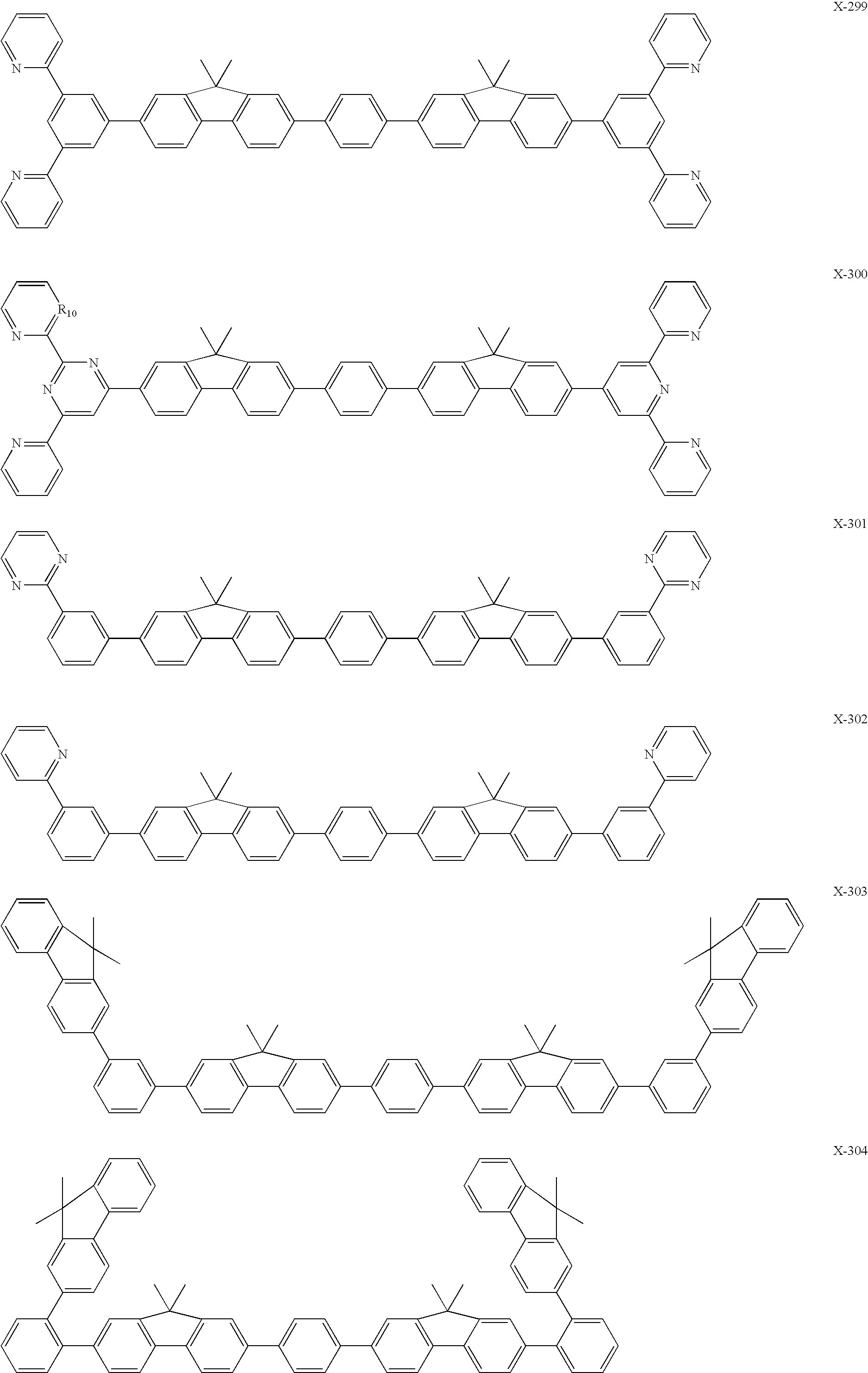

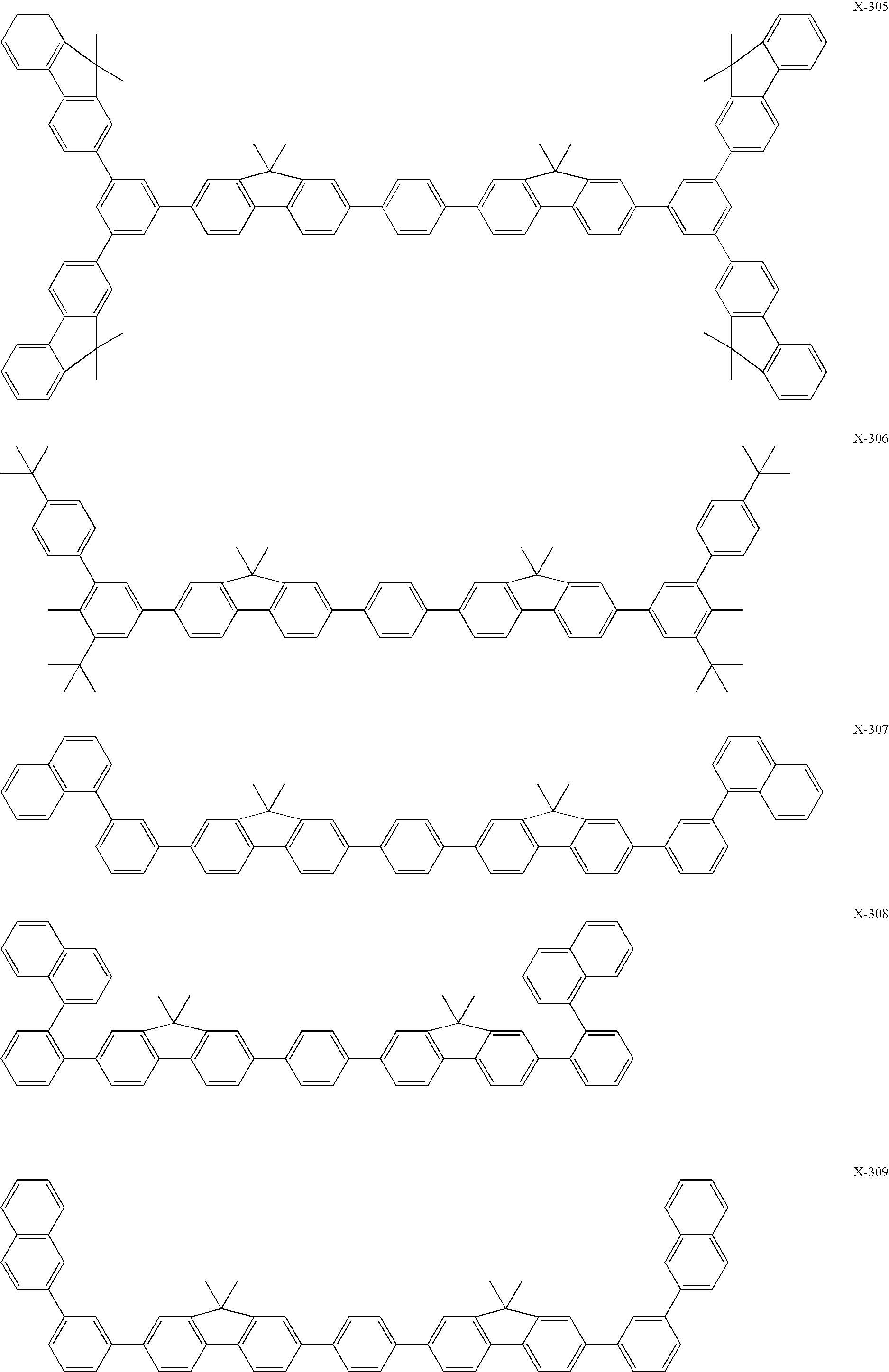

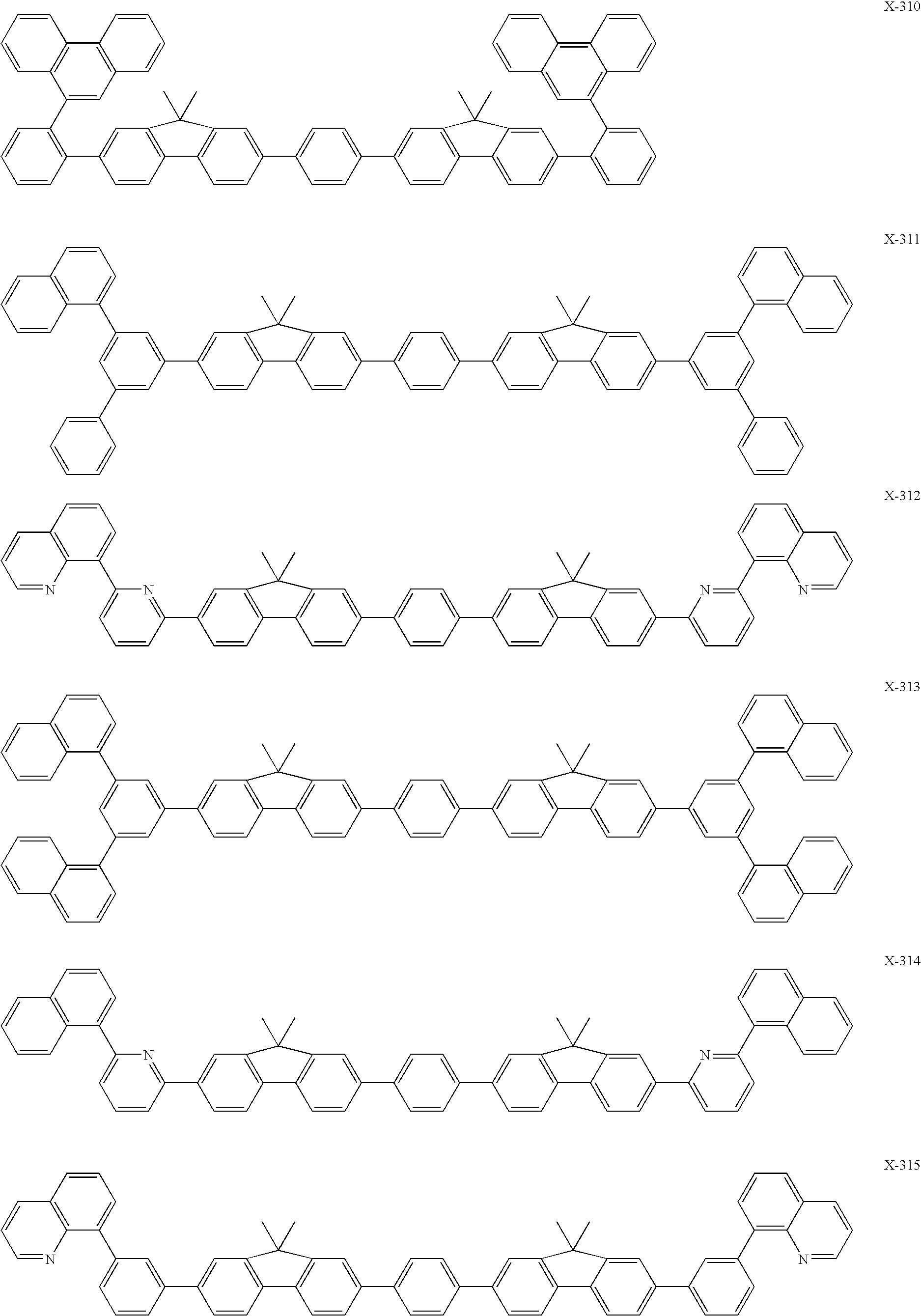

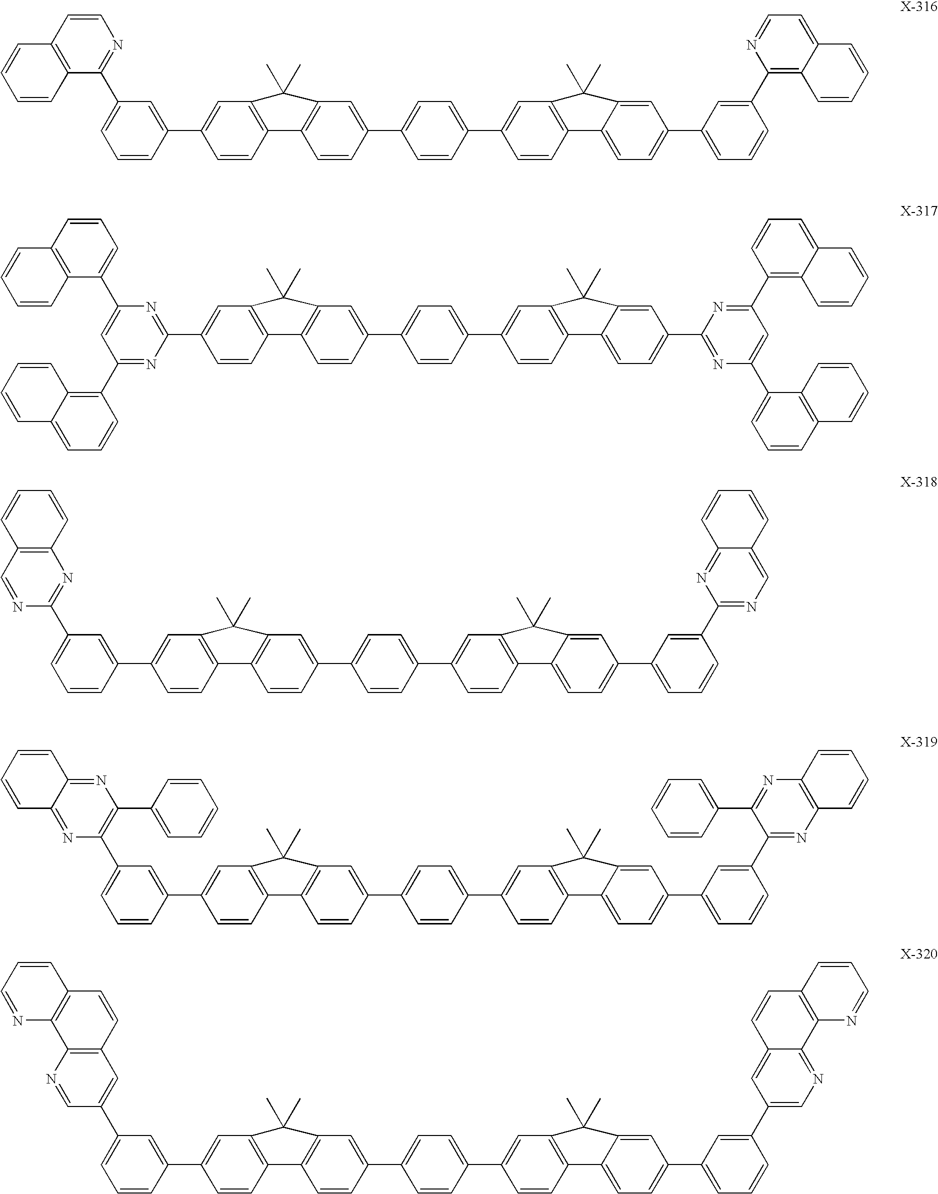

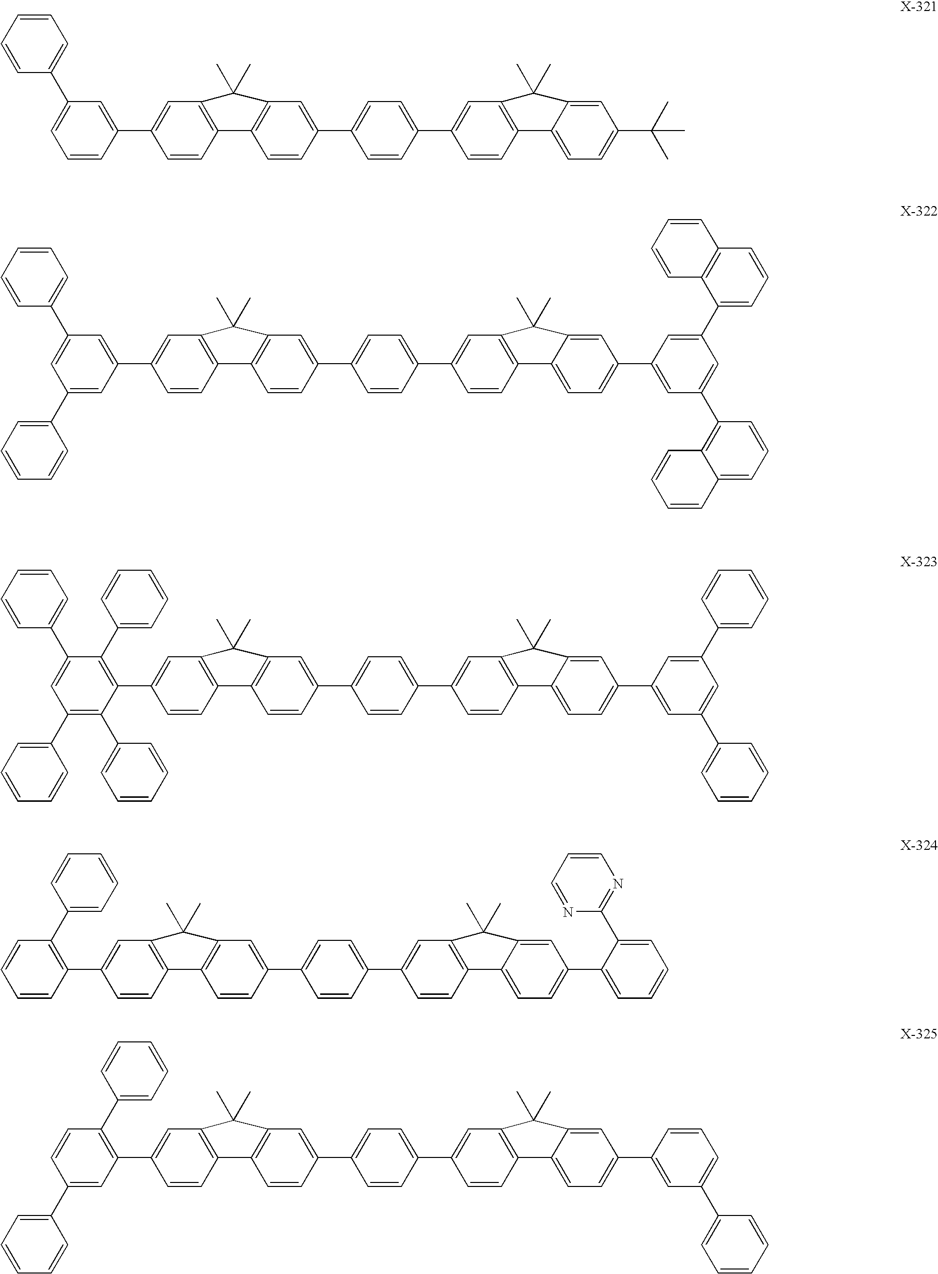

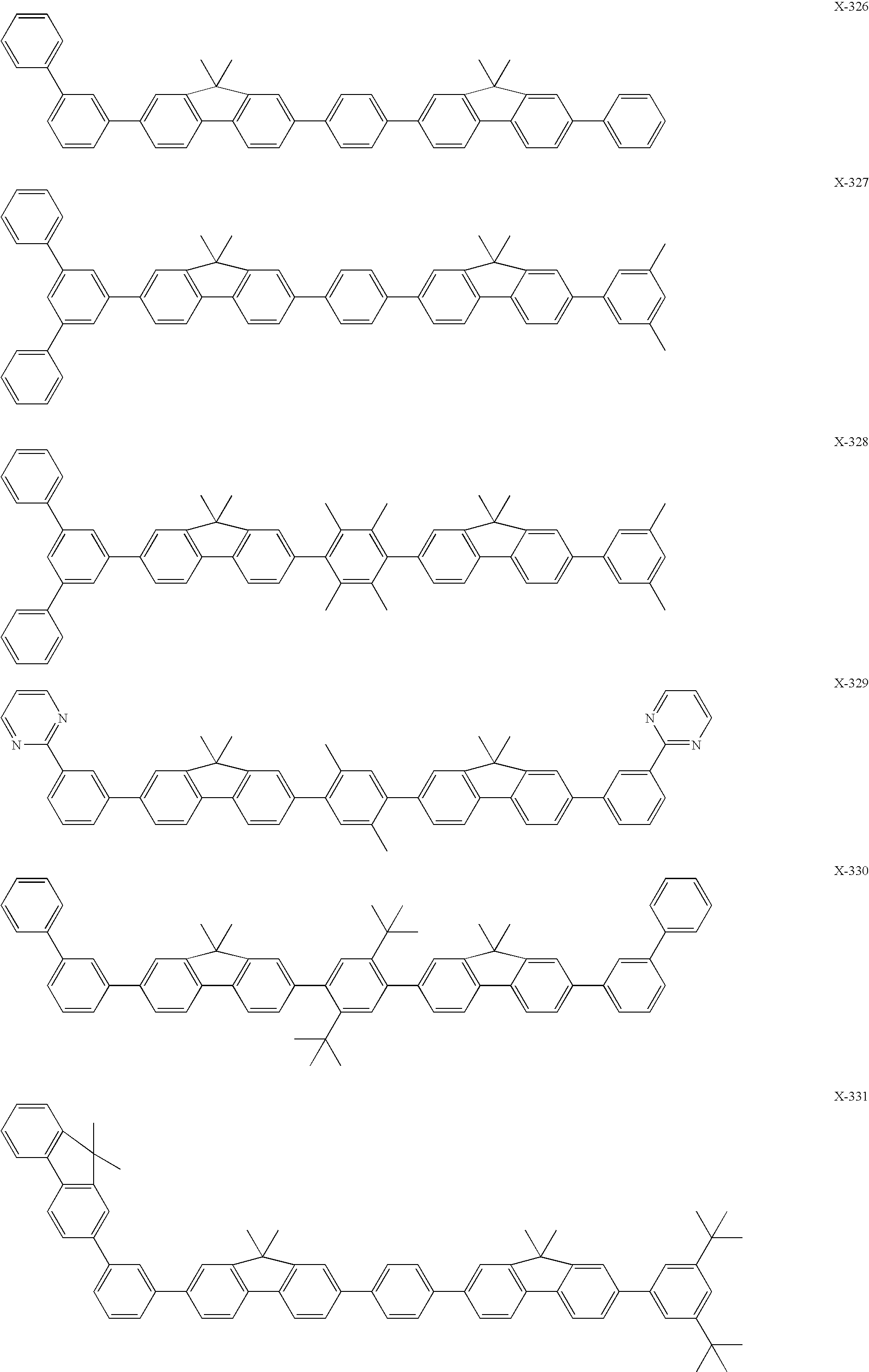

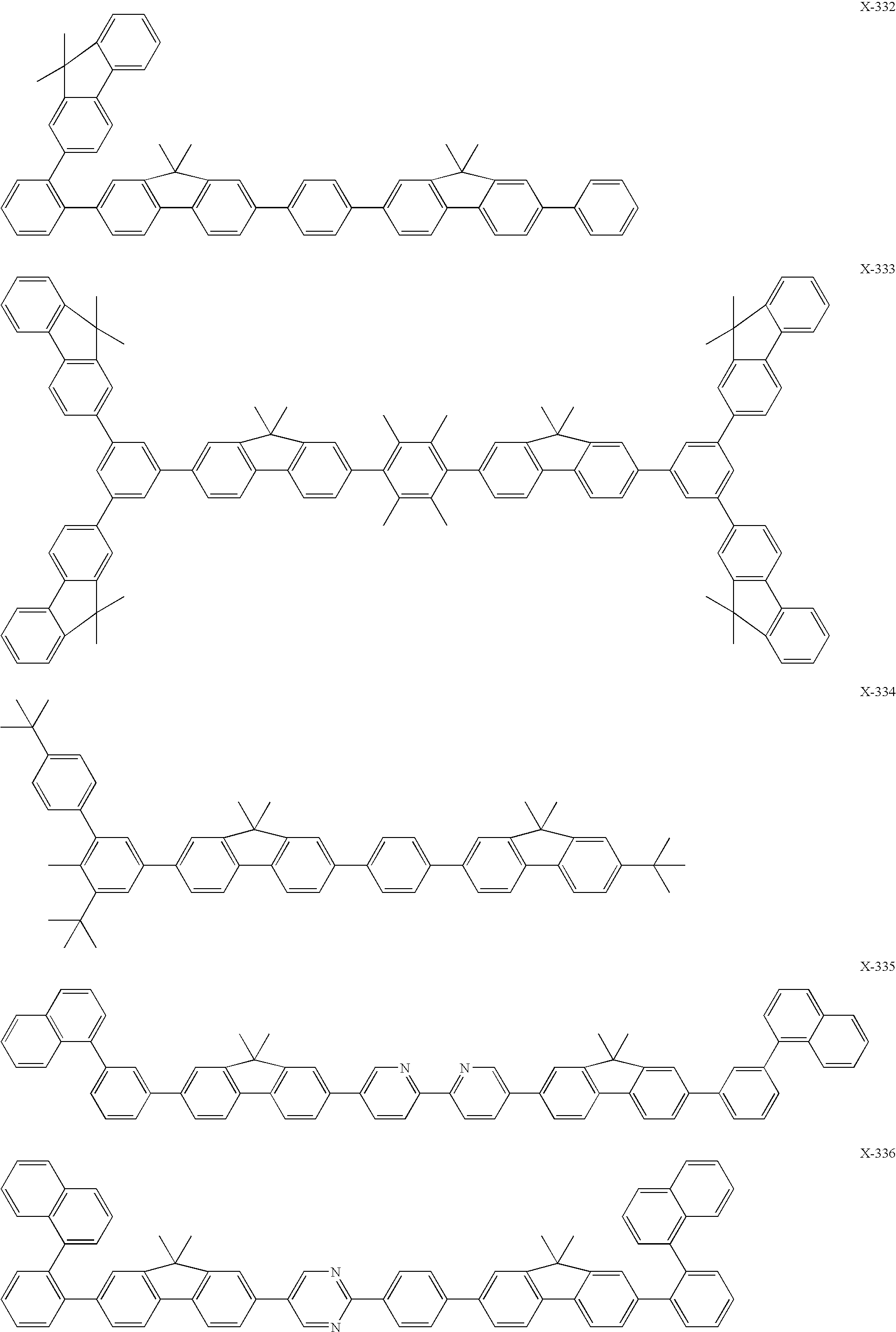

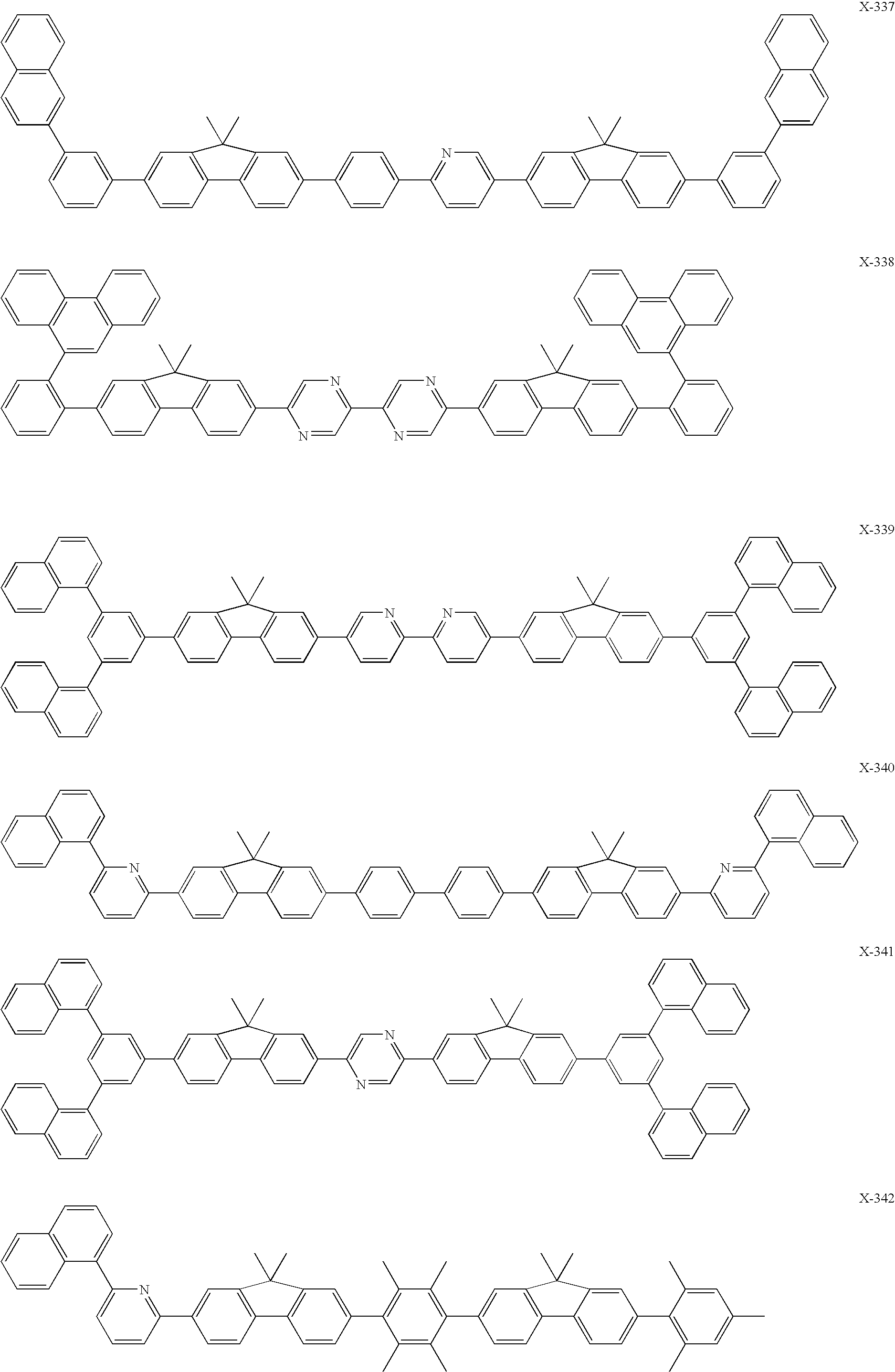

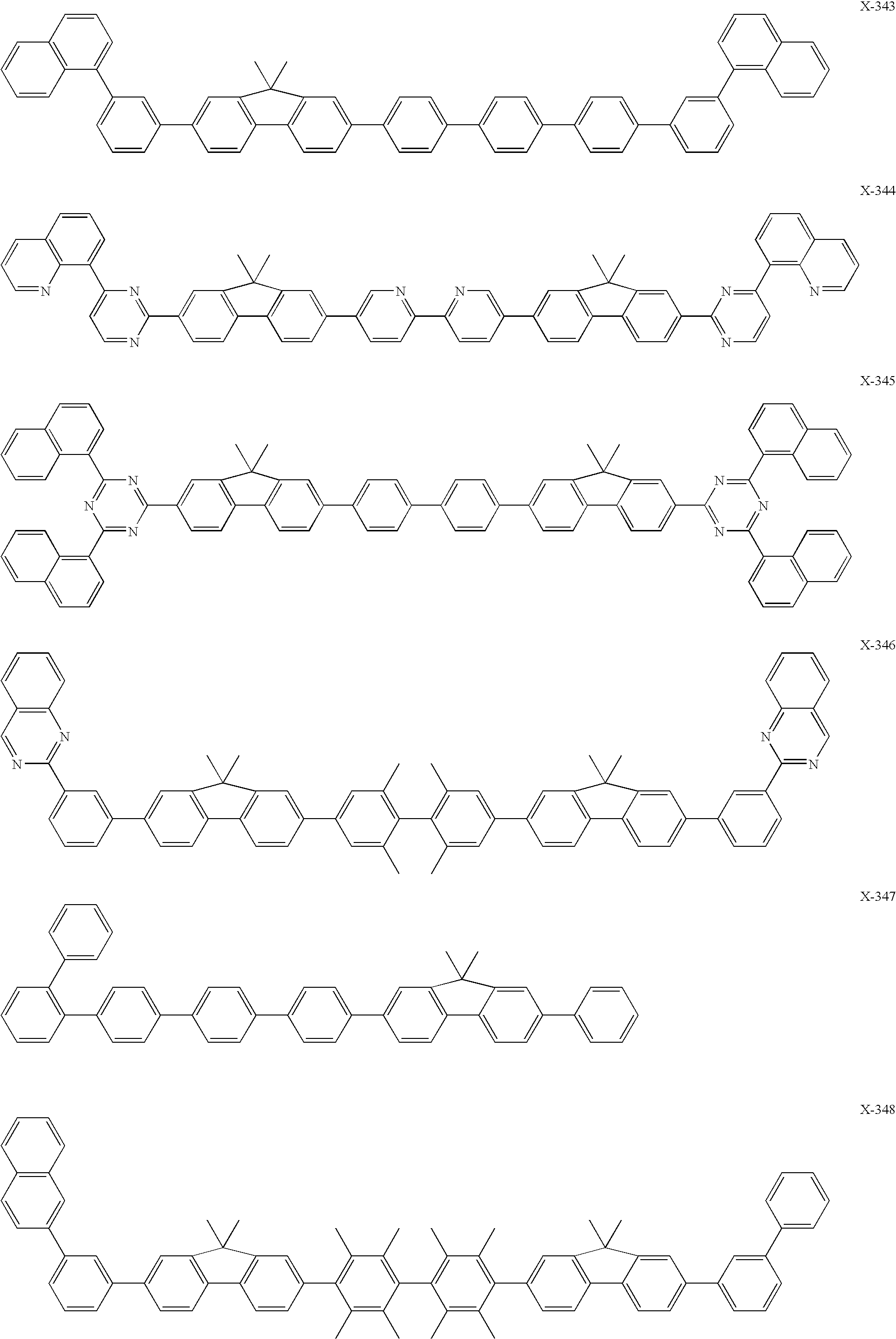

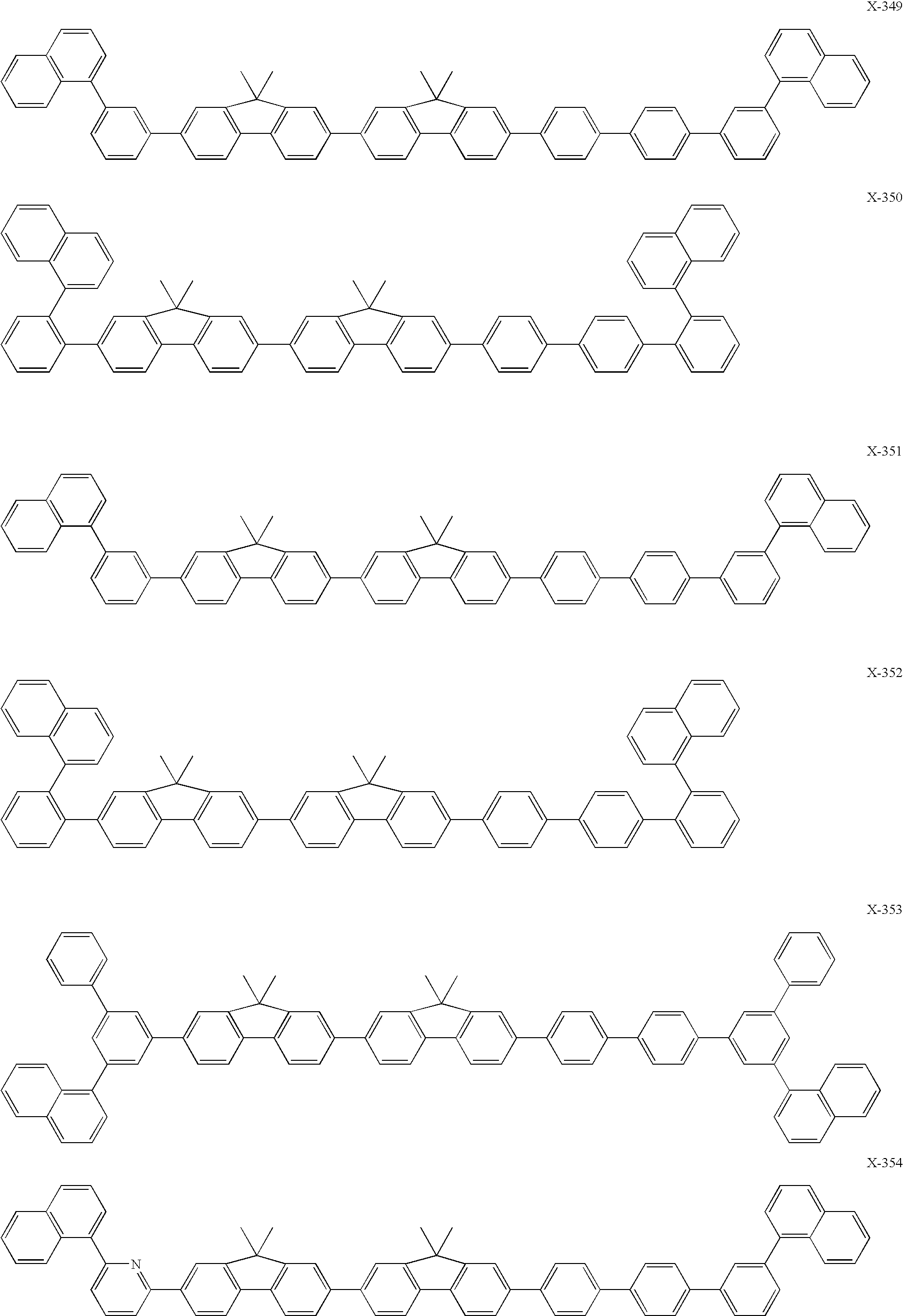

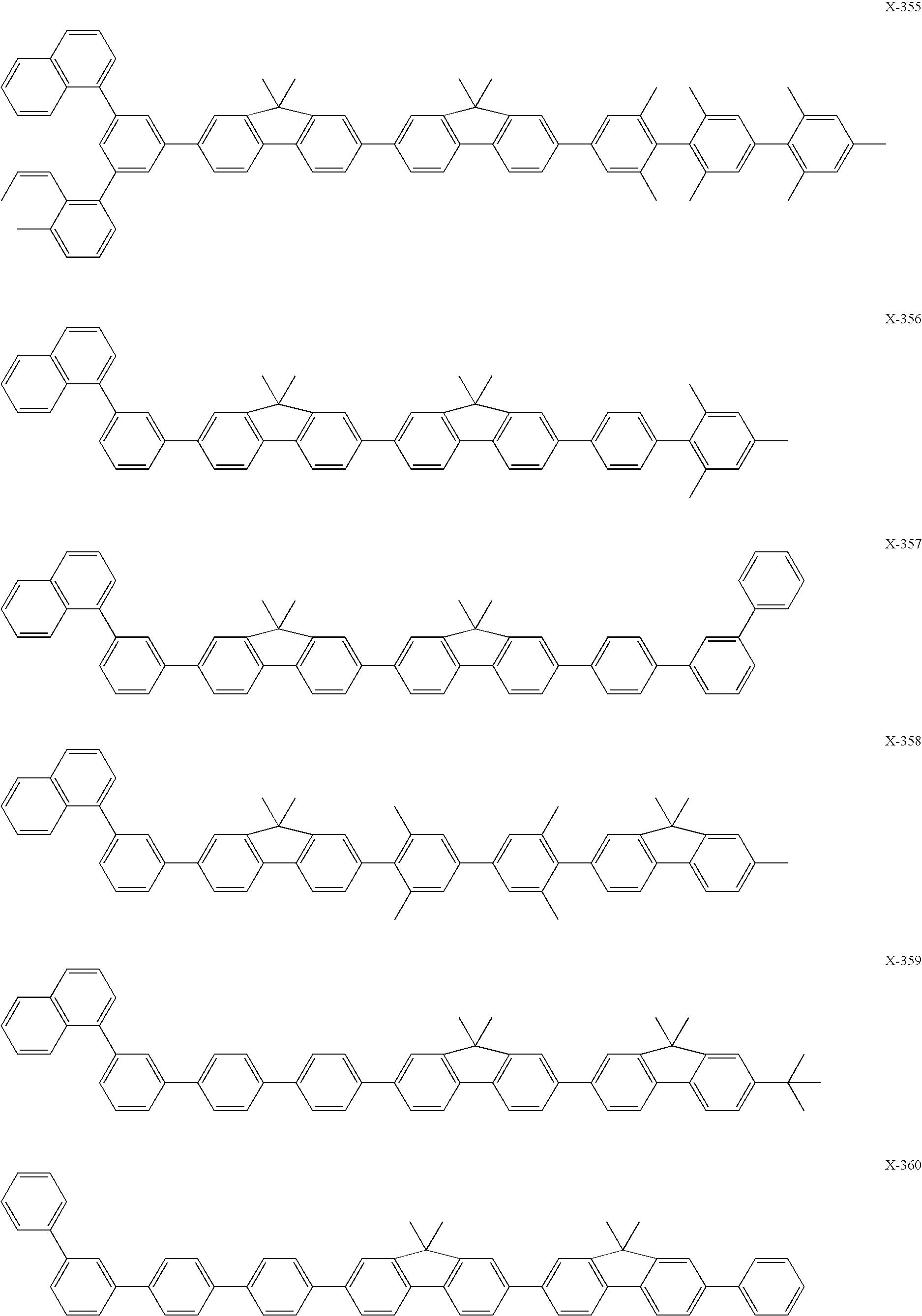

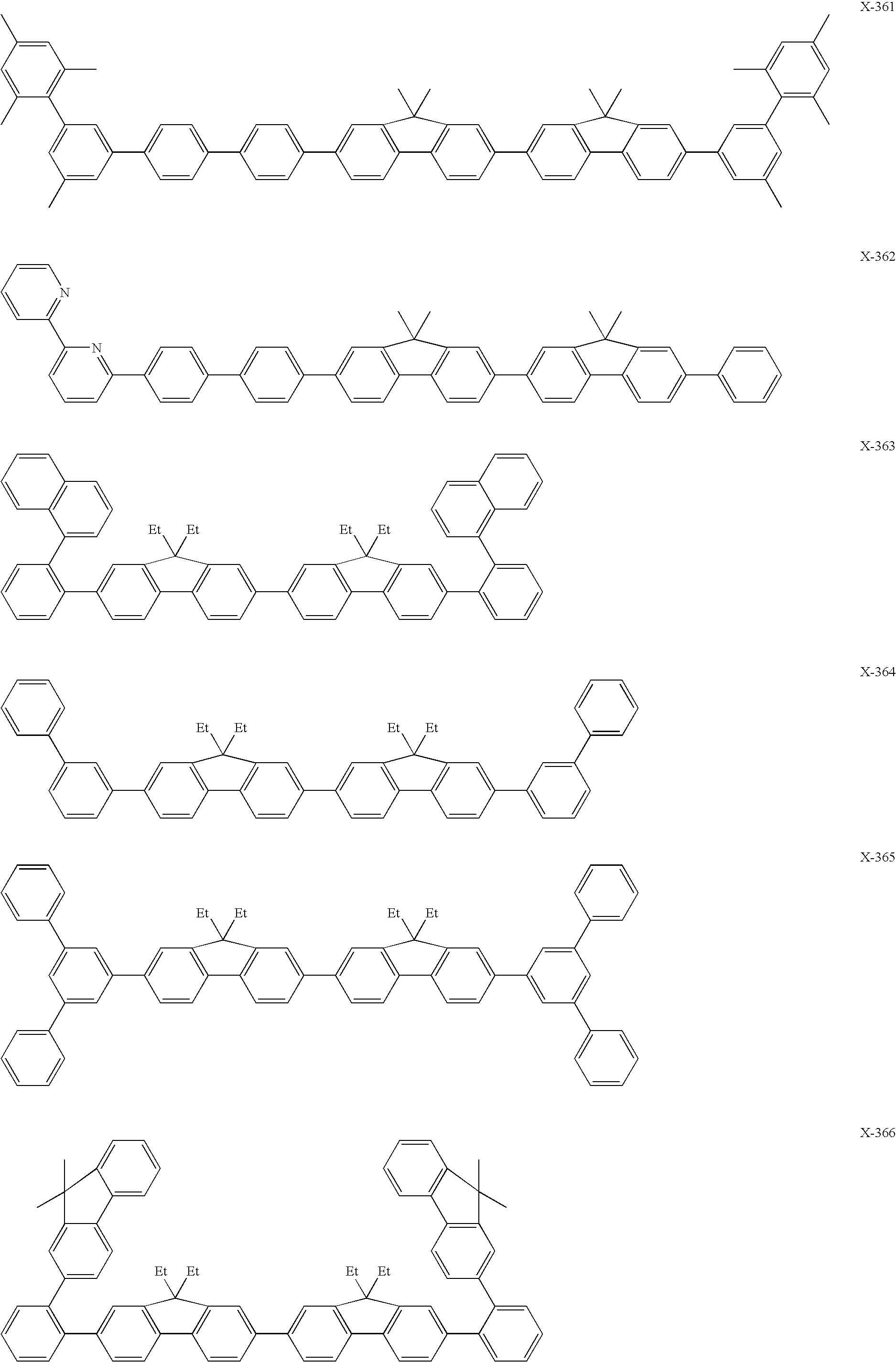

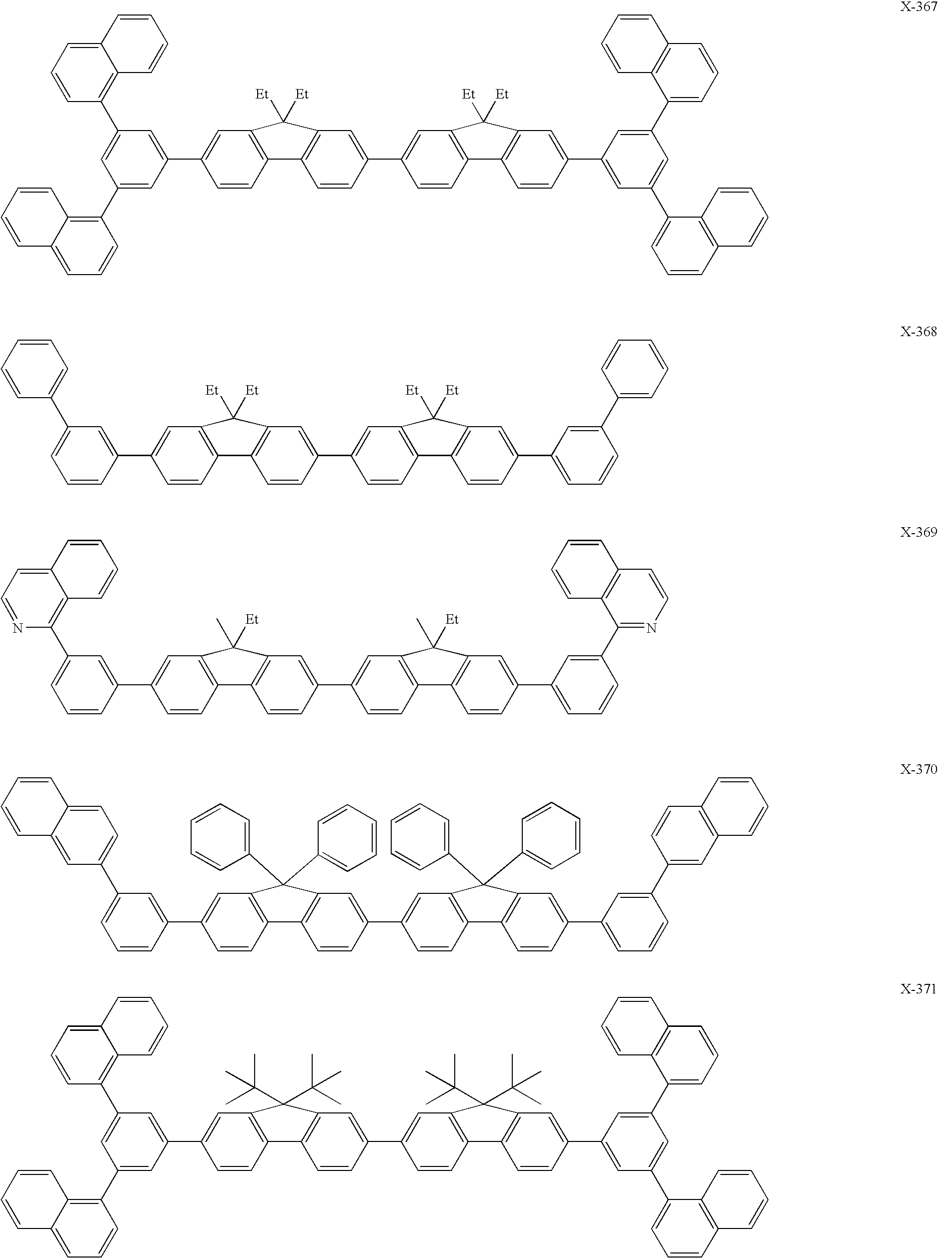

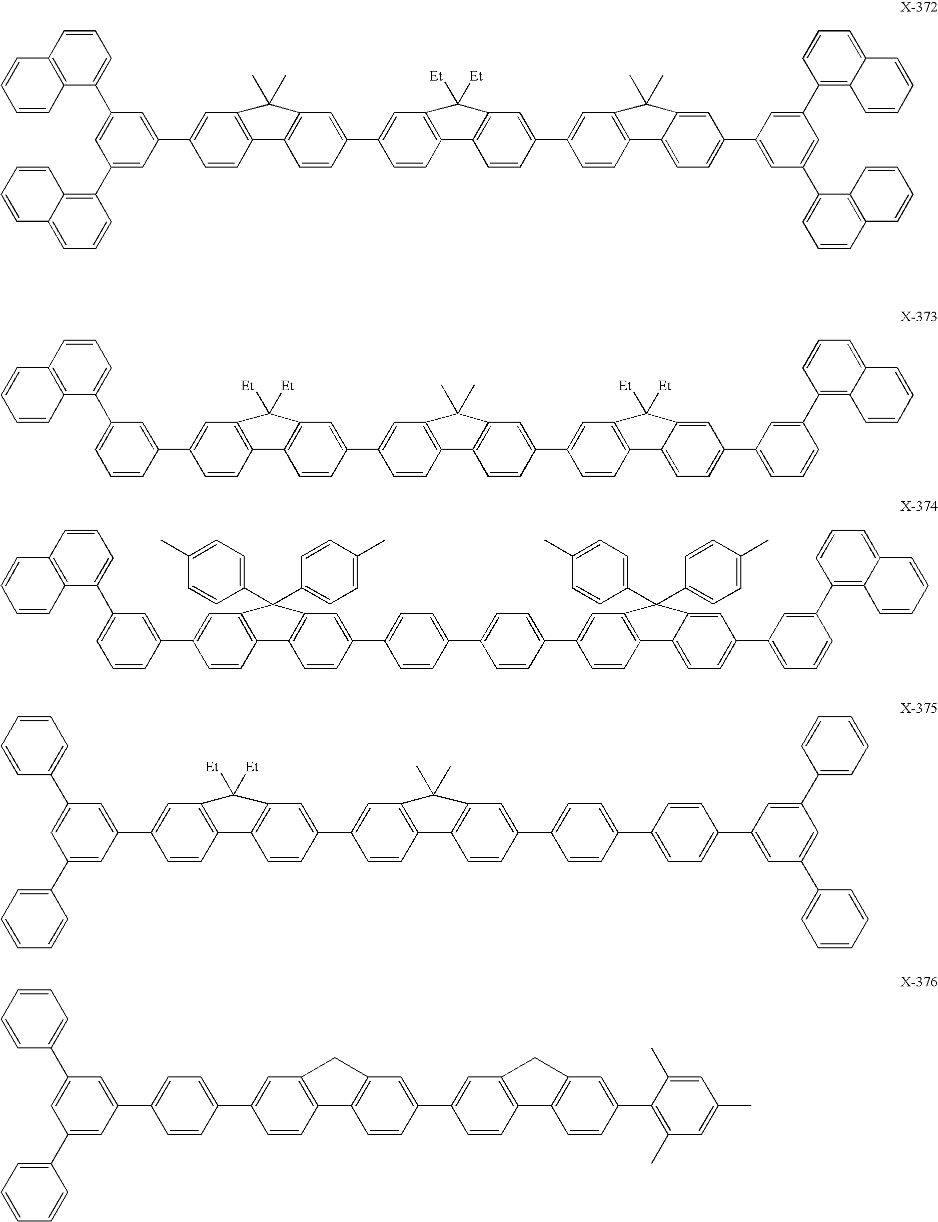

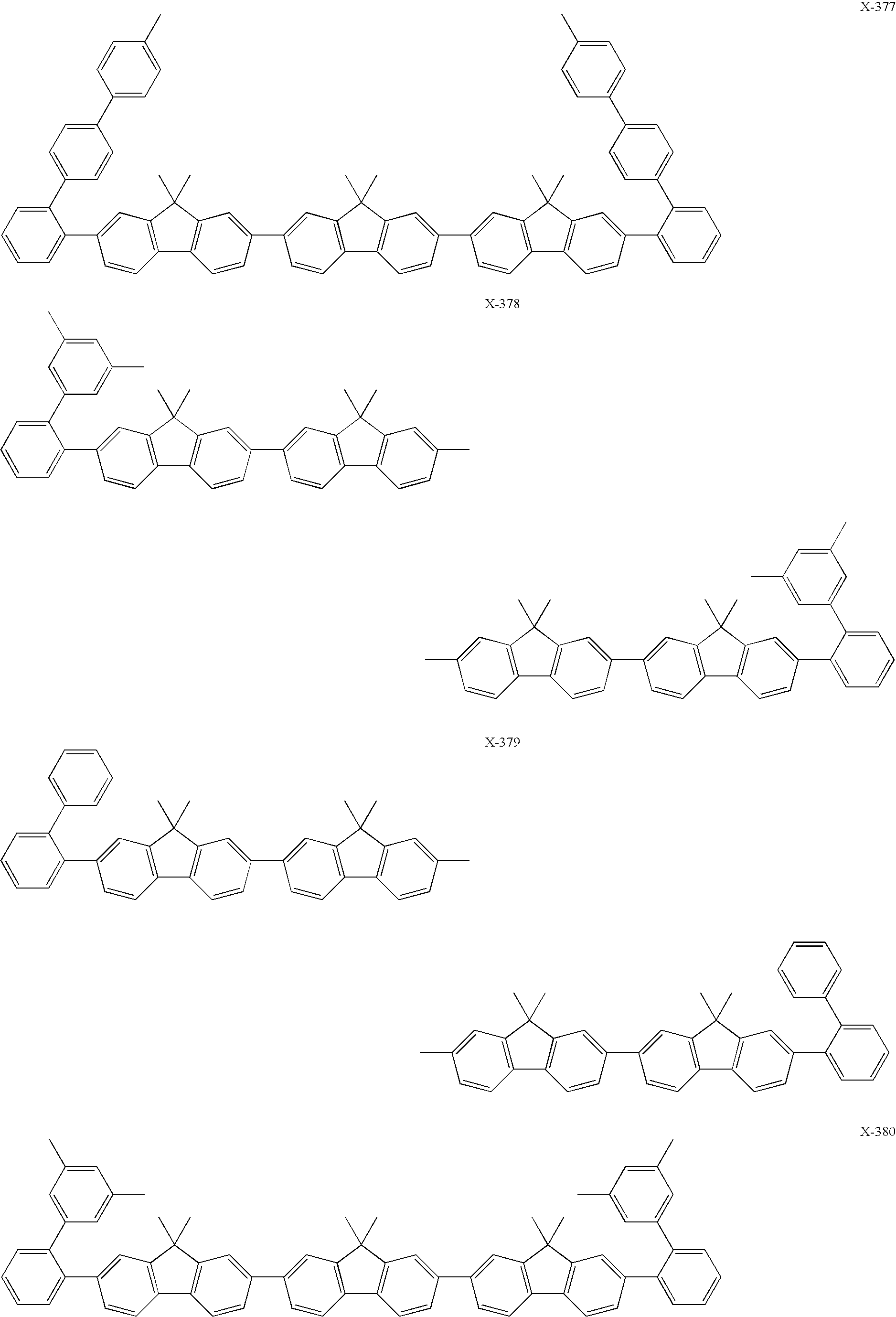

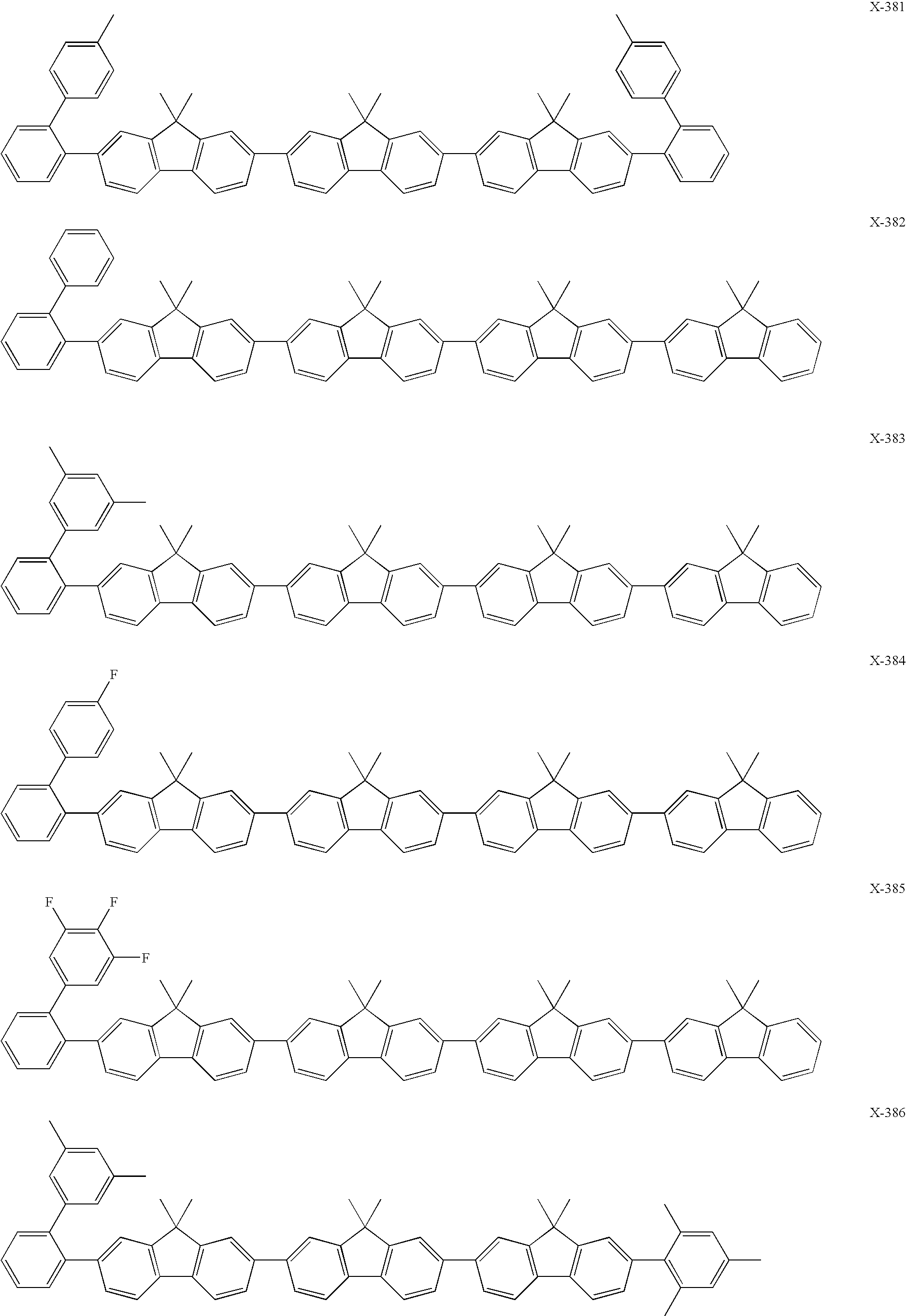

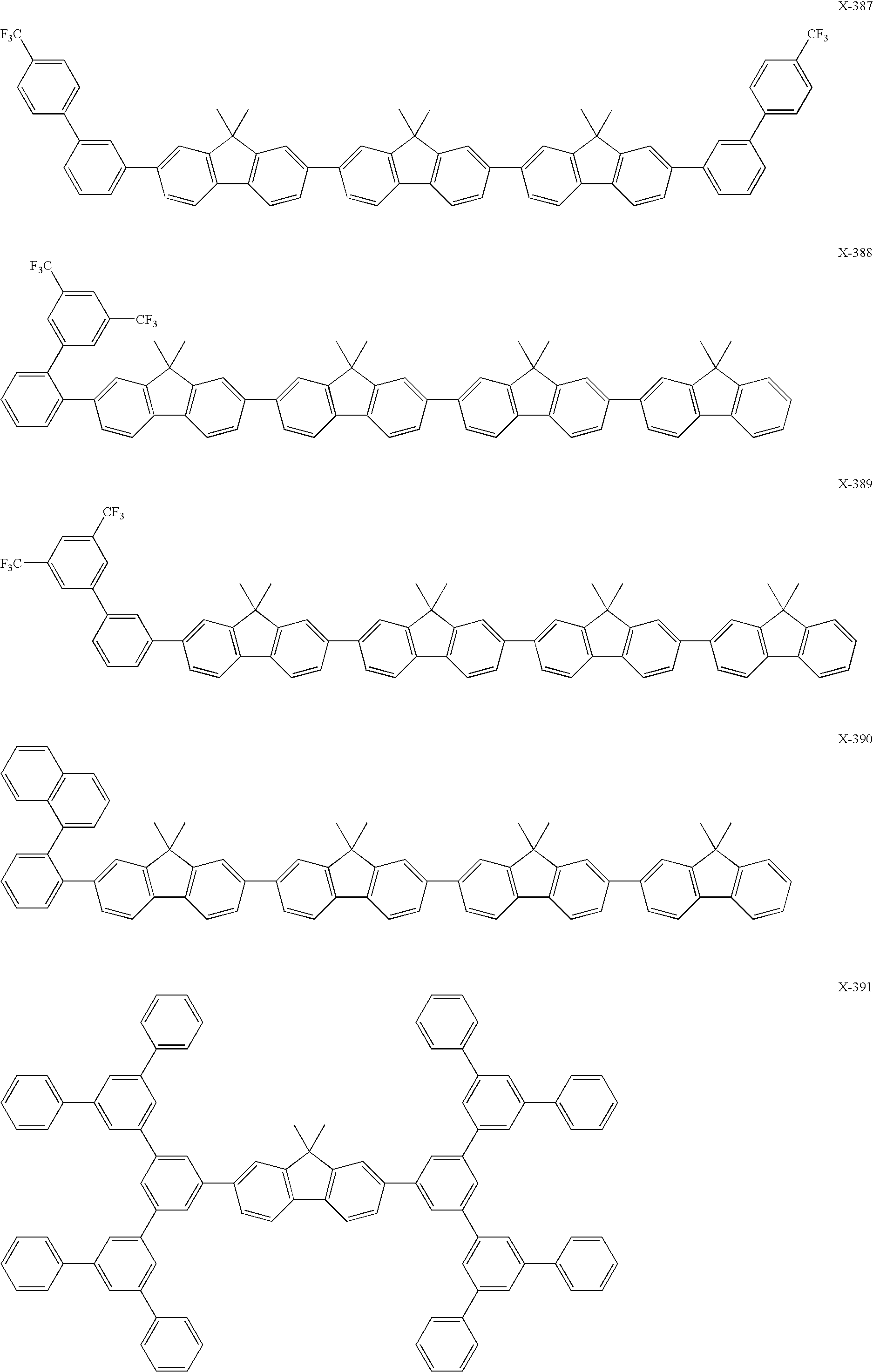

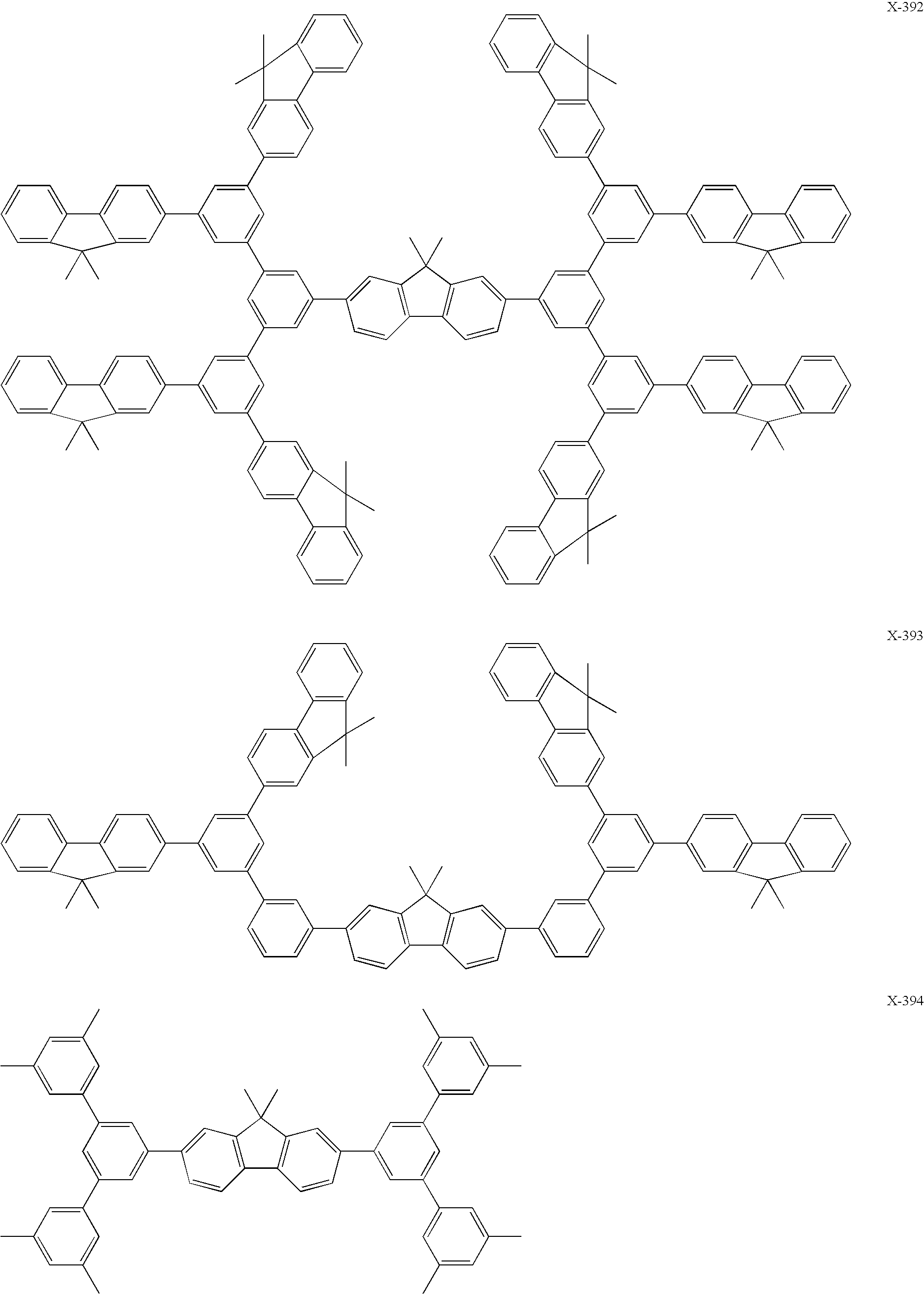

Next, specific structural formulae of a guest compound will be representatively shown.

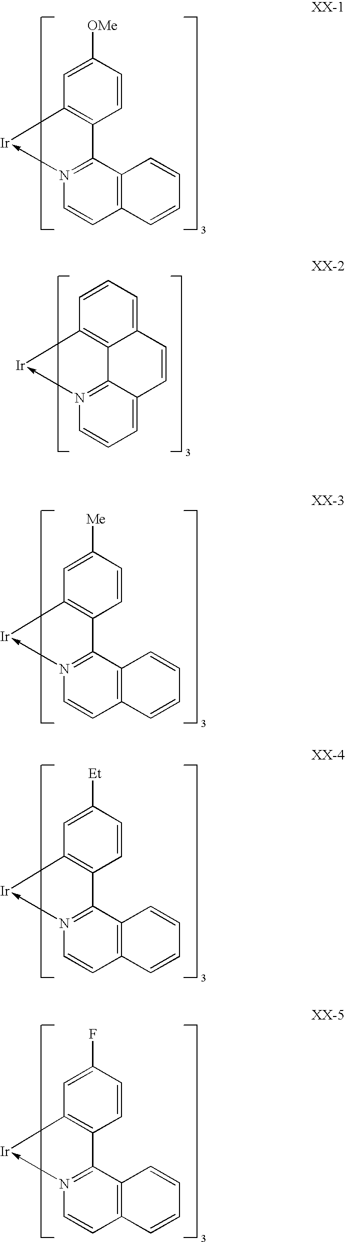

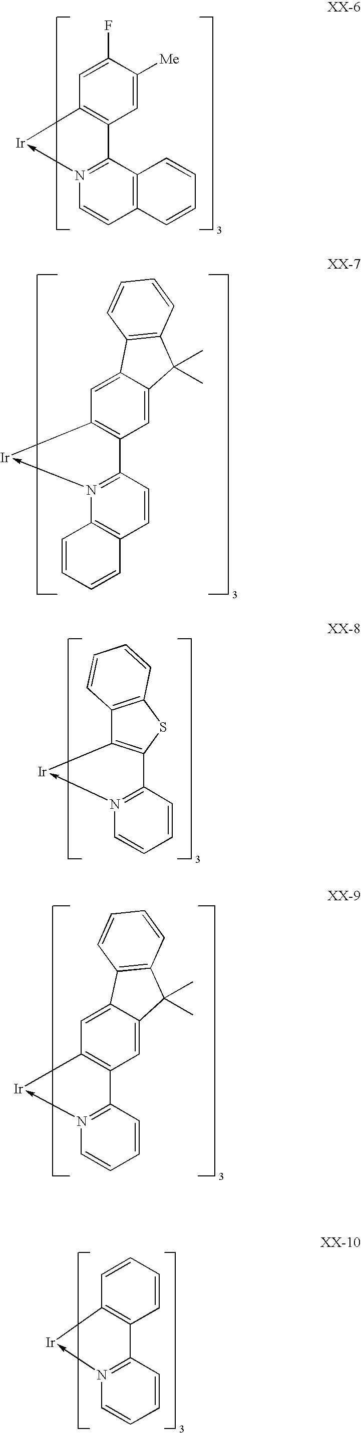

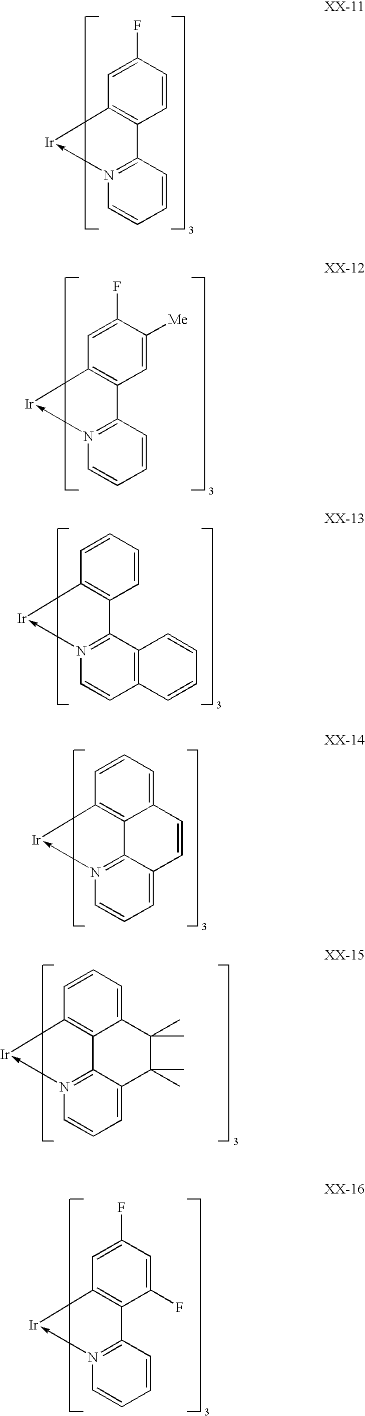

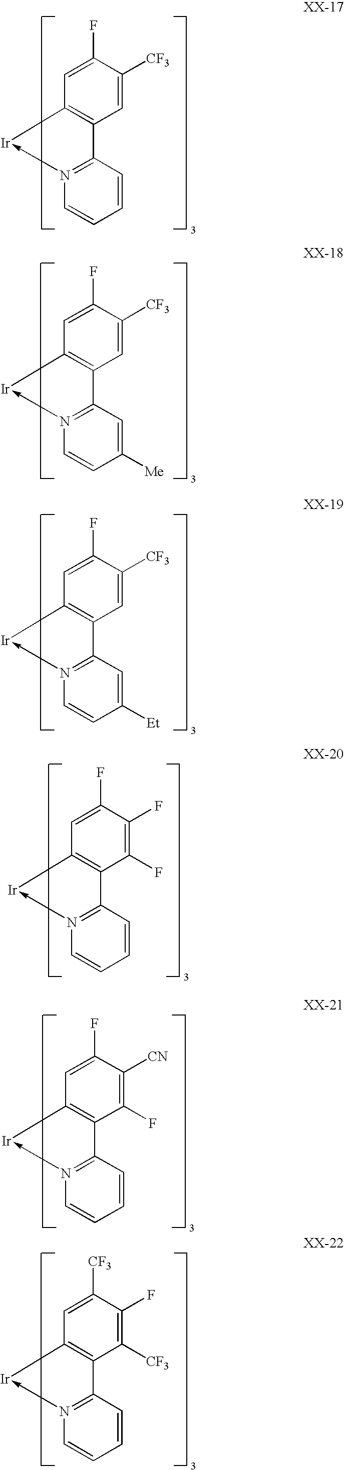

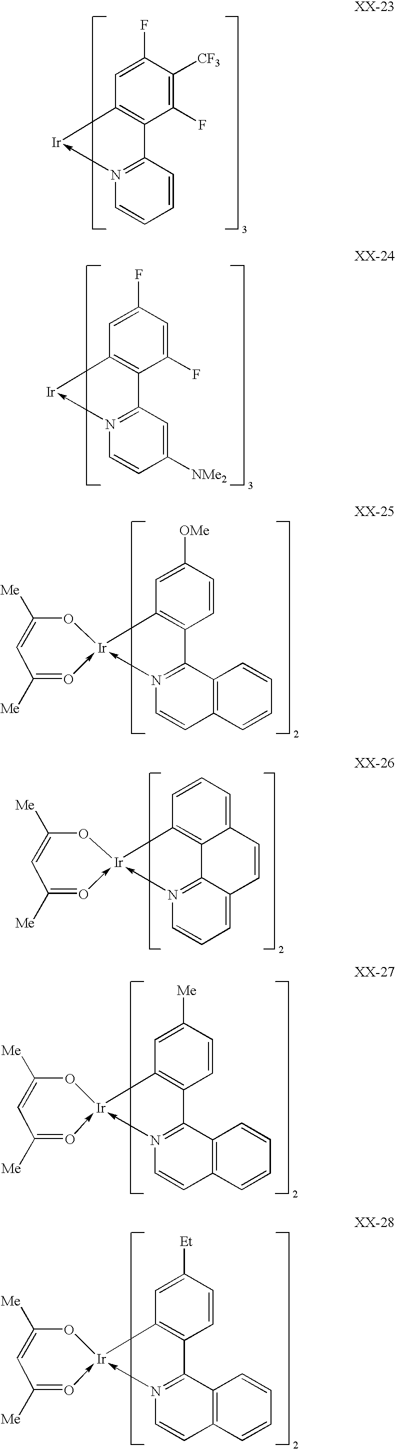

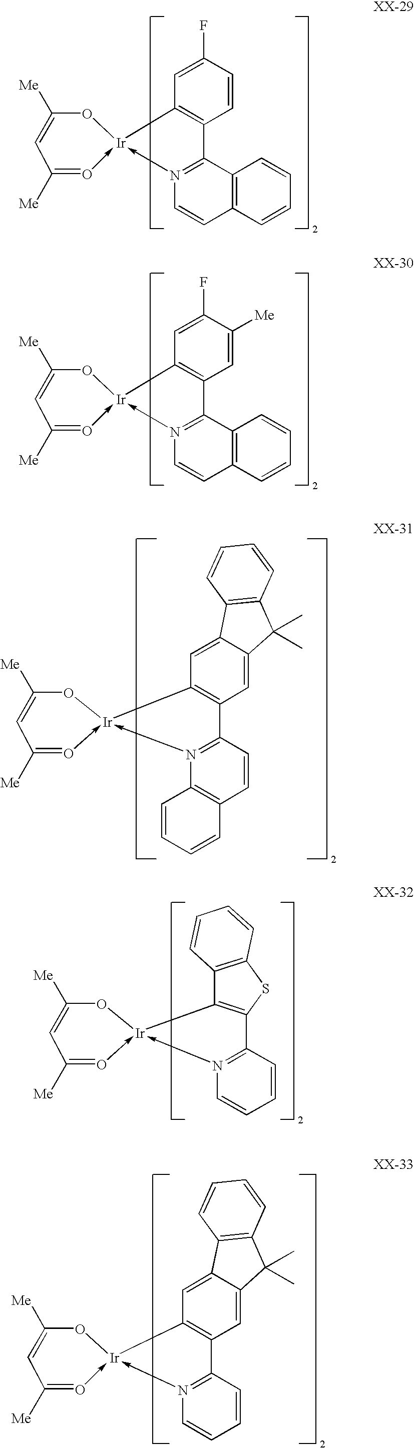

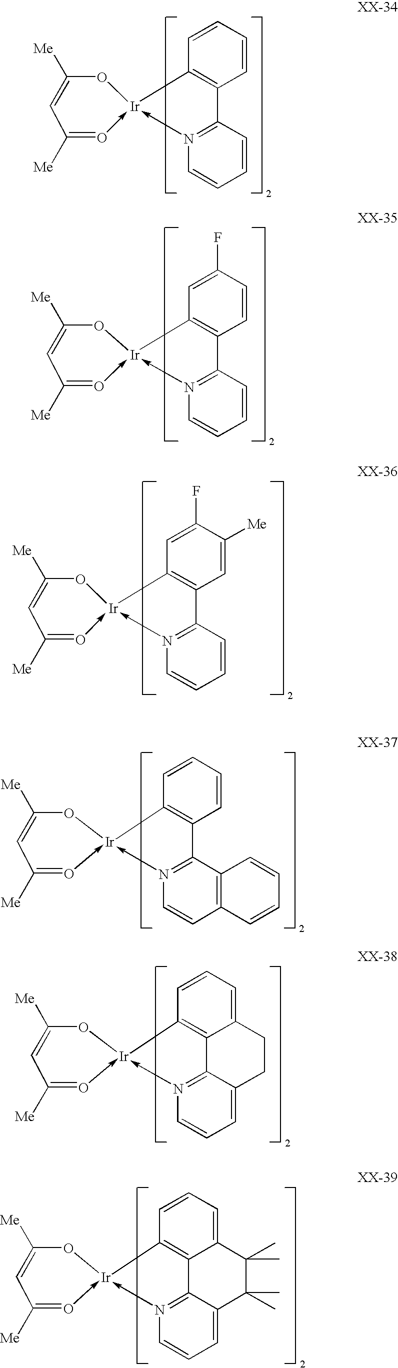

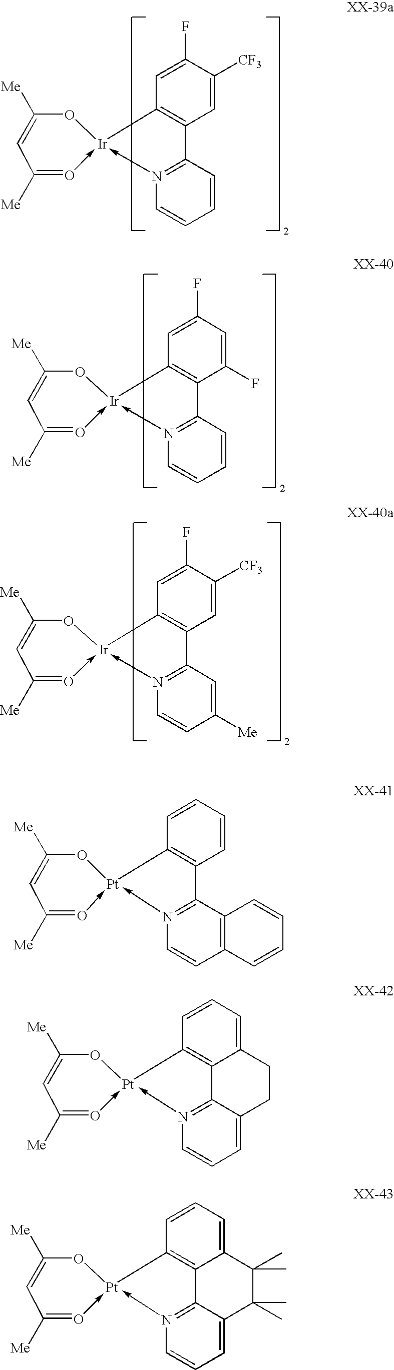

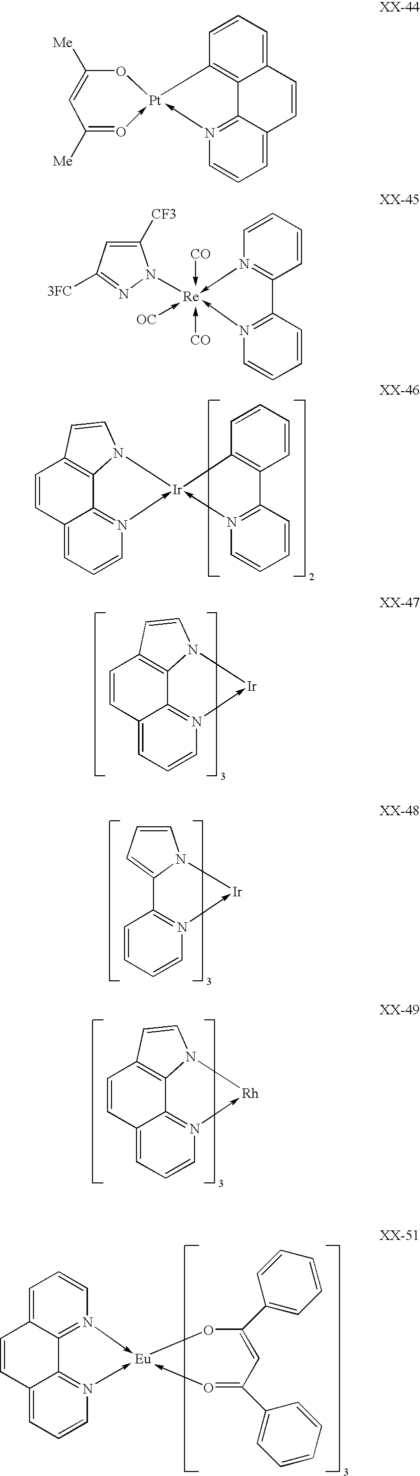

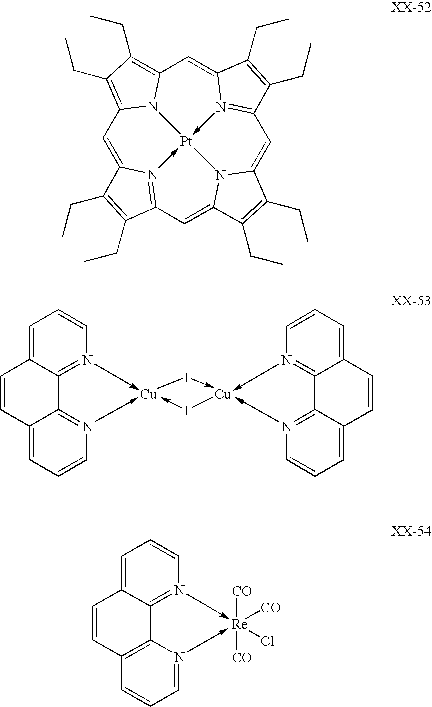

Next, the organic electroluminescent device in accordance with the present invention will be described.

The organic electroluminescent device of the present invention comprises a pair of electrodes and at least one layer comprising an organic compound sandwiched between the electrodes, and at least one of the at least one layer comprising the organic compound, preferably a light-emitting layer comprises at least one kind of the compound of the present invention preferably as a host of the light-emitting layer.

When the compound of the present invention is used for a host of a light-emitting layer, there may be used, as a guest molecule, any generally known fluorescent material and phosphorescent material, with the phosphorescent material being preferred. In order to obtain a light-emitting device having a high efficiency, it is preferable to use a metal coordination compound known to emit phosphorescence such as an Ir complex, a Pt complex, an Re complex, a Cu complex, a Eu complex, or an Rh complex. The Ir complex (Ir coordination compound) known to emit strong phosphorescence is more preferable. Further, plural kinds of phosphorescent materials may be incorporated into a light-emitting layer for the purposes of causing the light-emitting layer to effect light emission of multiple colors and aiding excitons or charge transfer.

When an organic layer containing the compound of the present invention is produced, a vacuum evaporation method, a casting method, an application method, a spin coating method, an ink jet method, or the like may be employed.

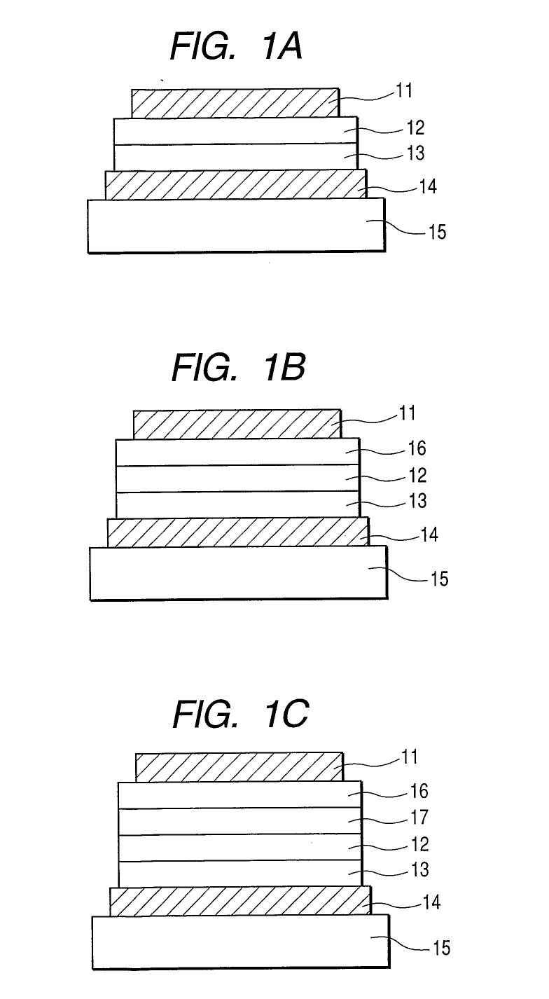

FIGS. 1A, 1B and 1C are schematic views showing basic structures of the device in accordance with the present invention.

As shown in FIGS. 1A, 1B and 1C, an organic EL device generally includes a transparent substrate 15; a transparent electrode 14 having a thickness of 50 to 200 nm on the transparent substrate 15; a plurality of organic film layers on the transparent electrode 14; and a metal electrode 11 to sandwich the plurality of organic film layers between the transparent electrode 14 and the metal electrode 11.

FIG. 1A shows an example in which the organic layers are composed of a light-emitting layer 12 and a hole-transporting layer 13. As the transparent electrode 14, ITO having a large work function is used, so that holes can be easily injected from the transparent electrode 14 to the hole-transporting layer 13. For the metal electrode 11, a metal material having a small work function such as aluminum, magnesium, or an alloy thereof is used, so that electrons can be easily injected to the organic layers.

For the light-emitting layer 12, the compound of the present invention is used. For the hole-transporting layer 13, there may be used those materials having electron-donating property, for example, a triphenyldiamine derivative typified by α-NPD.

The device having the structure described above exhibits electric rectification property. When an electric field is applied thereto with the metal electrode 11 being used as a cathode and the transparent electrode 14 being used as an anode, electrons are injected from the metal electrode 11 to the light-emitting layer 12, while holes are injected from the transparent electrode 14.

The injected holes and electrons are recombined in the light-emitting layer 12 to generate excitons, thereby effecting light emission. At this time, the hole-transporting layer 13 serves as an electron blocking layer, so that the recombination efficiency at an interface between the light-emitting,layer 12 and the hole-transporting layer 13 increases to thereby increase the emission efficiency.

In FIG. 1B, an electron-transporting layer 16 is further provided between the metal electrode 11 and the light-emitting layer 12 of the device shown in FIG. 1A. A light-emitting function and electron/hole transporting functions are separated in this manner to attain a more effective carrier blocking structure, whereby the emission efficiency is increased. For the electron-transporting layer 16, there may be used, for example, an oxadiazole derivative or the like.

Further, as shown in FIG. IC, a four-layer structure may preferably be adopted which is composed of the hole-transporting layer 13, the light-emitting layer 12, an exciton diffusion-prevention layer 17, and the electron-transporting layer 16 stacked in the mentioned order from the side of the transparent electrode 14 as the anode, and the metal electrode 11 further stacked thereon.

### EXAMPLES

Hereinafter, the present invention will be described specifically by way of examples. However, the present invention is not limited to these examples.
<Synthesis of Reaction Intermediate> 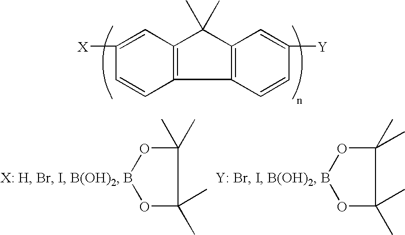
(X and Y each independently represent the above group, and n represents an integer of 1 to 5)

First, 2-halogeno-9H-fluorene and 2,7-dihalogeno-9H-fluorene were synthesized with reference to Bull. Chem. Soc. Jpn. 62 (1989) 439. The resultant compounds were subjected to dimethylation at position 9 of fluorene in DMF using CH₃Cl and NaOCH₃. Furthermore, the resultant 2-halogeno-9-dimethylfluorene and 2,7-dihalogeno-9-dimethylfluorene were subjected to synthesis of boric acid or pinacol borate. The synthesis was performed with reference to ORGANIC SYNTHESES VIA BORANES Volume 3.

The resultant compounds were subjected to an appropriate combination of the following reactions to thereby synthesize the intermediate. That is, a combination of Suzuki coupling (ORGANIC SYNTHESES VIA BORANES Volume 3) and halogenation (Bull. Chem. Soc. Jpn. 62 (1989) 439) was employed.

The compound of the present invention can be synthesized by subjecting an appropriate combination of the reaction intermediate (fluorene derivative), a halogenated benzene derivative, and a benzene boric acid derivative to a Suzuki coupling reaction.

### Example 1

### Synthesis of Exemplified Compound No. X-25

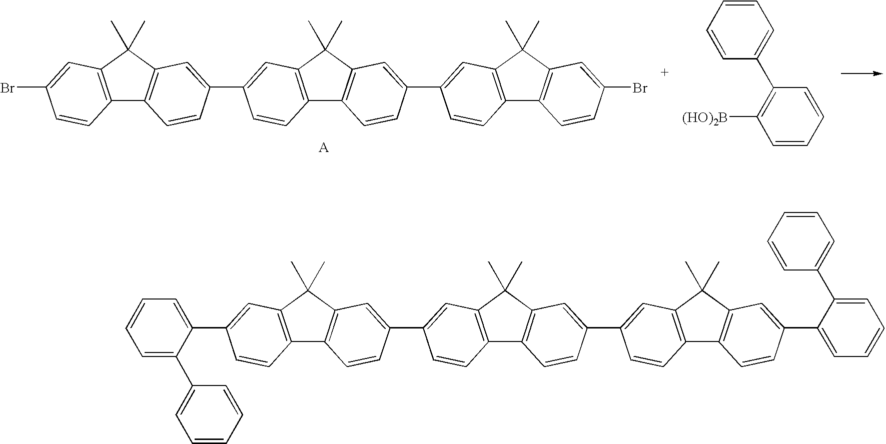

1 g (1.35 mmole) of Compound A, 672 mg (3.39 mmole) of 2-biphenylboric acid, 156 mg of Pd(PPh₃)₄, 20 ml of toluene, 10 ml of ethanol, and 20 ml of a 2M aqueous solution of sodium carbonate were fed into a 100-ml round-bottomed flask, and the whole was stirred at 80° C. for 8 hours in a stream of nitrogen. After the completion of the reaction, the resultant was extracted with toluene, and the organic layer was dried with magnesium sulfate. After that, the drying agent was filtered and the solvent was distilled off. The residue was dissolved into chloroform, and the solution was separated and purified by means of alumina column chromatography, followed by .recrystallization from toluene. The resultant crystal was vacuum-dried at 120° C., and the resultant was sublimated and purified to give 700 mg of Exemplified Compound No. X-25 (58% yield).

882.4 as M+ of the compound was confirmed by means of Matrix Assisted Laser Desorption/Ionization-Time of Flight Mass Spectrometry (MALDI-TOF MS).

In addition, the structure of the compound was identified by NMR measurement.

¹H NMR (CDCl₃, 400 MHz) σ (ppm): 7.82 (d, 4H), 7.77 (d, 4H), 7.69-7.62 (m, 20H), 7.57-7.53 (m, 4H), 7.49-7.43 (m, 12H), 7.29 (dd, 4H), 7.20-7.15 (m, 20H), 7.02 (d, 4H), 1.63 (s, 6H), 1.31 (s, 12H)

Further, the compound had a glass transition temperature of 154° C.

### Example 2

In this example, a device having three organic layers shown in FIG. 1B was used as a device structure.

ITO (as the transparent electrode 14) having a thickness of 100 nm was patterned on a glass substrate (as the transparent substrate 15). The following organic layers and electrode layers were successively formed on the ITO substrate by means of vacuum evaporation according to resistive heating .in a vacuum chamber having a pressure of 10⁻⁵ Pa such that the opposing electrode area was 3 mm². Hole-transporting layer 13 (50 nm): α-NPD Light-emitting layer 12 (50 nm): [Host] Exemplified Compound No. X-25, [Guest] Ir(4mopiq)₃ (weight ratio: 4%) and Ir(bq)₃ (weight ratio: 8%) Electron-transporting layer 16 (50 nm): Bphen (manufactured by DOJINDO LABORATORIES) Metal electrode layer 1 (1 nm): KF Metal electrode layer 2 (130 nm): Al

The current-voltage characteristics of the EL device were measured by using a microammeter 4140B (manufactured by Hewlett-Packard Development Company), and the emission luminance thereof was measured by using a BM7 (manufactured by Topcon Corporation).

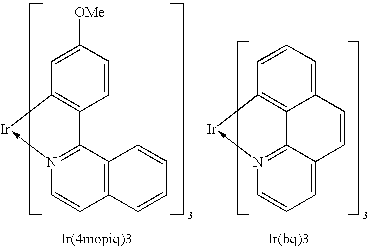

The device of this example had an efficiency of 14.6 cd/A, 14.0 lm/W (600 cd/m²). Further, the device showed a current value of 610 mA/cm² when a voltage of 8 V was applied. When the device was continuously energized at 100 mA/cm², it took 290 hours to reduce an initial luminance of 8090 cd/m² in half.

### Comparative Example 1

A device was produced following the same procedure as in Example 2 with the exception that CBP shown below was used instead of Exemplified Compound No. X-25.

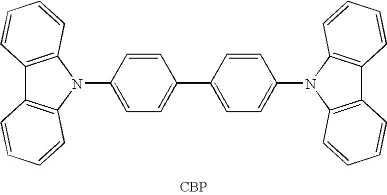

The device of this example had an efficiency of 17.2 cd/A, 12.2 lm/W (600 cd/m²). In addition, the device showed a current value of 113 mA/cm² when a voltage of 8 V was applied. When the device was continuously energized at 100 mA/cm², it took 140 hours to reduce an initial luminance of 8010 cd/m² in half.

### Comparative Example 2

A device was produced following the same procedure as in Example 2 with the exception that DB3FL shown below was used instead of Exemplified Compound No. X-25.

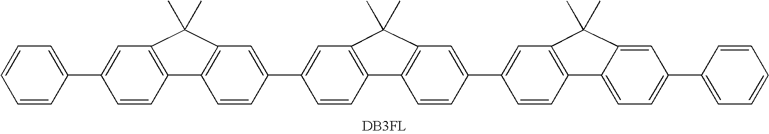

The device of this example had an efficiency of 14.3 cd/A, 14.0 lm/W (600 cd/m²). In addition, the device showed a current value of 720 mA/cm² when a voltage of 8 V was applied. When the device was continuously energized at 100 mA/cm², it took 265 hours to reduce an initial luminance of 7953 cd/m² in half. Table 1 shows those results.

TABLE 1

| | Light- | Glass | Efficiency | Current | Half- |
|---|---|---|---|---|---|
| | emitting | Transition | (lm/W) | value | value |
| | layer | temperature | at | (mA/cm²) | time |
| | host | (° C.) | 600 cd/m² | at 8 V | (h) |
| Ex. 2 | X-25 | 154 | 14.0 | 610 | 290 |
| Comp. | CBP | 115 | 12.2 | 113 | 140 |
| Ex. 1 | | | | | |
| Comp. | DB3FL | 138 | 14.0 | 720 | 265 |
| Ex. 2 | | | | | |

As shown in Table 1, the compound of the present invention has a glass transition temperature higher than those of CBP and DB3FL. In addition, the organic EL device using the compound of the present invention for the host of the light-emitting layer is an excellent device which has a power efficiency higher than that of the device using CBP and a half life about twice that of the- device using CBP. In addition, the organic EL device using the compound of the present invention shows a current value about 5 times that of the device using CBP at the same voltage value. Therefore, the instant organic EL device is extremely excellent also because it can be driven at a low voltage.

### Example 3

### Synthesis of Exemplified Compound No. X-23

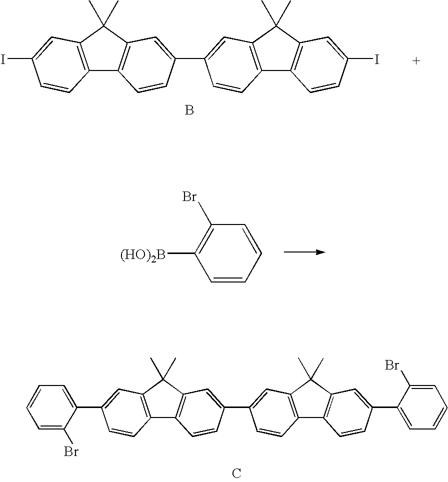

2 g (3.13 mmole) of Compound B, 1.38 g (6.89 mmole) of 2-bromophenylboric acid, 400 mg of Pd(PPh₃)₄, 20 ml of toluene, 10 ml of ethanol, and 20 ml of a 2M aqueous solution of sodium carbonate were fed into a 100-ml round-bottomed flask, and the whole was stirred at 80° C. for 4 hours in a stream of nitrogen. After the completion of the reaction, the resultant was extracted with toluene, and the organic layer was dried with magnesium sulfate. After that, the drying agent was filtered and the solvent was distilled off. The residue was dissolved into chloroform, and the solution was separated and purified by means of silica gel chromatography, followed by recrystallization from toluene, to thereby give 1.37 g of Compound C (63% yield).

694.1 as M+ of the compound was observed by means of Matrix Assisted Laser Desorption/Ionization-Time of Flight Mass Spectrometry (MALDI-TOF MS).

In addition, the structure of the compound was identified by NMR measurement.

¹H NMR (CDCl3, 400 MHz) σ (ppm): 7.81. (m, 4H), 7.69 (m, 6H), 7.53 (d., 2H), 7.40 (m, 6H), 7.02 (m, 2H), 1.61 (s, 12H)

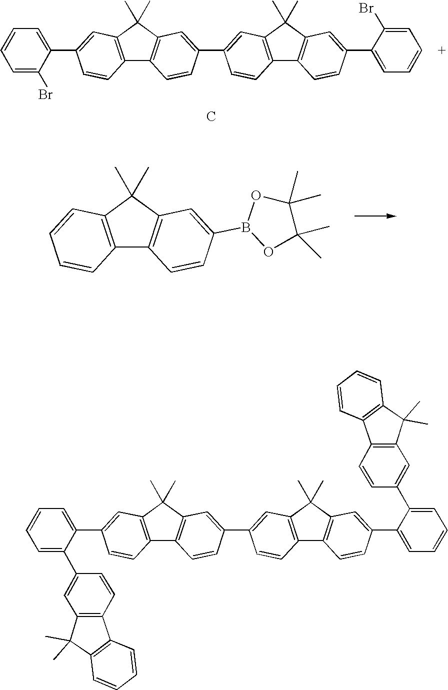

1 g (1.44 mmole) of Compound C, 1.01 g (3.16 mmole) of pinacol 2-(9,9-dimethyl)-fluoreneborate, 85 mg of Pd(PPh₃)₄, 20 ml of toluene, 10 ml of ethanol, and 20 ml of a 2M aqueous solution of sodium carbonate were fed into a 100-ml round-bottomed flask, and the whole was stirred at 80° C. for 4 hours in a stream of nitrogen. After the completion of the reaction, the resultant was extracted with toluene, and the organic layer was dried with magnesium sulfate. After that, the drying agent was filtered and the solvent was distilled off. The residue was dissolved into chloroform, and the solution was separated and purified by means of alumina column chromatography, followed by recrystallization from toluene. The resultant crystal was vacuum-dried at 120° C., and the resultant was sublimated and purified to give 718 mg of Exemplified Compound No. X-23 (54% yield).

922.5 as M+ of the compound was observed by means of Matrix Assisted Laser Desorption/Ionization-Time of Flight Mass Spectrometry (MALDI-TOF MS).

In addition, the structure of the compound was identified by NMR measurement.

¹H NMR (CDCl₃, 400 MHz) σ (ppm): 7.67 (m, 2H), 7.63 (m, 2H), 7.59-7.52 (m, 12H), 7.46 (m, 4H), 7.32-7.20 (m, 10H), 7.12 (d, 4H), 1.26 (s, 12H), 1.22 (s, 12H)

Further, the compound had a glass transition temperature of 170° C.

### Example 4

### Synthesis of Exemplified Compound No. X-24

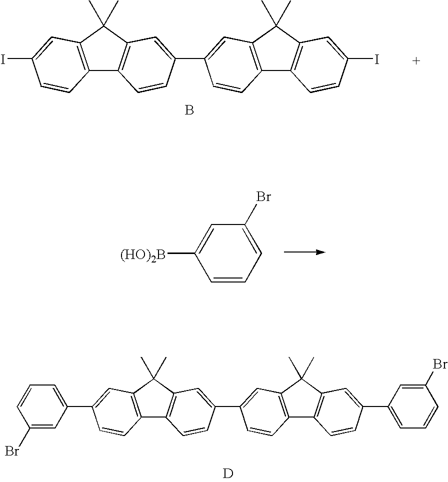

2 g (3.13 mmole) of Compound B, 1.38 mg (6.89 mmole) of 3-bromophenylboric acid, 400 mg of Pd(PPh₃)₄, 20 ml of toluene, 10 ml of ethanol, and 20 ml of a 2M aqueous solution of sodium carbonate were fed into a 100-ml round-bottomed flask, and the whole was stirred at 80° C. for 4 hours in a stream of nitrogen. After the completion of the reaction, the resultant was extracted with toluene, and the organic layer was dried with magnesium sulfate. After that, the drying agent was filtered and the solvent was distilled off. The residue was dissolved into chloroform, and the solution was separated and purified by means of alumina column chromatography, followed by recrystallization from toluene, to thereby give 1.57 g of Compound D (72% yield).

694.1 as M+ of the compound was observed by means of Matrix Assisted Laser Desorption/Ionization-Time of Flight Mass Spectrometry (MALDI-TOF MS).

In addition, the structure of the compound was identified by NMR measurement.

¹H NMR (CDCl3, 400 MHz) σ (ppm): 7.83 (d, 6H), 7.71-7.56 (m, 10H), 7.49 (m, 2H), 7.34 (t, 4H), 1.62 (s, 12H)

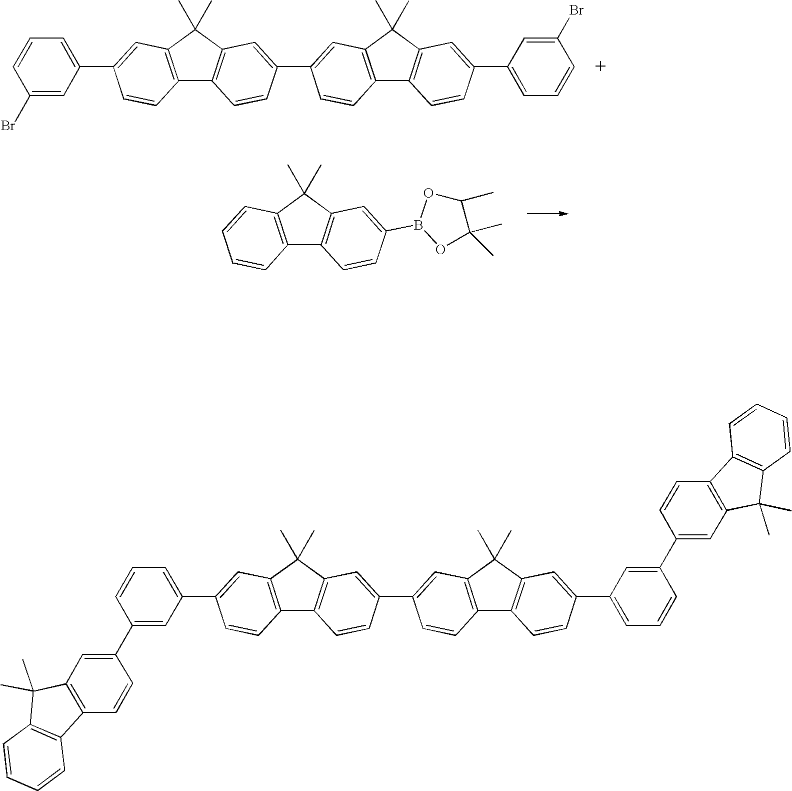

1 g (1.44 mmole) of Compound D, 1.01 g (3.16 mmole) of pinacol 2-(9,9-dimethyl)-fluoreneborate, 85 mg of Pd(PPh₃)₄, 20 ml of toluene, 10 ml of ethanol, and 20 ml of a 2M aqueous solution of sodium carbonate were fed into a 100-ml round-bottomed flask, and the whole was stirred at 80° C. for 4 hours in a stream of nitrogen. After the completion of the reaction, the resultant was extracted with toluene, and the organic layer was dried with magnesium sulfate. After that, the drying agent was filtered and the solvent was distilled off. The residue was dissolved into chloroform, and the solution was separated and purified by means of alumina column chromatography, followed by recrystallization from toluene. The resultant crystal was vacuum-dried at 120° C., and the resultant was sublimated and purified to give 884 mg of Exemplified Compound No. X-24 (64% yield).

922.5 as M+ of the compound was observed by means of Matrix Assisted Laser Desorption/Ionization-Time of Flight Mass Spectrometry (MALDI-TOF MS).

In addition, the structure of the compound was identified by NMR measurement.

¹H NMR (CDCl3, 400 MHz) σ (ppm): 7.93 (m, 2H), 7.85 (m, 6H), 7.81-7.43 (m, 18H), 7.58 (m, 4H), 7.47 (m, 2H), 7.35 (d, 4H), 1.64 (s, 12H), 1.56 (s, 12H)

Further, the compound had a glass transition temperature of 151° C.

### Example 5

### Synthesis of Exemplified Compound No. X-31

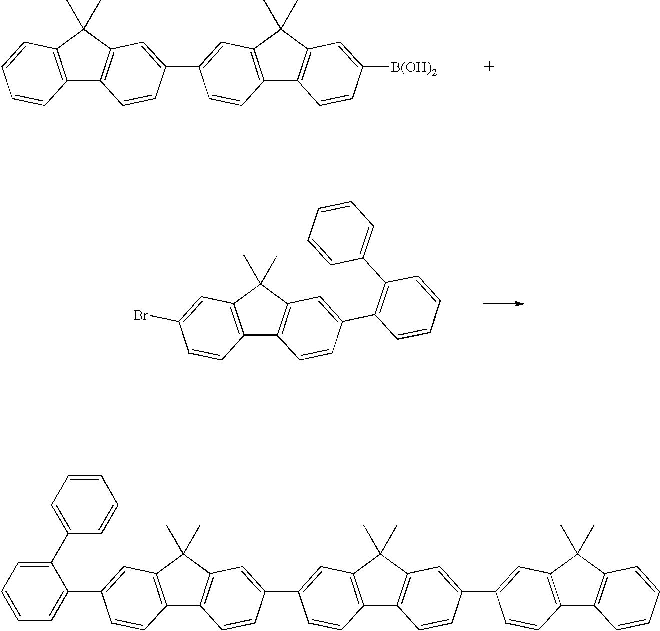

1 g (2.35 mmole) of 2-biphenyl-2-yl-7-bromo-9,9-dimethyl-9H-fluorene, 1,161 mg (2.70 mmole) of 9,9,9′,9′-tetramethyl-9H,9′H-[2,2′]bifluorenyl-7-boric acid, 90 mg of Pd(PPh₃)₄, 20 ml of toluene, 10 ml of ethanol, and 20 ml of a 2M aqueous solution of sodium carbonate were fed into a 100-ml round-bottomed flask, and the whole was stirred at 80° C. for 8 hours in a stream of nitrogen. After the completion of the reaction, the resultant was extracted with toluene, and the organic layer was dried with magnesium sulfate. After that, the drying agent was filtered and the solvent was distilled off. The residue was dissolved into chloroform, and the solution was separated and purified by means of alumina column chromatography, followed by recrystallization from toluene. The resultant crystal was vacuum-dried at 120° C., and the resultant was sublimated and purified to give 1 mg of Exemplified Compound No. X-31 (68% yield).

730.4 as M+ of the compound was observed by means of Matrix Assisted Laser Desorption/Ionization-Time of Flight Mass Spectrometry (MALDI-TOF MS).

In addition, the structure of the compound was identified by NMR measurement.

¹H NMR (CDCl3, 400 MHz) a (ppm): 7.81 (m, 5H), 7.68 (m, 9H), 7.56 (m, 1H), 7.46 (m, 4H), 7.34 (m, 3H), 7.18 (m, 5H), 7.03 (m, 1H), 1.64 (s, 6H), 1.58 (s, 6H), 1.31 (s, 6H)

Further, the compound had a glass transition temperature of 141° C.

Table 2 summarizes the physical property values of Examples 1, 3, 4, and 5, and Comparative Examples 1 and 2 through the differential scanning calorimetry (DSC).

The DSC was performed by means of a Pyris DSC1 manufactured by PerkinElmer. A glass transition temperature measured by increasing the temperature at 20(° C./min) after the formation of a glass state was adopted as a glass transition temperature. The process of temperature decrease from the melting point was measured at 40(° C./min).

A material whose glass transition temperature had not been observed in a cooling process by a DSC apparatus was heated to a temperature higher by 10° C. than its melting point, and was then quenched with liquid nitrogen to form a glass state.

TABLE 2

| | | a) Glass | | Temperature | | Recrystallization |
|---|---|---|---|---|---|---|
| | | transition | b) Recrystallization | difference | Melting | in |
| | | temperature | temperature | (° C.) | point | cooling process |
| | Compound | (° C.) | (° C.) | [a) − b)] | (° C.) | (° C.) |
| Comp. | CBP | 115 | 150 | 35 | 285 | 283 |
| Ex. 1 | | | | | | |
| Comp. | DB3FL | 138 | 184 | 46 | 308 | 306 |
| Ex. 2 | | | | | | |
| Ex. 1 | X-25 | 154 | 236 | 82 | 340 | Not observed |
| Ex. 3 | X-23 | 170 | 292 | 122 | 373 | Not observed |
| Ex. 4 | X-24 | 151 | 240 | 89 | 327 | Not observed |
| Ex. 5 | X-31 | 141 | 330 | 189 | 254 | Not observed |

As shown in Table 2, the compounds of the present invention each have a larger difference between the glass transition temperature and the recrystallization temperature in a heating process by DSC under the same conditions than that of each of Comparative Example 1 and Comparative Example 2. Each of the compounds of the present invention was observed to show a temperature difference of slightly less than twice to slightly more than four times that of each of Comparative Examples 1 and 2. On the other hand, quick crystallization was observed in each of CBP and DB3FL in a cooling process from the melting point, while each of the compounds of the present invention was observed to reach its glass transition temperature without being crystallized, to thereby form a glass state. These findings suggest that each of the compounds of the present invention can form an amorphous state more stable than those of CBP and DB3FL. Further, it can also be said that each of the compounds of the present invention is advantageous to formation of an amorphous film.

It can be said that the compound of the present invention is advantageous to the formation of an amorphous film because it has an aryl group, which is not present in DB3FL, provided in a sideward direction from the molecular major axis, and the compound is very excellent because of its improved amorphous property.

### Example 6

### Synthesis of Exemplified Compound No. X-1

Exemplified Compound No. X-1 can be synthesized following the same procedure as in Example 3 with the exception that 2,7-diiode-(9,9-dimethyl)-fluorene is used instead of Compound B of Example 3.

### Example 7

### Synthesis of Exemplified Compound No. X-3

Exemplified Compound No. X-3 can be synthesized following the same procedure as in Example 4 with the exception that 2,7-diiode-(9,9-dimethyl)-fluorene is used instead of Compound B of example 4.

### Example 8

### Synthesis of Exemplified Compound No. X-5

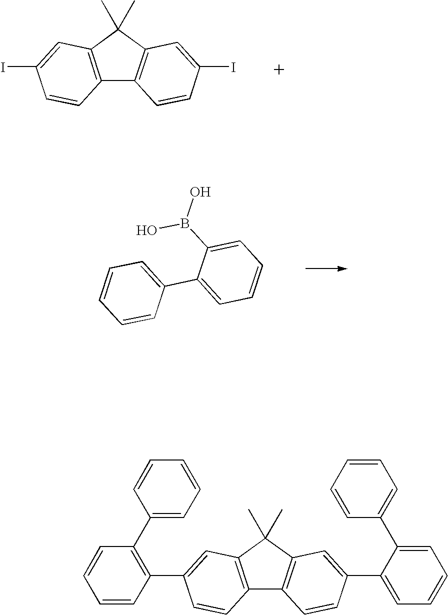

1.27 g (2.8 mmole) of 2,7-diiode-(9,9-dimethyl)-fluorene, 1.24 g (6.26 mmole) of 2-biphenylboric acid, 328 mg of Pd(PPh₃)₄, 20 ml of toluene, 10 ml of ethanol, and 20 ml of a 2M aqueous solution of sodium carbonate were fed into a 100-ml round-bottomed flask, and the whole was stirred at 80° C. for 8 hours in a stream of nitrogen. After the completion of the reaction, the resultant was extracted with toluene, and the organic layer was dried with magnesium sulfate. After that, the drying agent was filtered and the solvent was distilled off. The residue was dissolved into chloroform, and the solution was separated and purified by means of alumina column chromatography, followed by recrystallization from toluene. The resultant crystal was vacuum-dried at 120° C., and the resultant was sublimated and purified to give 925 mg of Exemplified Compound No. X-5 (65% yield).

498.2 as M+ of the compound was observed by means of Matrix Assisted Laser Desorption/Ionization-Time of Flight Mass Spectrometry (MALDI-TOF MS).

In addition, the structure of the compound was identified by NMR measurement.

¹H NMR (CDCl₃, 400 MHz) σ (ppm):. 7.59 (d, 2H), 7.52 (m, 2H), 7.44-7.39 (m, 6H), 7.24 (dd, 2H), 7.22-7.11 (m, 10H), 6.94 (d, 2H), 0.97 (s, 6H)

Further, the compound had a glass transition temperature of 80° C.

### Example 9

### Synthesis of Exemplified Compound No. X-6

Exemplified Compound No. X-6 can be synthesized following the same procedure as in Example 8 with the exception that 3-biphenylboric acid is used instead of 2-biphenylboric acid of Example 8.

### Example 10

### Synthesis of Exemplified Compound No. X-8

Exemplified Compound No. X-8 can be synthesized following the same procedure as in Example 8 with the exception that 2,5-diphenylbenzeneboric acid is used instead of 2-biphenylboric acid of Example 8.

### Example 11

### Synthesis of Exemplified Compound No. X-12

Exemplified Compound No. X-12 can be synthesized following the same procedure as in Example 1 with the exception that Compound B is used instead of Compound A of Example 1.

### Example 12

### Synthesis of Exemplified Compound No. X-13

Exemplified Compound No. X-13 can be synthesized following the same procedure as in Example 11 with the exception that 3-biphenylboric acid is used instead of 2-biphenylboric acid of Example 11.

### Example 13

### Synthesis of Exemplified Compound No. X-14

Exemplified Compound No. X-14 can be synthesized following the same procedure as in Example 11 with the exception that 2,5-diphenylbenzeneboric acid is used instead of 2-biphenylboric acid of Example 11.

### Example 14

### Synthesis of Exemplified Compound No. X-15

Exemplified Compound No. X-15 can be synthesized following the same procedure as in Example 10 with the exception that Compound B is used instead of 2,7-diiode-(9,9-dimethyl)-fluorene of Example 10.

### Example 15

### Synthesis of Exemplified Compound No. X-19

Exemplified Compound No. X-19 can be synthesized following the same procedure as in Example 6 with the exception that Compound B is used instead of 2,7-diiode-(9,9-dimethyl)-fluorene of Example 6.

### Example 16

### Synthesis of Exemplified Compound No. X-20

Exemplified Compound No. X-20 can be synthesized following the same procedure as in Example 7 with the exception that Compound B is used instead of 2,7-diiode-(9,9-dimethyl)-fluorene of Example 7.

### Example 17

### Synthesis of Exemplified Compound No. X-22

Exemplified Compound No. X-22 can be synthesized following the same procedure as in Example 14 with the exception that 3-(9,9-dimethyl)fluorenyl-5-phenylbenzeneboric acid is used instead of 3,5-diphenylbenzeneboric acid in Example 14.

### Example 18

### Synthesis of Exemplified Compound No. X-26

Exemplified Compound No. X-26 can be synthesized following the same procedure as in Example 1 with the exception that 3-biphenylboric acid is used instead of 2-biphenylboric acid in Example 1.

### Example 19

### Synthesis of Exemplified Compound No. X-27

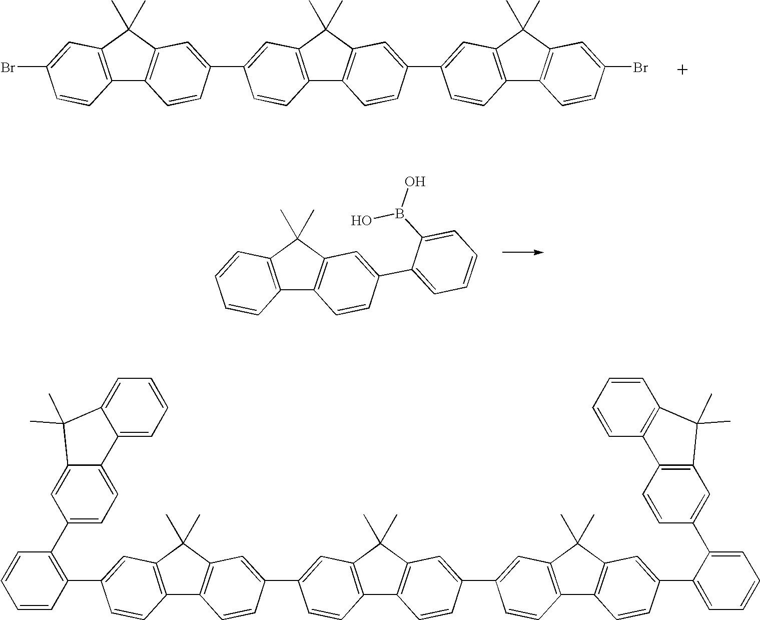

956 mg (1.3 mmole) of Compound A, 900 mg (2.86 mmole) of 2-fluorenylphenylboric acid, 380 mg of Pd(PPh₃)₄, 20 ml of toluene, 10 ml of ethanol, and 20 ml of a 2M aqueous solution of sodium carbonate were fed into a 100-ml round-bottomed flask, and the whole was stirred at 80° C. for 8 hours in a stream of nitrogen. After the completion of the reaction, the resultant was extracted with toluene, and the organic layer was dried with magnesium sulfate. After that, the drying agent was filtered and the solvent was distilled off. The residue was dissolved into chloroform, and the solution was separated and purified by means of alumina column chromatography, followed by recrystallization from toluene. The resultant crystal was vacuum-dried at 120° C. to give 980 mg of Exemplified Compound No. X-27 (67% yield).

1131.5 as M+ of the compound was observed by means of Matrix Assisted Laser Desorption/Ionization-Time of Flight Mass Spectrometry (MALDI-TOF MS).

In addition, the structure of the compound was identified by NMR measurement.

¹H NMR (CDCl₃, 400 MHz) σ (ppm): 7.78 (d, 2H), 7.70 (d, 2H), 7.66-7.56 (m, 18H), 7.48-7.45 (m, 4H), 7.33-7.21 (m, 10H), 7.14 (m, 4H), 1.60 (s, 6H), 1.28 (s, 12H), 1.23 (s, 12H)

### Example 20

### Synthesis of Exemplified Compound No. X-28

Exemplified Compound No. X-28 can be. synthesized following the same procedure as in Example 4 with the exception that Compound A is used instead of Compound B in Example 4.

### Example 21

### Synthesis of Exemplified Compound No. X-29

Exemplified Compound No. H-29 can be synthesized following the same procedure as in Example 1 with the exception that 1,1′:4′,1″-t-riphenyl-3-boric acid is used instead of 2-phenylboric acid in Example 1.

### Example 22

### Synthesis of Exemplified Compound No. X-30

Exemplified Compound No. X-30 can be synthesized following the same procedure as in Example 1 with the exception that 1,1′:4′,1″-t-riphenyl-2-boric acid is used instead of 2-phenylboric acid in Example 1.

### Example 23

### Synthesis of Exemplified Compound No. X-31

Exemplified Compound No. X-31 can be synthesized following the same procedure as in Example 1 with the exception that Compound D1 is used instead of Compound A of Example 1 and the amount of 2-biphenylboric acid is 1 equivalent.

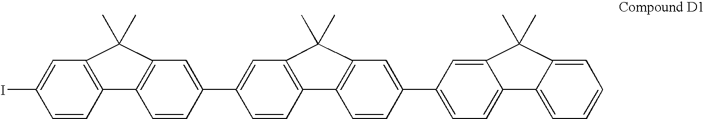

### Example 24

### Synthesis of Exemplified Compound No. X-32

Exemplified Compound No. X-32 can be synthesized following the same procedure as in Example 23 with the exception that 3-biphenylboric acid is used instead of 2-biphenylboric acid of Example 23.

### Example 25

### Synthesis of Exemplified Compound No. X-33

Exemplified Compound No. X-33 can be synthesized following the same procedure as in Example 3 with the exception that Compound D1 is used instead of Compound B of Example 3 and the amount of pinacol 2-(9,9-dimethyl)-fluoreneborate is 1 equivalent.

### Example 26

### Synthesis of Exemplified Compound No. H-34

Exemplified Compound No. X-34 can be synthesized following the same procedure as in Example 4 with the exception that Compound D1 is used instead of Compound B in Example 4 and the amount of pinacol 2-(9,9-dimethyl)-fluorenebbrate is 1 equivalent.

### Example 27

### Synthesis of Exemplified Compound No. X-39

Exemplified Compound No. X-39 can be synthesized following the same procedure as in Example 23 with the exception that 3,5-diphenylbenzeneboric acid is used instead of 2-biphenylboric acid in Example 23.

### Example 28

### Synthesis of Exemplified Compound No. X-48

Exemplified Compound No. X-48 can be synthesized following the same procedure as in Example 23 with the exception that Compound E is used instead of Compound D1 in Example 23.

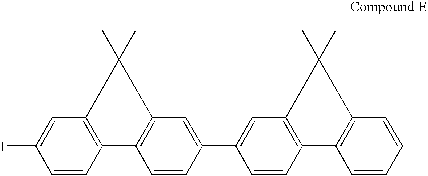

### Example 29

### Synthesis of Exemplified Compound No. X-49

Exemplified Compound No. X-49 can be synthesized following the same procedure as in Example 24 with the exception that Compound E is used instead of Compound D1 of Example 24.

### Example 30

### Synthesis of Exemplified Compound No. X-51

Exemplified Compound No. X-51 can be synthesized following the same procedure as in Example 27 with the exception that Compound E is used instead of Compound D1 in Example 27.

### Example 31

### Synthesis of Exemplified Compound No. X-57

Exemplified Compound No. X-57 can be synthesized following the same procedure as in Example 25 with the exception that Compound E is used instead of Compound D1 in Example 25.

### Example 32

### Synthesis of Exemplified Compound No. X-58

Exemplified Compound No. X-58 can be synthesized following the same procedure as in Example 26 with the exception that Compound E is used instead of Compound D1 in Example 26.

### Example 33

### Synthesis of Exemplified Compound No. X-61

Exemplified Compound No. X-61 can be synthesized following the same procedure as in Example 28 with the exception that Compound F is used instead of Compound E in Example 28 and Compound G is used instead of 2-biphenylbenzeneboric acid in Example 28.

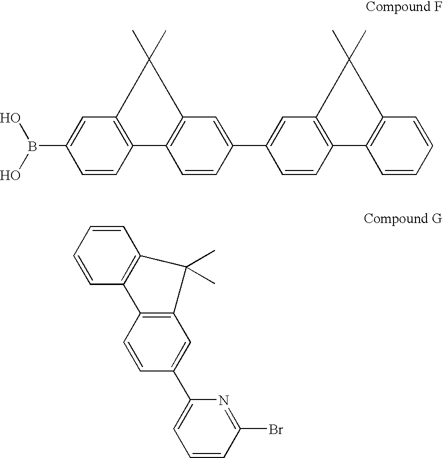

### Example 34

### Synthesis of Exemplified Compound No. X-62

Exemplified Compound No. X-62 can be synthesized following the same procedure as in Example 33 with the exception that Compound H is used instead of Compound G in Example 33.

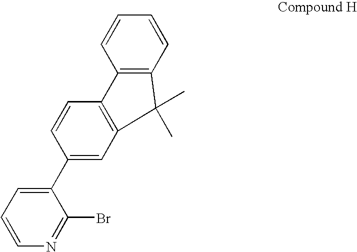

### Example 35

### Synthesis of Exemplified Compound No. X-63

Exemplified Compound No. X-63 can be synthesized following the same procedure as in Example 33 with the exception that Compound J is used instead of Compound G in example 33.

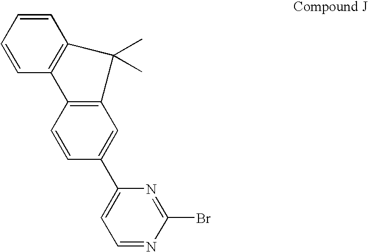

### Example 36

### Synthesis of Exemplified Compound No. X-64

Exemplified Compound No. X-64 can be synthesized following the same procedure as in Example 33 with the exception that Compound I is used instead of Compound G in Example 33.

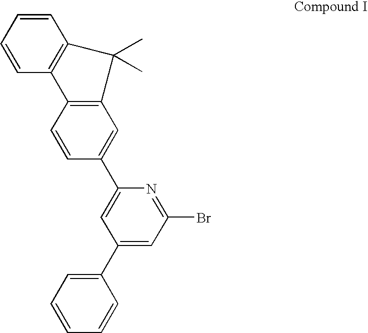

### Example 37

### Synthesis of Exemplified Compound No. X-65

Exemplified Compound No. X-65 can be synthesized following the same procedure as in Example 33 with the exception that Compound K is used instead of Compound G in Example 33.

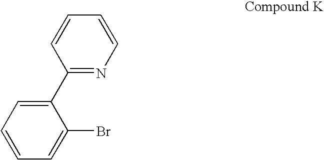

### Example 38

### Synthesis of Exemplified Compound No. X-71

Exemplified Compound No. X-71 can be synthesized following the same procedure as in Example 33 with the exception that Compound N is used instead of Compound F in Example 33 and Compound K is used instead of Compound G in Example 33.

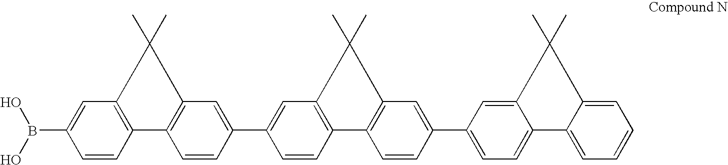

### Example 39

### Synthesis of Exemplified Compound No. X-72

Exemplified Compound No. X-72 can be synthesized following the same procedure as in Example 38 with the exception that Compound M is used instead of Compound K in Example 38.

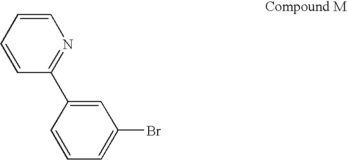

### Example 40

### Synthesis of Exemplified Compound No. X-73

Exemplified Compound No. X-73 can be synthesized following the same procedure as in Example 38 with the exception that Compound H is used instead of Compound K in Example 38.

### Example 41

### Synthesis of Exemplified Compound No. X-74

Exemplified Compound No. X-74 can be synthesized following the same procedure as in Example 38 with the exception that Compound G is used instead of Compound K in Example 38.

### Example 42

### Synthesis of Exemplified Compound No. X-78

Exemplified Compound No. X-78 can be synthesized following the same procedure as in Example 38 with the exception that Compound N1 is used instead of Compound K in Example 38.

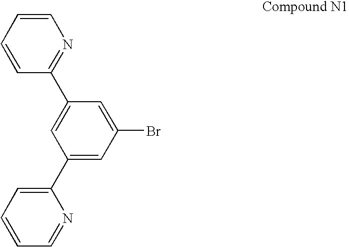

### Example 43

### Synthesis of Exemplified Compound No. X-82

Exemplified Compound No. X-82 can be synthesized following the same procedure as in Example 38 with the exception that Compound L is used instead of Compound K in Example 38.

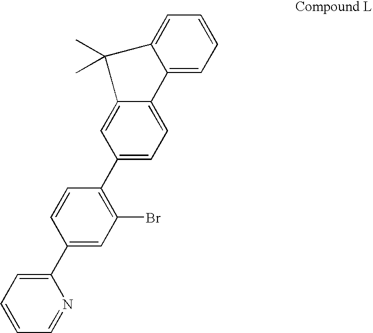

### Example 44

### Synthesis of Exemplified Compound No. X-84

Exemplified Compound No. X-84 can be synthesized following the same procedure as in Example 38 with the exception that Compound O is used instead of Compound N in Example 38 and Compound P is used instead of Compound K in Example 38.

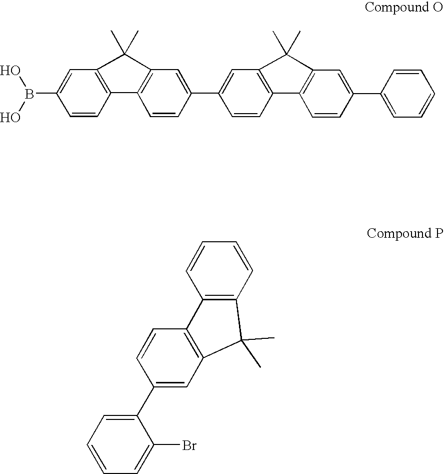

### Example 45

### Synthesis of Exemplified Compound No. X-85

Exemplified Compound No. X-85 can be synthesized following the same procedure as in Example 44 with the exception that Compound Q is used instead of Compound P in Example 44.

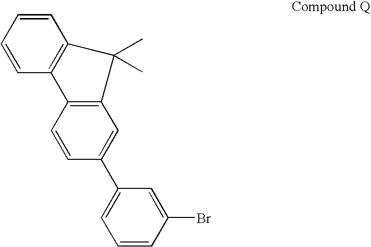

### Example 46

### Synthesis of Exemplified Compound No. X-86

Exemplified Compound No. X-86 can be synthesized following the same procedure as in Example 44 with the exception that Compound R is used instead of Compound P in Example 44.

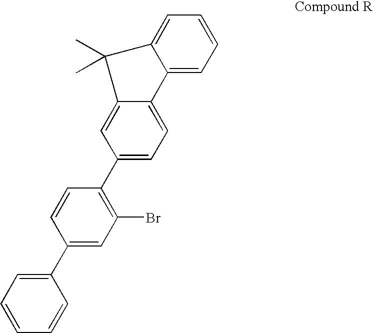

### Example 47

### Synthesis of Exemplified Compound No. X-87

Exemplified Compound No. X-87 can be synthesized following the same procedure as in Example 44 with the exception that Compound S is used instead of Compound P in Example 44.

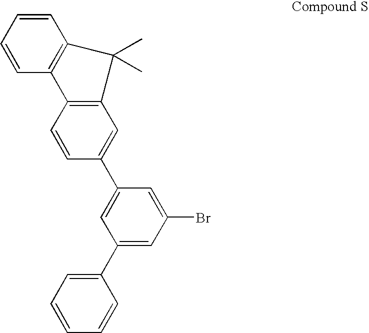

### Example 48

### Synthesis of Exemplified Compound No. X-90

Exemplified Compound No. X-90 can be synthesized following the same procedure as in Example 44 with the exception that 2-biphenyl bromide is used instead of Compound P in Example 44.

### Example 49

### Synthesis of Exemplified Compound No. X-91

Exemplified Compound No. X-91 can be synthesized following the same procedure as in Example 44 with the exception that 3-biphenyl bromide is used instead of Compound P in Example 44.

### Example 50

### Synthesis of Exemplified Compound No. X-92

Exemplified Compound No. X-92 can be synthesized following the same procedure as in Example 44 with the exception that 2,5-diphenyl bromobenzene is used instead of Compound P in Example 44.

### Example 51

### Synthesis of Exemplified Compound No. X-93

Exemplified Compound No. X-93 can be synthesized following the same procedure as in Example 44 with the exception that 3,5-diphenyl bromobenzene is used instead of Compound P in Example 44.

### Example 52

### Synthesis of Exemplified Compound No. X-97

Exemplified Compound No. X-97 can be synthesized following the same procedure as in Example 38 with the exception that Compound T is used instead of Compound N in Example 38 and Compound R is used instead of Compound K in Example 38.

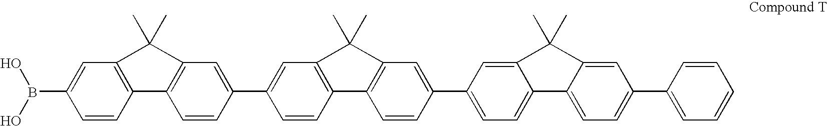

### Example 53

### Synthesis of Exemplified Compound No. X-98

Exemplified Compound No. X-98 can be synthesized following the same procedure as in Example 52 with the exception that Compound U is used instead of Compound R in Example 52.

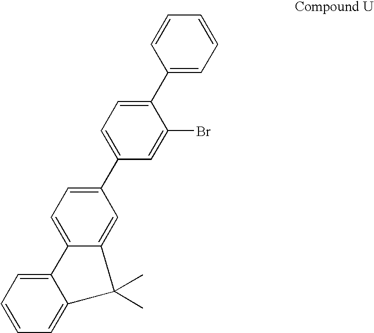

### Example 54

### Synthesis of Exemplified Compound No. X-103

Exemplified Compound No. X-103 can be synthesized following the same procedure as in Example 52 with the exception that 2,5-diphenyl bromobenzene is used instead of Compound R in Example 52.

### Example 55

### Synthesis of Exemplified Compound No. X-104

Exemplified Compound No. X-104 can be synthesized following the same procedure as in Example 52 with the exception that 3,5-diphenyl bromobenzene is used instead of Compound R in Example 52.

### Example 56

### Synthesis of Exemplified Compound No. X-108

Exemplified Compound No. X-108 can be synthesized following the same procedure as in Example 52 with the exception that 2-biphenyl bromide is used instead of Compound R in Example 52.

### Example 57

### Synthesis of Exemplified Compound No. X-109

Exemplified Compound No. X-109 can be synthesized following the same procedure as in Example 52 with the exception that 3-biphenyl bromide is used instead of Compound R in Example 52.

### Example 58

### Synthesis of Exemplified Compound No. X-110

Exemplified Compound No. X-110 can be synthesized following the same procedure as in Example 52 with the exception that Compound Q is used instead of Compound R in Example 52.

### Example 59

### Synthesis of Exemplified Compound No. X-111

Exemplified Compound No. X-111 can be synthesized following the same procedure as in Example 52 with the exception that Compound P is used instead of Compound R in Example 52.

### Example 60

### Synthesis of Exemplified Compound No. X-112

Exemplified Compound No. X-112 can be synthesized following the same procedure as in Example 52 with the exception that Compound S is used instead of Compound R in Example 52.

### Example 61

### Synthesis of Exemplified Compound No. X-113

Exemplified Compound No. X-113 can be synthesized following the same procedure as in Example 38 with the exception that Compound V is used instead of Compound N in Example 38 and Compound P is used instead of Compound K in Example 38.

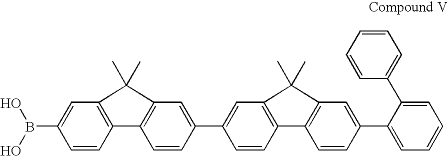

### Example 62

### Synthesis of Exemplified Compound No. X-114

Exemplified Compound No. X-114 can be synthesized following the same procedure as in Example 61 with the exception that Compound Q is, used instead of Compound P in Example 61.

### Example 63

### Synthesis of Exemplified Compound No. X-115

Exemplified Compound No. X-115 can be synthesized following the same procedure as in Example 61 with the exception that Compound S is used instead of Compound P in Example 61.

### Example 64

### Synthesis of Exemplified Compound No. X-116

Exemplified Compound No. X-116 can be synthesized following the same procedure as in Example 61 with the exception that Compound R is used instead of Compound P in Example 61.

### Example 65

### Synthesis of Exemplified Compound No. X-120

Exemplified Compound No. X-120 can be synthesized following the same procedure as in Example 61 with the exception that 2-biphenyl bromide is used instead of Compound P in Example 61.

### Example 66

### Synthesis of Exemplified Compound No. X-121

Exemplified Compound No. X-121 can be synthesized following the same procedure as in Example 61 with the exception that 2,5-diphenyl bromobenzene is used instead of Compound P in Example 61.

### Example 67

### Synthesis of Exemplified Compound No. X-122

Exemplified Compound No. X-122 can be synthesized following the same procedure as in Example 61 with the exception that 3,5-diphenyl bromobenzene is used instead of Compound P in Example 61.

### Example 68

### Synthesis of Exemplified Compound No. X-126

Exemplified Compound No. X-126 can be synthesized following the same procedure as in Example 38 with the exception that Compound W is used instead of Compound N in Example 38 and Compound R is used instead of Compound K in Example 38.

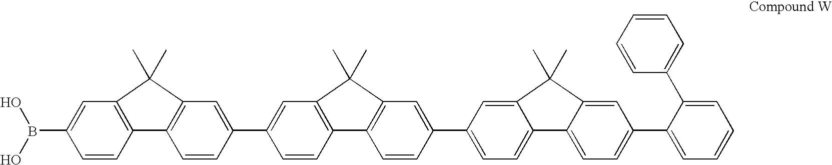

### Example 69

### Synthesis of Exemplified Compound No. X-127

Exemplified Compound No. X-127 can be synthesized following the same procedure as in Example 68 with the exception that Compound U is used instead of Compound R in Example 68.

### Example 70

### Synthesis of Exemplified Compound No. X-128

Exemplified Compound No. X-128 can be synthesized following the same procedure as in Example 68 with the exception that Compound S is used instead of Compound R in Example 68.

### Example 71

### Synthesis of Exemplified Compound No. X-132

Exemplified Compound No. X-132 can be synthesized following the same procedure as in Example 68 with the exception that 2,5-diphenyl bromobenzene is used instead of Compound R in Example 68.

### Example 72

### Synthesis of Exemplified Compound No. X-133

Exemplified Compound No. X-133 can be synthesized following the same procedure as in Example 68 with the exception that 3,5-diphenyl bromobenzene is used instead of Compound R in Example 68.

### Example 73

### Synthesis of Exemplified Compound No. X-137

Exemplified Compound No. X-137 can be synthesized following the same procedure as in Example 68 with the exception that 1,1′:4′,1″-t-riphenyl-3-bromide is used instead of Compound R in Example 68.

### Example 74

### Synthesis of Exemplified Compound No. X-138

Exemplified Compound No. X-138 can be synthesized following the same procedure as in Example 68 with the exception that Compound Q is used instead of Compound R in Example 68.

### Example 75

### Synthesis of Exemplified Compound No. X-139

Exemplified Compound No. X-139 can be synthesized following the same procedure as in Example 68 with the exception that 1,1′:4′,1″-t-riphenyl-2-bromide is used instead of Compound R in Example 68.

### Example 76

### Synthesis of Exemplified Compound No. X-140

Exemplified Compound No. X-140 can be synthesized following the same procedure as in Example 68 with the exception that Compound P is used instead of Compound R in Example 68.

### Example 77

### Synthesis of Exemplified Compound No. X-141

Exemplified Compound No. X-141 can be synthesized following the same procedure as in Example 68 with the exception that 3s-biphenyl bromide is used instead of Compound R in Example 68.

### Example 78

### Synthesis of Exemplified Compound No. X-142

Exemplified Compound No. X-142 can be synthesized following the same procedure as in Example 1 with the exception that Compound Ad is used instead of Compound A in Example 1 and Compound H is used instead of 2-biphenylboric acid in Example 1.

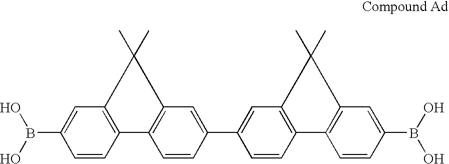

### Example 79

### Synthesis of Exemplified Compound No. X-143

Exemplified Compound No. X-143 can be synthesized following the same procedure as in Example 78with the exception that Compound G is used instead of Compound H in Example 78.

### Example 80

### Synthesis of Exemplified Compound No. X-144

Exemplified Compound No. X-144 can be synthesized following the same procedure as in Example 78 with the exception that Compound Aa is used instead of Compound H in Example 78.

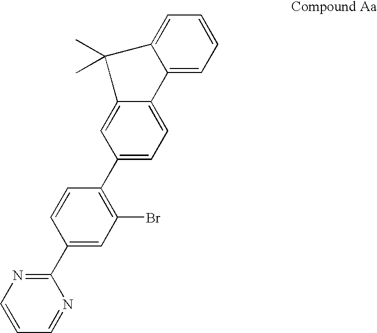

### Example 81

### Synthesis of Exemplified Compound No. X-146

Exemplified Compound No. X-146 can be synthesized following the same procedure as in Example 78 with the exception that Compound Ab is used instead of Compound H in Example 78.

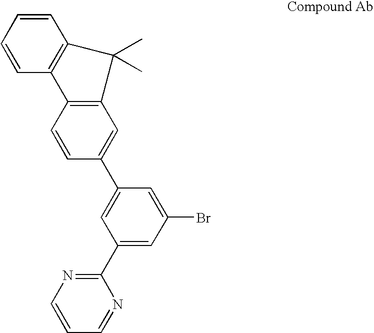

### Example 82

### Synthesis of Exemplified Compound No. X-147

Exemplified Compound No. X-147 can be synthesized following the same procedure as in Example 78 with the exception that Compound Ac is used instead of Compound H in Example 78.

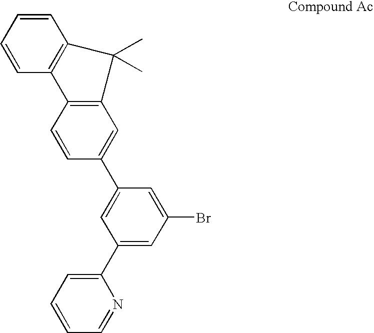

### Example 83

### Synthesis of Exemplified Compound No. X-149

Exemplified Compound No. X-149 can be synthesized following the same procedure as in Example 1 with the exception that Compound Ae is used instead of Compound A in Example 1 and Compound Aa is used instead of 2-biphenylboric acid in Example 1.

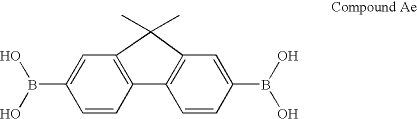

### Example 84

### Synthesis of Exemplified Compound No. X-150

Exemplified Compound No. X-150 can be synthesized following the same procedure as in Example 83 with the exception that Compound H is used instead of Compound Aa in Example 83.

### Example 85

### Synthesis of Exemplified Compound No. X-151

Exemplified Compound No. X-151 can be synthesized following the same procedure as in Example 83 with the exception that Compound G is used instead of Compound Aa in Example 83.

### Example 86

### Synthesis of Exemplified Compound No. X-152

Exemplified Compound No. X-152 can be synthesized following the same procedure as in Example 83 with the exception that Compound Ab is used instead of Compound Aa in Example 83.

### Example 87

### Synthesis of Exemplified Compound No. X-154

Exemplified Compound No. X-154 can be synthesized following the same procedure as in Example 83 with the exception that Compound Ac is used instead of Compound Aa in Example 83.

### Example 88

### Synthesis of Exemplified Compound No. X-162

Exemplified Compound No. X-162 can be synthesized following the same procedure as in Example 83 with the exception that Compound N1 is used instead of Compound Aa in Example 83.

### Example 89

### Synthesis of Exemplified Compound No. X-165

Exemplified Compound No. X-165 can be synthesized following the same procedure as in Example 83 with the exception that Compound Ag is used instead of Compound Aa in Example 83.

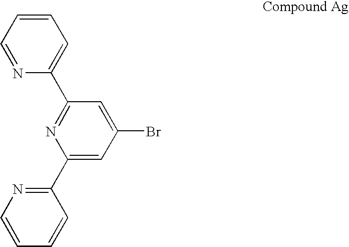

### Example 90

### Synthesis of Exemplified Compound No. X-168

Exemplified Compound No. X-168 can be synthesized following the same procedure as in Example 1 with the exception that Compound Af is used instead of Compound A in Example 1 and Compound K is used instead of 2-biphenylboric acid in Example 1.

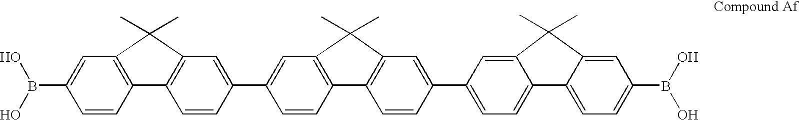

### Example 91

### Synthesis of Exemplified Compound No. X-169

Exemplified Compound No. X-169 can be synthesized following the same procedure as in Example 90 with the exception that Compound H is used instead of Compound K in Example 90.

### Example 92

### Synthesis of Exemplified Compound No. X-170

Exemplified Compound No. X-170 can be synthesized following the same procedure as in Example 90 with the exception that Compound G is used instead of Compound K in Example 90.

### Example 93

### Synthesis of Exemplified Compound No. X-176

Exemplified Compound No. X-176 can be synthesized following the same procedure as in Example 90 with the exception that Compound Ag is used instead of Compound K in Example 90.

### Example 94

### Synthesis of Exemplified Compound No. X-179

Exemplified Compound No. X-179 can be synthesized following the same procedure as in Example 90 with the exception that Compound L is used instead of Compound K in Example 90.

### Example 95

### Synthesis of Exemplified Compound No. X-181

Exemplified Compound No. X-181 can be synthesized following the same procedure as in Example 90 with the exception that Compound Ab is used instead of Compound K in Example 90.

### Example 96

### Synthesis of Exemplified Compound No. X-182

Exemplified Compound No. X-182 can be synthesized following the same procedure as in Example 90 with the exception that Compound N is used instead of Compound K in Example 90.

### Example 97

### Synthesis of Exemplified Compound No. X-183

Exemplified Compound No. X-183 can be synthesized following the same procedure as in Example 1 with the exception that Compound Ah is used instead of Compound A in Example 1; and 2,5-diphenyl bromobenzene is used instead of 2-biphenylboric acid in Example 1.

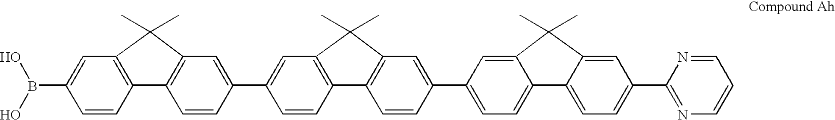

### Example 98

### Synthesis of Exemplified Compound No. X-185

Exemplified Compound No. X-185 can be synthesized following the same procedure as in Example 97 with the exception that 3,5-diphenyl bromobenzene is used instead of 2,5-diphenyl bromobenzene in Example 97.

### Example 99

### Synthesis of Exemplified Compound No. X-193

Exemplified Compound No. X-193 can be synthesized following the same procedure as in Example 97 with the exception that 2-biphenyl bromide is used instead of 2,5-diphenyl bromobenzene in Example 97.

### Example 100

### Synthesis of Exemplified Compound No. X-194

Exemplified Compound No. X-194 can be synthesized following the same procedure as in Example 97 with the exception that 3-biphenyl bromide is used instead of 2,5-diphenyl bromobenzene in Example 97.

### Example 101

### Synthesis of Exemplified Compound No. X-195

Exemplified Compound No. X-195 can be synthesized following the same procedure as in Example 97 with the exception that Compound P is used instead of 2,5-diphenyl bromobenzene in Example 97.

### Example 102

### Synthesis of Exemplified Compound No. X-196

Exemplified Compound No. X-196 can be synthesized following the same procedure as in Example 97 with the exception that Compound Q is used instead of 2,5-diphenyl bromobenzene in Example 97.

### Example 103

### Synthesis of Exemplified Compound No. X-197

Exemplified Compound No. X-197 can be synthesized following the same procedure as in Example 97 with the exception that 1,1′:4′,1″-t-riphenyl-3-bromide is used instead of 2,5-diphenyl bromobenzene in Example 97.

### Example 104

### Synthesis of Exemplified Compound No. X-198

Exemplified Compound No. X-198 can be synthesized following the same procedure as in Example 97 with the exception that 1,1′:4′,1″-t-riphenyl-2-bromide is used instead of 2,5-diphenyl bromobenzene in Example 97.

### Example 105

### Synthesis of Exemplified Compound No. X-184

Exemplified Compound No. X-184 can be synthesized following the same procedure as in Example 1 with the exception that Compound Ai is used instead of Compound A in Example 1 and 2,5-diphenyl bromobenzene is used instead of 2-biphenylboric acid in Example 1.

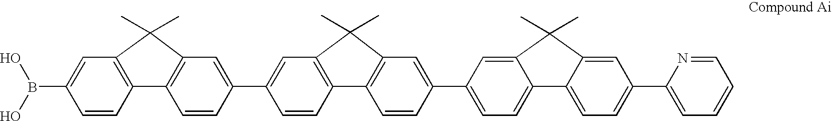

### Example 106

### Synthesis of Exemplified Compound No. X-186

Exemplified Compound No. X-186 can be synthesized following the same procedure as in Example 105 with the exception that 3,5-diphenyl bromobenzene is used instead of 2,5-diphenyl bromobenzene in Example 105.

### Example 107

### Synthesis of Exemplified Compound No. X-197

Exemplified Compound No. X-187 can be synthesized following the same procedure as in Example 105 with the exception that 2-biphenyl bromide is used instead of 2,5-diphenyl bromobenzene in Example 105.

### Example 108

### Synthesis of Exemplified Compound No. X-188

Exemplified Compound No. X-188 can be synthesized following the same procedure as in Example 105 with the exception that 3-biphenyl bromide is used instead of 2,5-diphenyl bromobenzene in Example 105.

### Example 109

### Synthesis of Exemplified Compound No. X-189

Exemplified Compound No. X-189 can be synthesized following the same procedure as in Example 105 with the exception that Compound P is used instead of 2,5-diphenyl bromobenzene in Example 105.

### Example 110

### Synthesis of Exemplified Compound No. X-190

Exemplified Compound No. X-190 can be synthesized following the same procedure as in Example 105 with the exception that Compound Q is used instead of 2,5-diphenyl bromobenzene in Example 105.

### Example 111

### Synthesis of Exemplified Compound No. X-191

Exemplified Compound No. X-191 can be synthesized following the same procedure as in Example 105 with the exception that 1,1′:4′,1″-t-riphenyl-2-bromide is used instead of 2,5-diphenyl bromobenzene in Example 105.

### Example 112

### Synthesis of Exemplified Compound No. X-192

Exemplified Compound No. X-192 can be synthesized following the same procedure as in Example 105 with the exception that 1,1′:4′,1″-t- riphenyl-3-bromide is used instead of 2,5-diphenyl bromobenzene in Example 105.

### Example 113

### Synthesis of Exemplified Compound No. X-199

Exemplified Compound No. X-199 can be synthesized following the same procedure as in Example 105 with the exception that Compound R is used instead of 2,5-diphenyl bromobenzene in Example 105.

### Example 114

### Synthesis of Exemplified Compound No. X-201

Exemplified Compound No. X-201 can be synthesized following the same procedure as in Example 1 with the exception that Compound Aj is used instead of Compound A in Example 1 and 3-biphenyl bromide is used instead of 2-biphenylboric acid in Example 1.

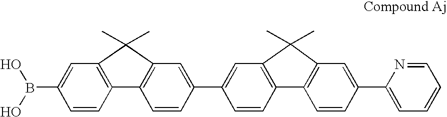

### Example 115

### Synthesis of Exemplified Compound No. X-202

Exemplified Compound No. X-202 can be synthesized following the same procedure as in Example 114 with the exception that 2-biphenyl bromide is used instead of 3-biphenyl bromide in Example 114.

### Example 116

### Synthesis of Exemplified Compound No. X-203

Exemplified Compound No. X-203 can be synthesized following the same procedure as in Example 114 with the exception that 3,5-diphenyl bromobenzene is used instead of 3-biphenyl bromide in Example 114.

### Example 117

### Synthesis of Exemplified Compound No. X-204

Exemplified Compound No. X-204 can be synthesized following the same procedure as in Example 114 with the exception that 2,5-diphenyl bromobenzene is used instead of 3-biphenyl bromide in Example 114.

### Example 118

### Synthesis of Exemplified Compound No. X-205

Exemplified Compound No. X-205 can be synthesized following the same procedure as in Example 114 with the exception that Compound Q is used instead of 3-biphenyl bromide in Example 114.

### Example 119

### Synthesis of Exemplified Compound No. X-207

Exemplified Compound No. X-207 can be synthesized following the same procedure as in Example 114 with the exception that Compound P is used instead of 3-biphenyl bromide in Example 114.

### Example 120

### Synthesis of Exemplified Compound No. X-211

Exemplified Compound No. X-211 can be synthesized following the same procedure as in Example 114 with the exception that Compound S is used instead of 3-biphenyl bromide in Example 114.

### Example 121

### Synthesis of Exemplified Compound No. X-206

Exemplified Compound No. X-206 can be synthesized following the same procedure as in Example 1 with the exception that Compound Ak is used instead of Compound A in Example 1 and Compound Q is used instead of 2-biphenylboric acid in Example 1.

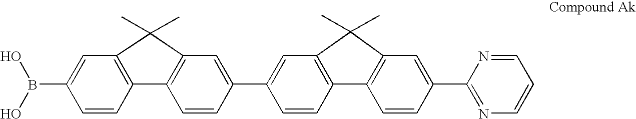

### Example 122

### Synthesis of Exemplified Compound No. X-208

Exemplified Compound No. X-208 can be synthesized following the same procedure as in Example 121 with the exception that Compound P is used instead of Compound Q in Example 121.

### Example 123

### Synthesis of Exemplified Compound No. X-210

Exemplified Compound No. X-210 can be synthesized following the same procedure as in Example 121 with the exception that Compound S is used instead of Compound Q in Example 121.

### Example 124

### Synthesis of Exemplified Compound No. X-214

Exemplified Compound No. X-214 can be synthesized following the same procedure as in Example 121 with the exception that Compound R is used instead of Compound Q in Example 121.

### Example 125

### Synthesis of Exemplified Compound No. X-215

Exemplified Compound No. X-215 can be synthesized following the same procedure as in Example 1 with the exception that 2,7-diiode-(9,9-dimethyl)-fluorene is used instead of Compound A in Example 1; and Compound Ak1 is used instead of 2-biphenylboric acid in Example 1.

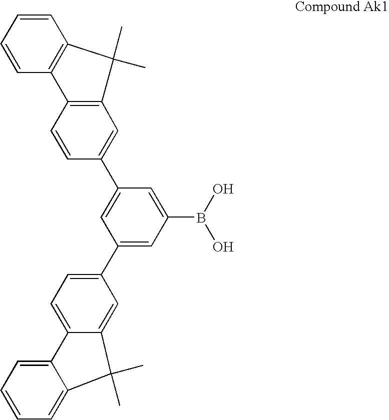

### Example 126

### Synthesis of Exemplified Compound No. X-216

Exemplified Compound No. X-216 can be synthesized following the same procedure as in Example 125 with the exception that Compound B is used instead of 2,7-diiode-(9,9-dimethyl)-fluorene in Example 125.

### Example 127

### Synthesis of Exemplified Compound No. X-217

Exemplified Compound No. X-217 can be synthesized following the same procedure as in Example 125 with the exception that Compound A is used instead of 2,7-diiode-(9,9-dimethyl)-fluorene in Example 125.

### Example 128

### Synthesis of Exemplified Compound No. X-229

Exemplified Compound No. X-229 can be synthesized following the same procedure as in Example 1 with the exception that: 2,7-diiode-(9,9-dimethyl)-fluorene is used instead of Compound A in Example 1; and Compound Al is used instead of 2-biphenylboric acid in Example 1.

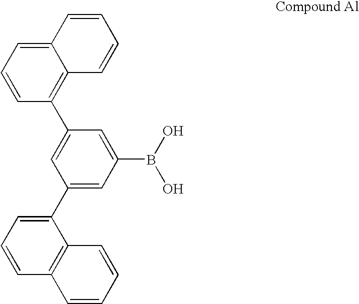

### Example 129

### Synthesis of Exemplified Compound No. X-238

Exemplified Compound No. X-238 can be synthesized following the same procedure as in Example 1 with the exception that Compound B is used instead of Compound A in Example 1 and Compound Am is used instead of 2-biphenylboric acid in Example 1.

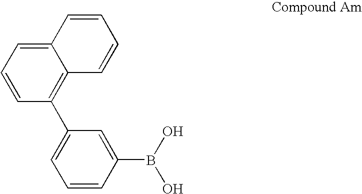

### Example 130

### Synthesis of Exemplified Compound No. X-242

Exemplified Compound No. X-242 can be synthesized following the same procedure as in Example 1 with the exception that Compound B is used instead of Compound A in Example 1 and Compound An is used instead of 2-biphenylboric acid in Example 1.

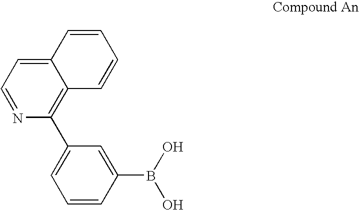

### Example 131

### Synthesis of Exemplified Compound No. X-244

Exemplified Compound No. X-244 can be synthesized following the same procedure as in Example 1 with the exception that Compound B is used instead of Compound A in Example 1 and Compound Ao is used instead of 2-biphenylboric acid in Example 1.

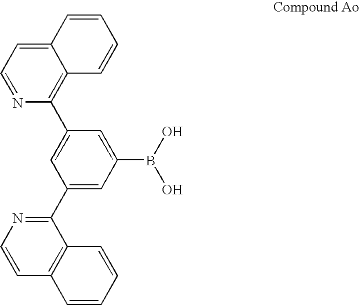

### Example 132

### Synthesis of Exemplified Compound No. X-252

Exemplified Compound No. X-252 can be synthesized following the same procedure as in Example 1 with the exception that Compound Am is used instead of 2-biphenylboric acid in Example 1.

### Example 133

### Synthesis of Exemplified Compound No. X-265

Exemplified Compound No. X-265 can be synthesized following the same procedure as in Example 1 with the exception that Compound Ap is used instead of Compound A in Example 1.

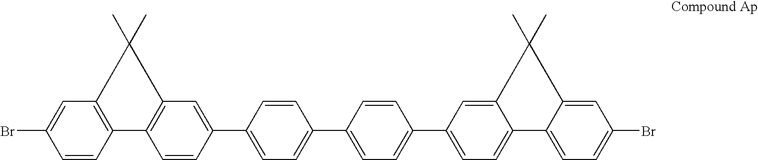

### Example 134

### Synthesis of Exemplified Compound No. X-280

Exemplified Compound No. X-280 can be synthesized following the same procedure as in Example 1 with the exception that Compound Ap is used instead of Compound A in Example 1 and Compound Am is used instead of 2-biphenylboric acid in Example 1.

### Example 135

### Synthesis of Exemplified Compound No. X-363

Exemplified Compound No. X-363 can be synthesized following the same procedure as in Example 1 with the exception that Compound Aq is used instead of Compound A in Example 1 and Compound Am is used instead of 2-biphenylboric acid in Example 1.

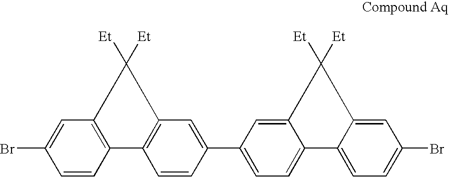

### Example 136

### Synthesis of Exemplified Compound No.

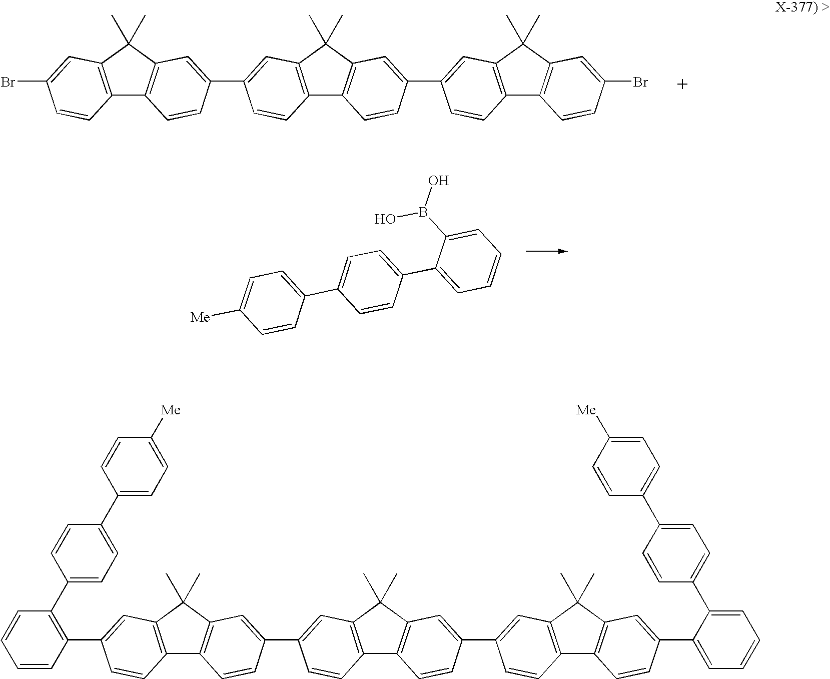 X-377

1 g (1.4 mmole) of Compound A, 938.9 mg (3.25 mmole) of 1,1′:4′,1″,4″-methyl-t-riphenyl-2-boric acid, 350 mg of Pd(PPh₃)₄, 30 ml of toluene, 15 ml of ethanol, and 30 ml of a 2M aqueous solution of sodium carbonate were fed into a 200-ml round-bottomed flask, and the whole was stirred at 80° C. for 8 hours in a stream of nitrogen. After the completion of the reaction, the resultant was extracted with toluene, and the organic layer was dried with magnesium sulfate. After that, the drying agent was filtered and the solvent was distilled off. The residue was dissolved into chloroform, and the solution was separated and purified by means of alumina column chromatography, followed by recrystallization from toluene. The resultant crystal was vacuum-dried at 120° C. to give 980 mg of Exemplified Compound No. X-377 (67% yield).

1062.5 as M+ of the compound was observed by means of Matrix Assisted Laser Desorption/Ionization-Time of Flight Mass Spectrometry (MALDI-TOF MS).

In addition, the structure of the compound was identified by NMR measurement.

¹H NMR (CDCl₃, 400 MHz) σ (ppm): 7.79 (dd, 4H), 7.70 (m, 4H), 7.64-7.35 (m, 28H), 7.22-7.17 (m, 8H), 7.02 (dd, 2H), 2.36 (s, 6H), 1.62 (s, 6H), 1.28 (s, 12H)

### Example 137

### Synthesis of Exemplified Compound No. X-378

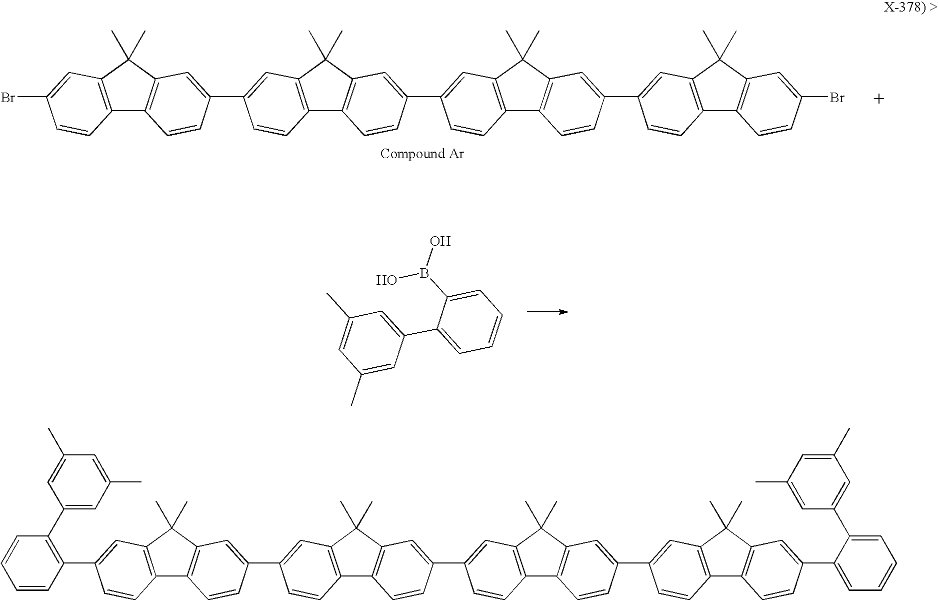

1.5 g (1.6 mmole) of Compound Ar, 800 mg (3.54 mmole) of 3′,5′-dimethylbipheny-2-boric acid, 400 mg of Pd(PPh₃)₄, 30 ml of toluene, 15 ml of ethanol, and 30 ml of a 2M aqueous solution of sodium carbonate were fed into a 200-ml round-bottomed flask, and the whole was stirred at 80° C. for 8 hours in a stream of nitrogen. After the completion of the reaction, the resultant was extracted with toluene, and the organic layer was dried with magnesium sulfate. After that, the drying agent was filtered and the solvent was distilled off. The residue was dissolved into chloroform, and the solution was separated and purified by means of alumina column chromatography, followed by recrystallization from toluene. The resultant crystal was vacuum-dried at 120° C. to give 1.1 g of Exemplified Compound No. X-378 (60% yield).

1131.5 as M+ of the compound was observed by means of Matrix Assisted Laser Desorption/Ionization-Time of Flight Mass Spectrometry (MALDI-TOF MS).

In addition, the structure of the compound was identified by NMR measurement.

¹H NMR (CDCl₃, 400 MHz) σ (ppm): 7.85-7.62 (m, 20H), 7.53 (m, 2H), 7.47-7.40 (m, 6H), 7.28 (dd, 2H), 7.07 (brs, 2H), 6.81 (brs, 2H), 6.89 (brs, 4H), 2.16 (s, 12H), 1.65 (s, 12H), 1.34 (s, 12H)

### Example 138

A device was produced following the same procedure as in Example 2 with the exception that Exemplified Compound No. X-5 was used instead of Exemplified Compound No. X-25; Ir(ppy)₃ (weight ratio: 11%) was used instead of Ir(4mopiq)₃ (weight ratio: 4%) and Ir(bq)₃ (weight ratio: 8%); the thickness of the light-emitting layer was 20 nm; and the thickness of the electron-transporting layer was 30 nm.

The device of this example had an efficiency of 34.6 cd/A, 32.2 lm/W (1200 cd/m²). In addition, the device showed a current value of 24.7 mA/cm² when a voltage of 4 V was applied. When the device was continuously energized at 30 mA/cm², it took 60 hours to reduce an initial luminance of 6500 cd/m² in half.

### Comparative Example 3

A device was produced following the same procedure as in Example 138 with the exception that CBP was used instead of Exemplified Compound No. X-5.

The device of this example had an efficiency of 32.1 cd/A, 28.2 lm/W (1200 cd/m²). In addition, the device showed a current value of 22.2 mA/cm² when a voltage of 4 V was applied. When the device was continuously energized at 30 mA/cm², it took 35 hours to reduce an initial luminance of 6300 cd/m² in half.

Table 3 summarizes the device characteristics of Example 138 and Comparative Example 3.

TABLE 3

| | Light- | Glass | | Current | Half- |
|---|---|---|---|---|---|
| | emitting | transition | Efficiency | value | value |
| | layer | temperature | (lm/W) at | (mA/cm²) | time |
| | host | (° C.) | 1200 cd/m² | at 4 V | (h) |
| Ex. 138 | X-5 | 80 | 32.2 | 24.7 | 60 |
| Comp. Ex. 3 | CBP | 115 | 28.2 | 22.2 | 35 |

As shown in Table 3, the organic EL device using the compound of the present invention for the host of the light-emitting layer is an excellent device which has a power efficiency higher than that of the device using CBP and a half life about twice that of the device using CBP. In addition, the organic EL device shows a higher current value than that of the device using CBP at the same voltage value. Therefore, the organic EL device using the compound of the present invention is extremely excellent in that it shows a larger current value at the same voltage value and can be driven at a lower voltage.

### Example 139

A device was produced following the same procedure as in Example 2 with the exception that Ir(4F5MPiq)₃ (weight ratio: 14%) was used instead of Ir(4mopiq)₃ (weight ratio: 4%) and Ir(bq)₃ (weight ratio: 8%); and the thickness of the light-emitting layer was 25 nm.

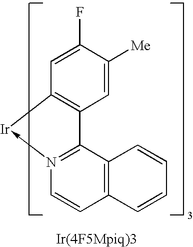

The device of this example had an efficiency of 14.8 cd/A, 13.1 lm/W (600 cd/m²). In addition, the device showed a current value of 14 mA/cm² when a voltage of 4 V was applied. When the device was continuously energized at 100 mA/cm², it took 250 hours to reduce an initial luminance of 7300 cd/m² in half.

### Comparative Example 4

A device was produced following the same procedure as in Example 139 with the exception that CBP was used instead of Exemplified Compound No. X-25.

The device of this example had an efficiency of 8.0 cd/A, 6.0 lm/W (600 cd/m²). In addition, the device showed a current value of 13 mA/cm² when a voltage of 4 V was applied. When the device was continuously energized at 100 mA/cm², it took 50 hours to reduce an initial luminance of 4000 cd/m² in half.

Table 4 summarizes the device characteristics of Example 139 and Comparative Example 4.

TABLE 4

| | Light- | Glass | | Current | Half- |
|---|---|---|---|---|---|
| | emitting | transition | Efficiency | value | value |
| | layer | temperature | (lm/W) at | (mA/cm²) | time |
| | host | (° C.) | 600 cd/m² | at 4 V | (h) |
| Ex. 139 | X-25 | 154 | 13.1 | 14 | 250 |
| Comp. Ex. 4 | CBP | 115 | 6.0 | 13 | 50 |

As shown in Table 4, the organic EL device using the compound of the present invention for the host of the light-emitting layer is an excellent device which has a power efficiency higher than that of the device using CBP and a half life about five times that of the device using CBP.

### Example 140

A device was produced following the same procedure as in Example 2 with the exception that Exemplified Compound No. X-19 was used instead of Exemplified Compound No. X-25; Ir(4F5MPiq)₃ (weight ratio: 14%) was used instead of Ir(4mopiq)₃ (weight ratio: 4%) and Ir(bq)₃ (weight ratio: 8%); and the thickness of the light-emitting layer was 30 nm.

The device of this example had an efficiency of 14.6 cd/A, 11.1 lm/W (600 cd/m²). When the device was continuously energized at 100 mA/cm², it took 100 hours to reduce an initial luminance of 6500 cd/m² in half.

### Example 141

A device was produced following the same procedure as in Example 2 with the exception that Exemplified Compound No. X-20 was used instead of Exemplified Compound No. X-25; Ir(4F5MPiq)₃ (weight ratio: 14%) was used instead of Ir(4mopiq)₃ (weight ratio: 4%) and Ir(bq)₃ (weight ratio: 8%); and the thickness of the light-emitting layer was 35 nm.

The device of this example had an efficiency of 13.0 cd/A, 10.0 lm/W (600 cd/m²). When the device was continuously energized at 100 mA/cm², it took 150 hours to reduce an initial luminance of 6000 cd/m² in half.

### Example 142

A device was produced following the same procedure as in Example 2 with the exception that Exemplified Compound No. X-31 was used instead of Exemplified Compound No. X-25; Ir(4F5MPiq)₃ (weight ratio: 14%) was used instead of Ir(4mopiq)₃ (weight ratio: 4%) and Ir(bq)₃ (weight ratio: 8%); and the thickness of the light-emitting layer was 25 nm.

The device of this example had an efficiency of 12.8 cd/A, 11.0 lm/W (600 cd/m²). When the device was continuously energized at 100 mA/cm², it took 110 hours to reduce an initial luminance of 6500 cd/m² in half.

### Example 143

A device was produced following the same procedure as in Example 2 with the exception that Ir(ppy)₃ (weight ratio: 16%) was used instead of Ir(bq)₃ (weight ratio: 8%).

The device of this example had an efficiency of 17.3 cd/A, 14.0 lm/W (600 cd/m²). When the device was continuously energized at 100 mA/cm², it took 130 hours to reduce an initial luminance of 8100 cd/m² in half.

This application claims priority from Japanese Patent Application Nos. 2004-283238 filed on Sep. 29, 2004 and 2005-234360 filed on Aug. 12, 2005, which are hereby incorporated by reference herein.

1. A compound represented by the general formula (1): 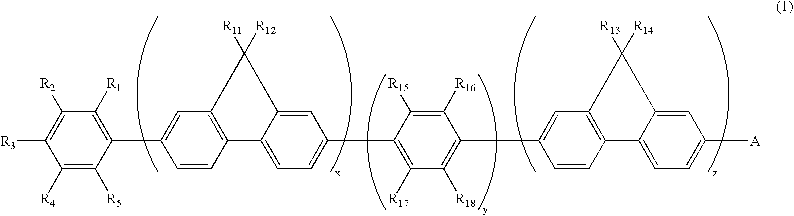
wherein
x, y and z are each independently an integer of 0 to 3 with the proviso that the relation of x+z≧1 is satisfied;
R₃, R₁₅, R₁₆, R₁₇, and R₁₈ are each independently a hydrogen atom or a linear or branched alkyl group, and each CH on the benzene ring having R₁₅, R₁₆, R₁₇, and R₁₈ may independently be replaced by a nitrogen atom;
R₁, R₂, R₄, and R₅ are each independently a hydrogen atom, a linear or branched alkyl group, or a substituted or unsubstituted aryl group with the proviso that at least one of R₁, R₂, R₄, and R₅ is a substituted or unsubstituted aryl group, and each CH on the benzene skeleton constituting the aryl group and each CH on the benzene ring having R₁, R₂, R₃, R₄, and R₅ may independently be replaced by a nitrogen atom;
A is a hydrogen atom, a linear or branched alkyl group, or group B represented by the general formula: 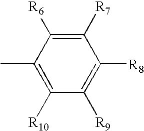
(wherein R₆, R₇, R₈, R₉, and R₁₀ are each independently a hydrogen atom, a linear or branched alkyl group, or a substituted or unsubstituted aryl group, and each CH on the benzene ring having R₆, R₇, R₈, R₉, and R₁₀ and each CH on the benzene skeleton constituting the aryl group may independently be replaced by a nitrogen atom); and
R₁₁, R₁₂, R₁₃, and R₁₄ are each independently a hydrogen atom, a linear or branched alkyl group, or a substituted or unsubstituted aryl group.

2. The compound according to claim 1, wherein A is a hydrogen atom or B.

3. The compound according to claim 2, wherein both y and z are 0.

4. An organic electroluminescent device comprising a pair of electrodes, and at least one layer comprising an organic compound provided between the pair of electrodes, wherein at least one of the at least one layer comprising the organic compound comprises at least one of the compounds represented by the general formula (1) as set forth in claim 1.

5. The organic electroluminescent device according to claim 4, wherein the layer comprising the compound represented by the general formula (1) is a light-emitting layer.

6. The organic electroluminescent device according to claim 5, wherein the light-emitting layer comprises at least two compounds including a host and a guest compounds, and the host compound comprises the compound represented by the general formula (1).

7. The organic electroluminescent device according to claim 6, wherein the guest compound is a phosphorescent material.

8. The organic electroluminescent device according to claim 7, comprising the phosphorescent material in plural kinds.

9. The organic electroluminescent device according to claim 7, wherein the phosphorescent material comprises a metal coordination compound.

10. The organic electroluminescent device according to claim 9, wherein the metal coordination compound comprises an iridium coordination compound.

11. A display apparatus comprising the organic electroluminescent device as set forth in claim 4.
